(19) United States
(12) Patent Application Publication
(10) Pub. No.: US 2019/0008869 A1
(43) Pub. Date: Jan. 10, 2019

(54) Therapeutic Combinations of an Antifolate and a BTK Inhibitor

(71) Applicant: Acerta Pharma B.V., Oss (NL)

(72) Inventors: Allard Kaptein, Zaltbommel (NL); Wayne Rothbaum, Delray Beach, FL (US); Brian Lannutti, Solana Beach, CA (US)

(21) Appl. No.: 16/070,089

(22) PCT Filed: Jan. 13, 2017
(86) PCT No.: PCT/IB2017/050198

§ 371 Date: Jul. 13, 2018

Related U.S. Application Data
(60) Provisional application No. 62/278,374, filed on Jan. 13, 2016.
(60) Provisional application No. 62/371,626, filed on Aug. 5, 2016.

(51) Int. Cl. A61K 31/519; A61K 31/4985; A61K 45/06; A61K 39/395; A61P 19/02

(52) U.S. Cl. CPC A61K 31/519; A61K 31/4985; A61P 19/02; A61K 39/3955; A61K 45/06

(57) ABSTRACT

Therapeutic combinations of an antifolate compound and a Bruton's tyrosine kinase (BTK) inhibitor are described. In some embodiments, the invention provides pharmaceutical compositions comprising combinations of an antifolate compound and a BTK inhibitor, and methods of treating a disease using an antifolate compound and a BTK inhibitor, in particular a cancer or an immune, autoimmune, or inflammatory disease. In some embodiments, the invention provides pharmaceutical compositions comprising combinations of an antifolate compound, a PD-1 or a PD-L1 inhibitor, and a BTK inhibitor, and methods of treating a disease using an antifolate compound, a PD-1 or a PD-L1 inhibitor, and a BTK inhibitor, in particular a cancer or an immune, autoimmune, or inflammatory disease.

### FIELD OF THE INVENTION

Therapeutic combinations of a Bruton's tyrosine kinase (BTK) inhibitor and an antifolate compound, and uses of the therapeutic combinations are disclosed herein. In particular, a combination of a BTK inhibitor and an antifolate compound and compositions and uses thereof are disclosed.

### BACKGROUND OF THE INVENTION

B lymphocyte activation is key in the generation of adaptive immune responses. Derailed B lymphocyte activation is a hallmark of many autoimmune disorders and modulation of this immune response is therefore of therapeutic interest. Recently the success of B cell therapies in autoimmune disorders has been established. Treatment of rheumatoid arthritis (RA) patients with rituximab (anti-CD20 therapy) is an accepted clinical therapy. More recent clinical trials show that treatment with rituximab also ameliorates disease symptoms in relapsing remitting multiple sclerosis (RRMS) and systemic lupus erythematosus (SLE) patients. This success supports the potential for future therapies in autoimmune disorders targeting B cell immunity.

Bruton's Tyrosine Kinase (BTK) is a Tec family non-receptor protein kinase expressed in B cells and myeloid cells. The function of BTK in signaling pathways activated by the engagement of the B cell receptor (BCR) and FCER1 on mast cells is well established. Functional mutations in BTK in humans result in a primary immunodeficiency disease characterized by a defect in B cell development with a block between pro- and pre-B cell stages. The result is an almost complete absence of B lymphocytes, causing a pronounced reduction of serum immunoglobulin of all classes. These findings support a key role for BTK in the regulation of the production of auto-antibodies in autoimmune disorders.

Other diseases with an important role for dysfunctional B cells are B cell malignancies. The reported role for BTK in the regulation of proliferation and apoptosis of B cells indicates the potential for BTK inhibitors in the treatment of B cell lymphomas. BTK inhibitors have thus been developed as potential therapies, as described in D'Cruz and Uckun, OncoTargets and Therapy 2013, 6, 161-176.

Antifolates represent one of the most thoroughly studied classes of antineoplastic agents, with aminopterin initially demonstrating clinical activity approximately 50 years ago. Methotrexate was developed shortly thereafter, and today is a standard component of effective chemotherapeutic regimens for malignancies such as lymphoma, breast cancer, and head and neck cancer. Bonnadonna, et al., J. Am. Med. Assoc. 1995, 273, 542-547; Bonnadonna, et al., N. Engl. J. Med. 1995, 332, 901-906; and Hong, et al., Cancer 1983, 52, 206-210. Antifolates inhibit one or several key folate-requiring enzymes of the thymidine and purine biosynthetic pathways, in particular, thymidylate synthase (TS), dihydrofolate reductase (DHFR), and glycinamide ribonucleotide formyltransferase (GARFT), by competing with reduced folates for binding sites of these enzymes. Shih, et al., Advan. Enzyme Regul. 1998, 38, 135-152 and Shih, et al., Cancer Res 1997, 57, 1116-1123.

The present invention provides the unexpected finding that the combination of an antifolate compound and a BTK inhibitor is synergistically effective in the treatment of any of several types of cancers such as leukemia, lymphoma, and solid tumor cancers, as well as inflammatory, immune, and autoimmune disorders. The present invention also provides the unexpected finding that a combination of an antifolate compound and a BTK inhibitor is synergistically effective in the treatment of any of several types of cancers such as leukemia, lymphoma, and solid tumor cancers, as well as inflammatory, immune, and autoimmune disorders. The present invention further provides the unexpected finding that the combination of an anti-CD20 antibody with a BTK inhibitor and an antifolate compound is synergistically effective in the treatment of any of several types of cancers such as leukemia, lymphoma, and solid tumor cancers, as well as inflammatory, immune, and autoimmune disorders.

### SUMMARY OF THE INVENTION

In an embodiment, the invention provides a method of treating a hyperproliferative disorder, comprising co-administering, to a mammal in need thereof, therapeutically effective amounts of (1) an antifolate compound or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof. In an embodiment, the antifolate compound is administered to the mammal before administration of the BTK inhibitor. In an embodiment, the antifolate compound is administered to the mammal simultaneously with the administration of the BTK inhibitor. In an embodiment, the antifolate compound is administered to the mammal after administration of the BTK inhibitor.

In an embodiment, the invention provides a method of treating a hyperproliferative disorder, comprising co-administering, to a mammal in need thereof, therapeutically effective amounts of (1) an antifolate compound or a pharmaceutically acceptable salt, solvate, hydrate, cocrystal, or prodrug thereof, and (2) a BTK inhibitor or a pharmaceutically acceptable salt, solvate, hydrate, cocrystal, or prodrug thereof, wherein the BTK inhibitor is selected from the group consisting of:

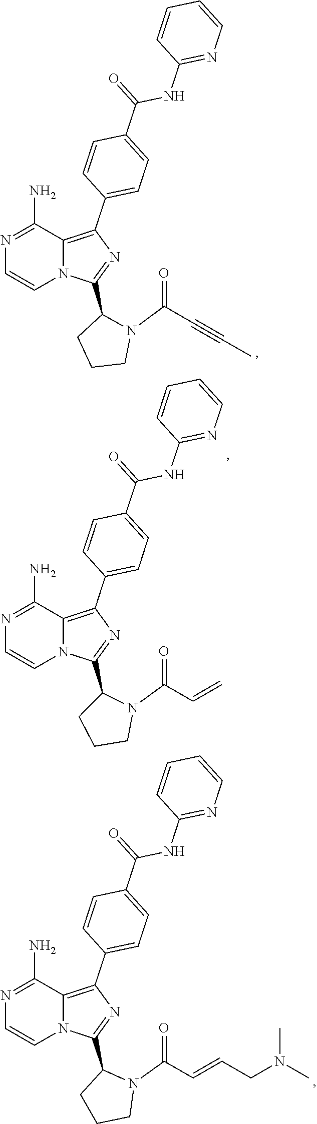

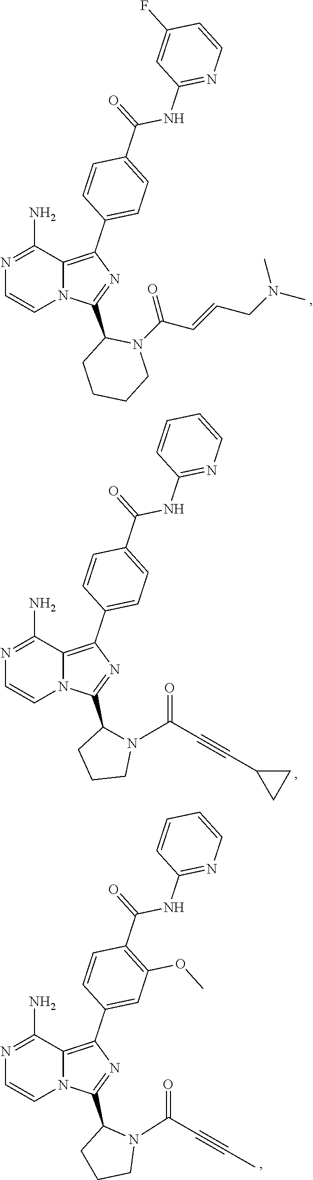

and pharmaceutically-acceptable salts, cocrystals, hydrates, solvates, and prodrugs thereof. In an embodiment, the antifolate compound is administered to the mammal before administration of the BTK inhibitor. In an embodiment, the antifolate compound is administered to the mammal simultaneously with the administration of the BTK inhibitor. In an embodiment, the antifolate compound is administered to the mammal after administration of the BTK inhibitor.

In an embodiment, the invention provides a method of treating a hyperproliferative disorder, comprising co-administering, to a mammal in need thereof, therapeutically effective amounts of (1) an antifolate compound or a pharmaceutically acceptable salt, solvate, hydrate, cocrystal, or prodrug thereof, and (2) a BTK inhibitor or a pharmaceutically acceptable salt, solvate, hydrate, cocrystal, or prodrug thereof, wherein the antifolate compound is selected from the group consisting of methotrexate, pemetrexed, raltitrexed, and pharmaceutically acceptable salts, solvates, hydrates, cocrystals, prodrugs, and combinations thereof.

In an embodiment, the invention provides a method of treating a hyperproliferative disorder, wherein the hyperproliferative disorder is a cancer, comprising co-administering, to a mammal in need thereof, therapeutically effective amounts of (1) an antifolate compound or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof, wherein the cancer is a B cell hematological malignancy, and wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, and myelofibrosis. In an embodiment, the cancer is a solid tumor cancer, wherein the solid tumor cancer is selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer.

In an embodiment, the invention provides a method of treating a hyperproliferative disorder, wherein the hyperproliferative disorder is an inflammatory, immune or autoimmune disorder, comprising co-administering, to a mammal in need thereof, therapeutically effective amounts of (1) an antifolate compound or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof, wherein the inflammatory, immune or autoimmune disorder is selected from the group consisting of tumor angiogenesis, chronic inflammatory disease, rheumatoid arthritis, atherosclerosis, inflammatory bowel disease, skin diseases such as psoriasis, eczema, and sclerodermnna, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polycndocrine autoimmunity, iatrogenic autoimmunity, idiopathic hypoparathyroidism, and vitiligo.

In an embodiment, the invention provides a method of treating a cancer in a human comprising the step of co-administering (1) a therapeutically effective amount of an antifolate compound or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein the therapeutically effective amount is effective to inhibit signaling between the tumor cells of the cancer and at least one tumor microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In an embodiment, the cancer is a solid tumor cancer selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer. In an embodiment, the BTK inhibitor is selected from the group consisting of:

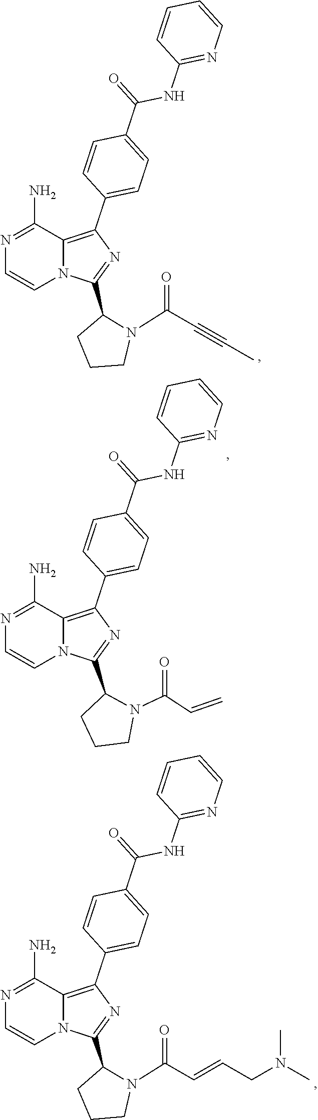

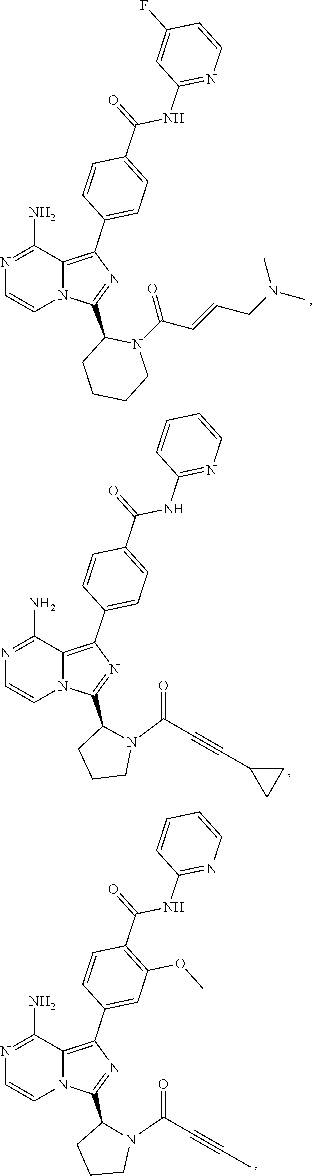

and pharmaceutically-acceptable salts, cocrystals, hydrates, solvates, or prodrugs thereof. In an embodiment, the antifolate compound is selected from the group consisting of methotrexate, pemetrexed, raltitrexed, and pharmaceutically acceptable salts, solvates, hydrates, cocrystals, prodrugs, and combinations thereof.

In an embodiment, the invention provides a method of treating a hyperproliferative disorder in a human intolerant to a bleeding event comprising the step of administering (1) a therapeutically effective amount of an antifolate compound or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein the BTK inhibitor is selected from the group consisting of:

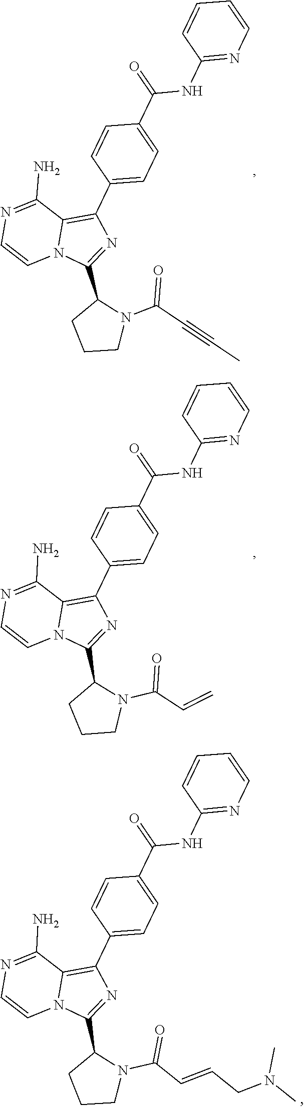

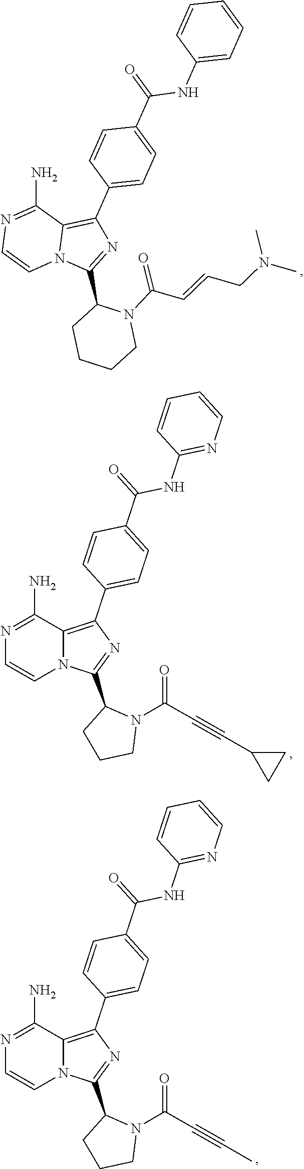 [SMILES: CN(C)C/C=C/C(=O)N1CCCC[C@H]1c1nc(-c2ccc(C(=O)Nc3ccccc3)cc2)c2c(N)nccn12 ; Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)cc2)c2c(N)nccn12]

and pharmaceutically-acceptable salts, cocrystals, hydrates, solvates, or prodrugs thereof. In an embodiment, the bleeding event is selected from the group consisting of subdural hematoma, gastrointestinal bleeding, hematuria, post-procedural hemorrhage, bruising, petechiae, and combinations thereof. In an embodiment, the antifolate compound is selected from the group consisting of methotrexate, pemetrexed, raltitrexed, and pharmaceutically acceptable salts, solvates, hydrates, cocrystals, prodrugs, and combinations thereof. In an embodiment, the hyperproliferative disorder is cancer. In an embodiment, the hyperproliferative disorder is an inflammatory, immune or autoimmune disorder.

In an embodiment, the invention provides a method of treating a hyperproliferative disorder in a human intolerant to a bleeding event comprising the step of administering (1) a therapeutically effective amount of an antifolate compound or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, further comprising the step of administering a therapeutically effective amount of an anticoagulant or antiplatelet active pharmaceutical ingredient. In an embodiment, the anticoagulant or antiplatelet active pharmaceutical ingredient is selected from the group consisting of acenocoumarol, anagrelide, anagrelide hydrochloride, abciximab, aloxiprin, antithrombin, apixaban, argatroban, aspirin, aspirin with extended-release dipyridamole, beraprost, betrixaban, bivalirudin, carbasalate calcium, cilostazol, clopidogrel, clopidogrel bisulfate, cloricromen, dabigatran etexilate, darexaban, dalteparin, dalteparin sodium, defibrotide, dicumarol, diphenadione, dipyridamole, ditazole, desirudin, edoxaban, enoxaparin, enoxaparin sodium, eptifibatide, fondaparinux, fondaparinux sodium, heparin, heparin sodium, heparin calcium, idraparinux, idraparinux sodium, iloprost, indobufen, lepirudin, low molecular weight heparin, melagatran, nadroparin, otamixaban, parnaparin, phenindione, phenprocoumon, prasugrel, picotamide, prostacyclin, ramatroban, reviparin, rivaroxaban, sulodexide, terutroban, terutroban sodium, ticagrelor, ticlopidine, ticlopidine hydrochloride, tinzaparin, tinzaparin sodium, tirofiban, tirofiban hydrochloride, treprostinil, treprostinil sodium, triflusal, vorapaxar, warfarin, warfarin sodium, ximelagatran, salts thereof, solvates thereof, hydrates thereof, and combinations thereof. In an embodiment, the hyperproliferative disorder is cancer. In an embodiment, the hyperproliferative disorder is an inflammatory, immune or autoimmune disorder. In an embodiment, the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myetlocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, Burkitt's lymphoma, and myelofibrosis.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor or a pharmaceutically acceptable salt, solvate, hydrate, cocrystal, or prodrug thereof. In an embodiment, the antifolate compound is selected from the group consisting of methotrexate, pemetrexed, raltitrexed, and pharmaceutically acceptable salts, solvates, hydrates, cocrystals, prodrugs, and combinations thereof. This composition is typically a pharmaceutical composition. In some embodiments, the composition is used in the treatment of hyperproliferative disorders. In some embodiments, the composition is used in the treatment of cancer. In other embodiments, the composition is used in the treatment of an inflammatory, immune or autoimmune disorder. In some embodiments, the composition is used to treat arthritis.

In some embodiments, the invention provides a composition comprising (1) a therapeutically effective amount of an antifolate compound or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein, the composition is used in the treatment of a hyperproliferative disorder in a human intolerant to a bleeding event. In some embodiments, the BTK inhibitor is selected from the group consisting of:

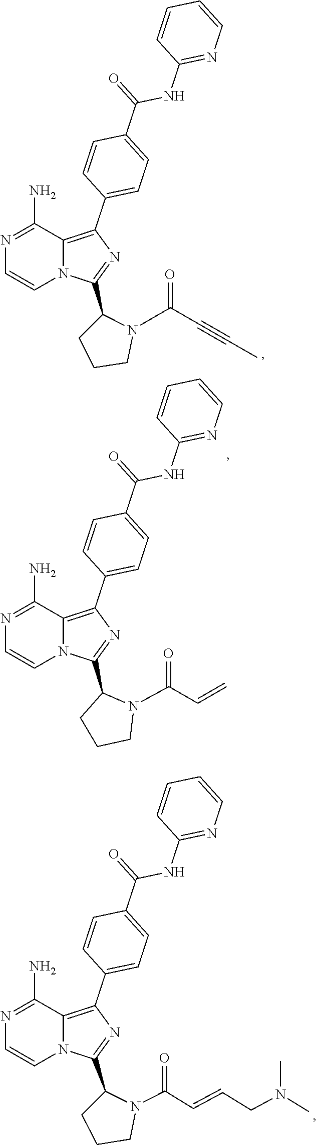

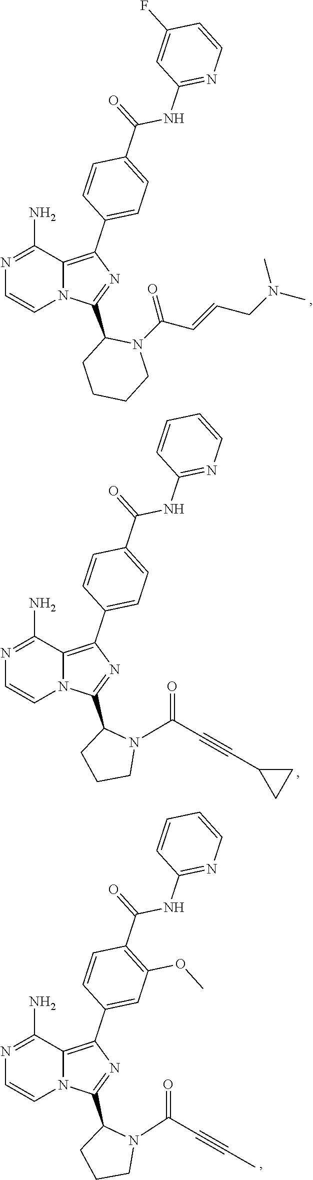

and pharmaceutically-acceptable salts, cocrystals, hydrates, solvates, and prodrugs thereof. In an embodiment, the antifolate compound is selected from the group consisting of methotrexate, pemetrexed, raltitrexed, and pharmaceutically acceptable salts, solvates, hydrates, cocrystals, prodrugs, and combinations thereof.

In some embodiments, the invention provides a composition comprising therapeutically effective amounts of (1) an antifolate compound or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof, for use in the treatment of hyperproliferative disorders; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a method of treating leukemia, lymphoma or a solid tumor cancer in a subject, comprising co-administering to a mammal in need thereof any of the foregoing compositions.

In some embodiments, the invention provides a method of treating leukemia, lymphoma or a solid tumor cancer in a subject, comprising co-administering to a mammal in need thereof a therapeutically effective amount of an antifolate compound and a BTK inhibitor.

In some embodiments, the invention provides a method of treating leukemia, lymphoma or a solid tumor cancer in a subject, comprising co-administering to a mammal in need thereof a therapeutically effective amount of an antifolate compound, a BTK inhibitor, and an anti-CD20 antibody.

In some embodiments, the invention provides a method of treating leukemia, lymphoma or a solid tumor cancer in a subject, comprising co-administering to a mammal in need thereof a therapeutically effective amount of an antifolate compound, a BTK inhibitor, and bendustamine.

In some embodiments, the invention provides a method of treating leukemia, lymphoma or a solid tumor cancer in a subject, comprising co-administering to a mammal in need thereof a therapeutically effective amount of an antifolate compound, a BTK inhibitor, and a combination of cyclophosphamide, doxorubicin, vincristine, and prednisone (CHOP).

In some embodiments, the invention provides a method of treating leukemia, lymphoma or a solid tumor cancer in a subject, comprising co-administering to a mammal in need thereof a therapeutically effective amount of an antifolate compound, a BTK inhibitor, and a combination of rituximab, cyclophosphamide, doxorubicin, vincristine, and prednisone (R-CHOP).

In some embodiments, the invention provides a method of treating leukemia, lymphoma or a solid tumor cancer in a subject, comprising co-administering to a mammal in need thereof a therapeutically effective amount of an antifolate compound, a BTK inhibitor, and a combination of fludarabine, cyclophosphamide, and rituximab (FCR).

### BRIEF DESCRIPTION OF THE DRAWINGS

The foregoing summary, as well as the following detailed description of the invention, will be better understood when read in conjunction with the appended drawings.

FIG. 1 illustrates the effect of the BTK inhibitor of Formula (2) (1 mg/kg), alone or in combination with methotrexate (0.3 mg/kg) on arthritis score versus day following immunization.

FIG. 2 illustrates the effect of the BTK inhibitor of Formula (2) (1 mg/kg), alone or in combination with methotrcxate (0.5 mg/kg) on arthritis score versus day following immunization.

FIG. 3 illustrates the effect of the BTK inhibitor of Formula (2) (5 mg/kg), alone or in combination with methotrexate (0.3 mg/kg) on arthritis score versus day following immunization.

FIG. 4 illustrates the effect of the BTK inhibitor of Formula (2) (5 mg/kg), alone or in combination with methotrexate (0.5 mg/kg) on arthritis score versus day following immunization.

### BRIEF DESCRIPTION OF THE SEQUENCE LISTING

SEQ ID NO:1 is the heavy chain amino acid sequence of the anti-CD20 monoclonal antibody rituximab.

SEQ ID NO:2 is the light chain amino acid sequence of the anti-CD20 monoclonal antibody rituximab.

SEQ ID NO:3 is the heavy chain amino acid sequence of the anti-CD20 monoclonal antibody obinutuzumab.

SEQ ID NO:4 is the light chain amino acid sequence of the anti-CD20 monoclonal antibody obinutuzumab.

SEQ ID NO:5 is the variable heavy chain amino acid sequence of the anti-CD20 monoclonal antibody ofatumumab.

SEQ ID NO:6 is the variable light chain amino acid sequence of the anti-CD20 monoclonal antibody ofatumumab.

SEQ ID NO:7 is the Fab fragment heavy chain amino acid sequence of the anti-CD20 monoclonal antibody ofatumumab.

SEQ ID NO:8 is the Fab fragment light chain amino acid sequence of the anti-CD20 monoclonal antibody ofatumumab.

SEQ ID NO:9 is the heavy chain amino acid sequence of the anti-CD20 monoclonal antibody veltuzumab.

SEQ ID NO:10 is the light chain amino acid sequence of the anti-CD20 monoclonal antibody veltuzumab.

SEQ ID NO:11 is the heavy chain amino acid sequence of the anti-CD20 monoclonal antibody tositumomab.

SEQ ID NO:12 is the light chain amino acid sequence of the anti-CD20 monoclonal antibody tositumomab.

SEQ ID NO:13 is the heavy chain amino acid sequence of the anti-CD20 monoclonal antibody ibritumomab.

SEQ ID NO:14 is the light chain amino acid sequence of the anti-CD20 monoclonal antibody ibritumomab.

SEQ ID NO:15 is the heavy chain amino acid sequence of the PD-1 inhibitor nivolumab.

SEQ ID NO:16 is the light chain amino acid sequence of the PD-1 inhibitor nivolumab.

SEQ ID NO: 17 is the heavy chain variable region (V_{H}) amino acid sequence of the PD-1 inhibitor nivolumab.

SEQ ID NO: 18 is the light chain variable region (V_{L}) amino acid sequence of the PD-1 inhibitor nivolumab.

SEQ ID NO: 19 is the heavy chain CDR1 amino acid sequence of the PD-1 inhibitor nivolumab.

SEQ ID NO:20 is the heavy chain CDR2 amino acid sequence of the PD-1 inhibitor nivolumab.

SEQ ID NO:21 is the heavy chain CDR3 amino acid sequence of the PD-1 inhibitor nivolumab.

SEQ ID NO:22 is the light chain CDR1 amino acid sequence of the PD-1 inhibitor nivolumab.

SEQ ID NO:23 is the light chain CDR2 amino acid sequence of the PD-1 inhibitor nivolumab.

SEQ ID NO:24 is the light chain CDR3 amino acid sequence of the PD-1 inhibitor nivolumab.

SEQ ID NO:25 is the heavy chain amino acid sequence of the PD-1 inhibitor pembrolizumab.

SEQ ID NO:26 is the light chain amino acid sequence of the PD-1 inhibitor pembrolizumab.

SEQ ID NO:27 is the heavy chain variable region (V_{H}) amino acid sequence of the PD-1 inhibitor pembrolizumab.

SEQ ID NO:28 is the light chain variable region (V_{L}) amino acid sequence of the PD-1 inhibitor pembrolizumab.

SEQ ID NO:29 is the heavy chain CDR1 amino acid sequence of the PD-1 inhibitor pembrolizumab.

SEQ ID NO:30 is the heavy chain CDR2 amino acid sequence of the PD-1 inhibitor pembrolizumab.

SEQ ID NO:31 is the heavy chain CDR3 amino acid sequence of the PD-1 inhibitor pembrolizumab.

SEQ ID NO:32 is the light chain CDR1 amino acid sequence of the PD-1 inhibitor pembrolizumab.

SEQ ID NO:33 is the light chain CDR2 amino acid sequence of the PD-1 inhibitor pembrolizumab.

SEQ ID NO:34 is the light chain CDR3 amino acid sequence of the PD-1 inhibitor pembrolizumab.

SEQ ID NO:35 is the heavy chain amino acid sequence of the PD-1 inhibitor pidilizumab.

SEQ ID NO:36 is the light chain amino acid sequence of the PD-1 inhibitor pidilizumab.

SEQ ID NO:37 is the heavy chain variable region (V_{H}) amino acid sequence of the PD-1 inhibitor pidilizumab.

SEQ ID NO:38 is the light chain variable region (V_{L}) amino acid sequence of the PD-1 inhibitor pidilizumab.

SEQ ID NO:39 is the heavy chain amino acid sequence of the PD-L1 inhibitor durvalumab.

SEQ ID NO:40 is the light chain amino acid sequence of the PD-L1 inhibitor durvalumab.

SEQ ID NO:41 is the heavy chain variable region (V_{H}) amino acid sequence of the PD-L1 inhibitor durvalumab.

SEQ ID NO:42 is the light chain variable region (V_{L}) amino acid sequence of the PD-L1 inhibitor durvalumab.

SEQ ID NO:43 is the heavy chain CDR1 amino acid sequence of the PD-L1 inhibitor durvalumab.

SEQ ID NO:44 is the heavy chain CDR2 amino acid sequence of the PD-L1 inhibitor durvalumab.

SEQ ID NO:45 is the heavy chain CDR3 amino acid sequence of the PD-L1 inhibitor durvalumab.

SEQ ID NO:46 is the light chain CDR1 amino acid sequence of the PD-L1 inhibitor durvalumab.

SEQ ID NO:47 is the light chain CDR2 amino acid sequence of the PD-L1 inhibitor durvalumab.

SEQ ID NO:48 is the light chain CDR3 amino acid sequence of the PD-L1 inhibitor durvalumab.

SEQ ID NO:49 is the heavy chain amino acid sequence of the PD-L1 inhibitor atezolizumab.

SEQ ID NO:50 is the light chain amino acid sequence of the PD-L1 inhibitor atezolizumab.

SEQ ID NO:51 is the heavy chain variable region (V) amino acid sequence of the PD-L1 inhibitor atezolizumab.

SEQ ID NO:52 is the light chain variable region (V_{L}) amino acid sequence of the PD-L1 inhibitor atezolizumab.

SEQ ID NO:53 is the heavy chain CDR1 amino acid sequence of the PD-L1 inhibitor atezolizumab.

SEQ ID NO:54 is the heavy chain CDR2 amino acid sequence of the PD-L1 inhibitor atezolizumab.

SEQ ID NO:55 is the heavy chain CDR3 amino acid sequence of the PD-L1 inhibitor atezolizumab.

SEQ ID NO:56 is the light chain CDR1 amino acid sequence of the PD-L1 inhibitor atezolizumab.

SEQ ID NO:57 is the light chain CDR2 amino acid sequence of the PD-L1 inhibitor atezolizumab.

SEQ ID NO:58 is the light chain CDR3 amino acid sequence of the PD-L1 inhibitor atezolizumab.

SEQ ID NO:59 is the heavy chain amino acid sequence of the PD-L1 inhibitor avelumab.

SEQ ID NO:60 is the light chain amino acid sequence of the PD-L1 inhibitor avelumab.

SEQ ID NO:61 is the heavy chain variable region (V_{H}) amino acid sequence of the PD-L1 inhibitor avelumab.

SEQ ID NO:62 is the heavy chain variable region (V_{H}) amino acid sequence of the PD-L1 inhibitor avelumab.

SEQ ID NO:63 is the heavy chain CDR1 amino acid sequence of the PD-L1 inhibitor avelumab.

SEQ ID NO:64 is the heavy chain CDR2 amino acid sequence of the PD-L1 inhibitor avelumab.

SEQ ID NO:65 is the heavy chain CDR3 amino acid sequence of the PD-L1 inhibitor avelumab.

SEQ ID NO:66 is the light chain CDR1 amino acid sequence of the PD-L1 inhibitor avelumab.

SEQ ID NO:67 is the light chain CDR2 amino acid sequence of the PD-L1 inhibitor avelumab.

SEQ ID NO:68 is the light chain CDR3 amino acid sequence of the PD-L1 inhibitor avelumab.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, at least one antifolate compound and at least one BTK inhibitor) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily. The terms “PO”, “po” or “p.o.” mean per os, by mouth or orally.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolaminc. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

As used herein, the term “warhead” or “warhead group” refers to a functional group present on a compound of the present invention wherein that functional group is capable of covalently binding to an amino acid residue present in the binding pocket of the target protein (such as cysteine, lysine, histidine, or other residues capable of being covalently modified), thereby irreversibly inhibiting the protein.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)— R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)— (optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

Compounds of the invention also include antibodies. The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (C1q) of the classical complement system.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to, e.g., CD20, PD-1, PD-L1, or PD-L2 can be made using knowledge and skill in the art of injecting test subjects with CD20, PD-1, PD-L1, or PD-L2 antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion”), as used herein, refers to one or more fragments of an antibody that retain the ability to specifically bind to an antigen (e.g., PD-1, PD-L1, or PD-L2). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to a therapeutic moiety, such as a bacterial toxin, a cytotoxic drug or a radionuclide-containing toxin. Toxic moieties can be conjugated to antibodies of the invention using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosamninyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384.

The term “conservative amino acid substitutions” in means amino acid sequence modifications which do not abrogate the binding of the antibody to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in a PD-1 or PD-L1 antibody is preferably replaced with another amino acid residue from the same class. Methods of identifying amino acid conservative substitutions which do not eliminate antigen binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417.

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

Certain embodiments of the present invention comprise a variant of an antibody, e.g., an antifolate compound that is an antibody, an anti-CD20 antibody, an anti-PD-1 antibody, an anti-PD-L1 antibody, and/or an anti-PD-L2 antibody. As used herein, the term “variant” encompasses but is not limited to antibodies which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody.

The term “radioisotope-labeled complex” refers to both non-covalent and covalent attachment of a radioactive isotope, such as ⁹⁰Y, ¹¹¹In, or ¹³¹I, to an antibody, including conjugates.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference anti-CD20 monoclonal antibody is rituximab, an anti-CD20 biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorised, approved for authorisation or subject of an application for authorisation under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorised by the EMA. However, in some instances, the biosimilar may be compared to a biological medicinal product which has been authorised outside the European Economic Area (a non-EEA authorised “comparator”) in certain studies. Such studies include for example certain clinical and in vivo non-clinical studies. As used herein, the term “biosimilar” also relates to a biological medicinal product which has been or may be compared to a non-EEA authorised comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorised or considered suitable for authorisation. In certain circumstances, the biosimilar exhibits different binding characteristics as compared to the reference medicinal product, wherein the different binding characteristics are considered by a Regulatory Authority such as the EMA not to be a barrier for authorisation as a similar biological product. The term “biosimilar” is also used synonymously by other national and regional regulatory agencies.

The term “antifolate compound” as used herein includes a compound inhibits one or more folate-requiring enzymes of the thymidine and purine biosynthetic pathways, including thymidylate synthase (TS), dihydrofolate reductase (DHFR), and glycinamide ribonucleotide formyltransferase.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, ALL, CLL, SLL, acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

The term “microenvironment,” as used herein, may refer to the tumor microenvironment as a whole or to an individual subset of cells within the microenvironment.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

### Co-Administration of Compounds

In an embodiment, the invention includes a composition, such as a pharmaceutical composition, comprising a combination of a BTK inhibitor and an antifolate compound. In some embodiments, the composition further includes an anti-CD20 antibody. In some other embodiments, the composition further includes a chemotherapeutic agent. In some other embodiments, the composition further includes a PD-1 or PD-L1 inhibitor.

Another aspect of the invention is a kit containing a BTK inhibitor and an antifolate compound, wherein each of the inhibitor and antifolate compound is formulated into a separate pharmaceutical composition, and wherein said separate pharmaceutical compositions are formulated for co-administration. Preferably, said kit contains a BTK inhibitor and an antifolate compound.

Another aspect of the invention is a method of treating a disease or condition in a subject, in particular a hyperproliferative disorder such as leukemia, lymphoma or a solid tumor cancer in a subject or an inflammatory, immune or autoimmune disorder in a subject, comprising co-administering to the subject in need thereof a therapeutically effective amount of a combination of a BTK inhibitor and an antifolate compound. In some embodiments, the combination further includes an anti-CD20 antibody. In some other embodiments, the combination further includes a chemotherapeutic agent. In some other embodiments, the combination further includes a PD-1 or PD-L1 inhibitor. In an embodiment, the foregoing method exhibits synergistic effects that may result in greater efficacy, less side effects, the use of less active pharmaceutical ingredient to achieve a given clinical result, or other synergistic effects. A combination of a BTK inhibitor and an antifolate compound is a preferred embodiment. The pharmaceutical composition comprising the combination, and the kit, are both for use in treating such disease or condition.

In a preferred embodiment, the solid tumor cancer is selected from the group consisting of breast, lung, colorectal, thyroid, bone sarcoma, pancreatic, and stomach cancers.

In a preferred embodiment, the leukemia is selected from the group consisting of acute myelogenous leukemia (AML), chronic myelogenous leukemia (CML), acute lymphoblastic leukemia (ALL), B cell chronic lymphocytic leukemia (B-CLL), and chronic lymphoid leukemia (CLL).

In a preferred embodiment, the lymphoma is selected from the group consisting of Burkitt's lymphoma, mantle cell lymphoma, follicular lymphoma, indolent B-cell non-Hodgkin's lymphoma, histiocytic lymphoma, activated B-cell like diffuse large B cell lymphoma (DLBCL-ABC), germinal center B-cell like diffuse large B cell lymphoma (DLBCL-GCB), and diffuse large B cell lymphoma (DLBCL).

In a preferred embodiment, the inflammatory, immune or autoimmune disorder is arthritis.

In an embodiment, the combination of the BTK inhibitor and the antifolate compound is administered by oral, intravenous, intramuscular, intraperitoneal, subcutaneous, or transdermal means.

In an embodiment, the BTK inhibitor is in the form of a pharmaceutically acceptable salt, solvate, hydrate, complex, derivative, prodrug (such as an ester or phosphate ester), or cocrystal.

In an embodiment, the antifolate compound is in the form of a pharmaceutically acceptable salt, solvate, hydrate, complex, derivative, prodrug.

In an embodiment, the antifolate compound is administered to the subject before administration of the BTK inhibitor.

In an embodiment, the antifolate compound is administered concurrently with the administration of the BTK inhibitor.

In an embodiment, the antifolate compound is administered to the subject after administration of the BTK inhibitor.

In a preferred embodiment, the subject is a mammal, such as a human. In an embodiment, the subject is a human. In an embodiment, the subject is a companion animal. In an embodiment, the subject is a canine, feline, or equine.

### BTK Inhibitors

The BTK inhibitor may be any BTK inhibitor known in the art. In particular, it is one of the BTK inhibitors described in more detail in the following paragraphs.

In an embodiment, the BTK inhibitor is a compound of Formula (1):

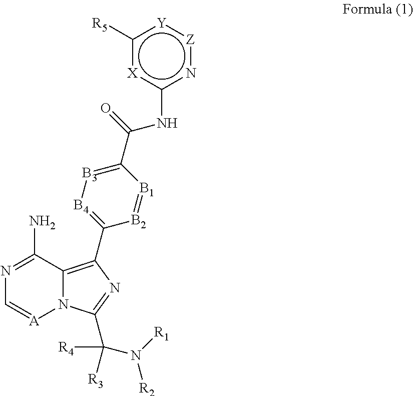

or a pharmaceutically acceptable salt, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
B₂ is N or C(R⁸);
B₃ is N or C(R₉);
B₄ is N or C(R₁₀);
R₁ is R₁₁C(═O), R₁₂S(═O), R₁₃S(═O)₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₄)alkyl, (C₁₋₃)alkoxy, (C₃₋₆)cycloalkyl, any alkyl group of which is optionally substituted with one or more halogen; or R₅ is (C₆₋₁₀)aryl or (C₂₋₆)heterocycloalkyl;
R₆ is H or (C₁₋₃)alkyl; or
R₅ and R₆ together may form a (C₃₋₇)cycloalkenyl or (C₂₋₆)heterocycloalkenyl, each optionally substituted with (C₁₋₃)alkyl or one or more halogens;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₉)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy;
R₁₁ is independently selected from the group consisting of (C₁₋₆)alkyl, (C₂₋₆)alkenyl and (C₂₋₆)alkynyl, where each alkyl, alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or R₁₁ is (C₁₋₃)alkyl-C(O)—S—(C₁₋₃)alkyl; or
R₁₁ is (C₁₋₅)heteroaryl optionally substituted with one or more substituents selected from the group consisting of halogen or cyano;
R₁₂ and R₁₃ are independently selected from the group consisting of (C₂₋₆)alkenyl or (C₂₋₆)alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or a (C₁₋₅)heteroaryl optionally substituted with one or more substituents selected from the group consisting of halogen and cyano; and
R₁₄ is independently selected from the group consisting of halogen, cyano, (C₂₋₆)alkenyl and (C₂₋₆)alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, (C₁₋₅)heteroaryl and (C₃₋₇)heterocycloalkyl;
   with the proviso that:
0 to 2 atoms of X, Y, Z can simultaneously be a heteroatom;
when one atom selected from X, Y is O or S, then Z is a bond and the other atom selected from X, Y cannot be O or S;
when Z is C or N then Y is C(R₆) or N and X is C or N;
0 to 2 atoms of B₁, B₂, B₃ and B₄ are N;
with the terms used having the following meanings:
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined. (C₁₋₂)alkoxy groups are preferred;
(C₂₋₆)alkenyl means a branched or unbranched alkenyl group having 2-6 carbon atoms, such as ethenyl, 2-butenyl, and n-pentenyl, (C₂₋₄)alkenyl groups being most preferred;
(C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl. (C₂₋₄)alkynyl groups are preferred; (C₃₋₆)cycloalkyl means a cycloalkyl group having 3-6 carbon atoms, being cyclopropyl, cyclobutyl, cyclopentyl or cyclohexyl;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; also preferred are piperidine, morpholine, pyrrolidine and piperazine; with the most preferred (C₂₋₆)heterocycloalkyl being pyrrolidine; the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S. Preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S; the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, a more preferred (C₁₋₅)heteroaryl is pyrimidyl;
[(C₁₋₄)alkyl]amino means an amino group, monosubstituted with an alkyl group containing 1-4 carbon atoms having the same meaning as previously defined; preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; preferred di[(C₁₋₄)alkyl]amino group is dimethylamino;
halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; preferred (C₂₋₆)heterocycloalkenyl groups are oxycyclohexenyl and azacyclohexenyl group.
In the above definitions with multifunctional groups, the attachment point is at the last group.
When, in the definition of a substituent, it is indicated that “all of the alkyl groups” of said substituent are optionally substituted, this also includes the alkyl moiety of an alkoxy group.
A circle in a ring of Formula (1) indicates that the ring is aromatic.
Depending on the ring formed, the nitrogen, if present in X or Y, may carry a hydrogen.

In a preferred embodiment, the BTK inhibitor is a compound of Formula (1) or a pharmaceutically acceptable salt thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is R₁₁C(═O),
R₂ is (C₁₋₃)alkyl;
R₃ is (C₁₋₃)alkyl; or
R₂ and R₃ form, together with the N and C atom they are attached to, a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R⁴ is H;
R₅ is H, halogen, cyano, (C₁₋₄)alkyl, (C₁₋₃)alkoxy, (C₃₋₆)cycloalkyl, or an alkyl group which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
R₁₁ is independently selected from the group consisting of (C₂₋₆)alkenyl and (C₂₋₆)alkynyl, where each alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl;
with the proviso that 0 to 2 atoms of B₁, B₂, B₃ and B₄ are N.

In an embodiment of Formula (1), B₁ is C(R₇); B₂ is C(R₈); B₃ is C(R₉); B₄ is C(R₁₀); R₇, R₉, and R₁₀ are each H; and R₈ is hydrogen or methyl.

In an embodiment of Formula (1), the ring containing X, Y and Z is selected from the group consisting of pyridyl, pyrimidyl, pyridazyl, triazinyl, thiazolyl, oxazolyl and isoxazolyl.

In an embodiment of Formula (1), the ring containing X, Y and Z is selected from the group consisting of pyridyl, pyrimidyl and pyridazyl.

In an embodiment of Formula (1), the ring containing X, Y and Z is selected from the group consisting of pyridyl and pyrimidyl.

In an embodiment of Formula (1), the ring containing X, Y and Z is pyridyl.

In an embodiment of Formula (1), R₅ is selected from the group consisting of hydrogen, fluorine, methyl, methoxy and trifluoromethyl.

In an embodiment of Formula (1), R₅ is hydrogen.

In an embodiment of Formula (1), R₂ and R₃ together form a heterocycloalkyl ring selected from the group consisting of azetidinyl, pyrrolidinyl, piperidinyl, homopiperidinyl and morpholinyl, optionally substituted with one or more of fluoro, hydroxyl, (C₁₋₃)alkyl and (C₁₋₃)alkoxy.

In an embodiment of Formula (1), R₂ and R₃ together form a heterocycloalkyl ring selected from the group consisting of azetidinyl, pyrrolidinyl and piperidinyl.

In an embodiment of Formula (1), R₂ and R₃ together form a pyrrolidinyl ring.

In an embodiment of Formula (1), R₁ is independently selected from the group consisting of (C₁₋₆)alkyl, (C₂₋₆)alkenyl or (C₂₋₆)alkynyl, each optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl] amino, (C₁₋₃)alkoxy, (C₃₋₇)cycloalkoxy, (C₆₋₁₀)aryl and (C₃₋₇)heterocycloalkyl.

In an embodiment of Formula (1), B₁, B₂, B₃ and B₄ are CH; X is N; Y and Z are CH; R₅ is CH₃; A is N; R₂, R₃ and R₄ are H; and R₁ is CO—CH₃.

In an embodiment of Formula (1), B₁, B₂, B₃ and B₄ are CH; X and Y are N; Z is CH; R₅ is CH₃; A is N; R₂, R₃ and R₄ are H; and R₁ is CO—CH₃.

In an embodiment of Formula (1), B₁, B₂, B₃ and B₄ are CH; X and Y are N; Z is CH; R₅ is CH₃; A is CH; R₂ and R₃ together form a piperidinyl ring; R₄ is H; and R₁ is CO— ethenyl.

In an embodiment of Formula (1), B₁, B₂, B₃ and B₄ are CH; X, Y and Z are CH; R₅ is H; A is CH; R₂ and R₃ together form a pyrrolidinyl ring; R₄ is H; and R₁ is CO-propynyl.

In an embodiment of Formula (1), B₁, B₂, B₃ and B₄ are CH; X, Y and Z are CH; R₅ is CH₃; A is CH; R₂ and R₃ together form a piperidinyl ring; R₄ is H; and R₁ is CO-propynyl.

In an embodiment of Formula (1), B₁, B₂, B₃ and B₄ are CH; X and Y are N; Z is CH; R₅ is H; A is CH; R₂ and R₃ together form a morpholinyl ring; R₄ is H; and R₁ is CO-ethenyl.

In an embodiment of Formula (1), B₁, B₂, B₃ and B₄ are CH; X and Y are N; Z is CH; R₅ is CH₃; A is CH; R₂ and R₃ together form a morpholinyl ring; R₄ is H; and R₁ is CO-propynyl.

In a preferred embodiment, the BTK inhibitor is a compound of Formula (2):

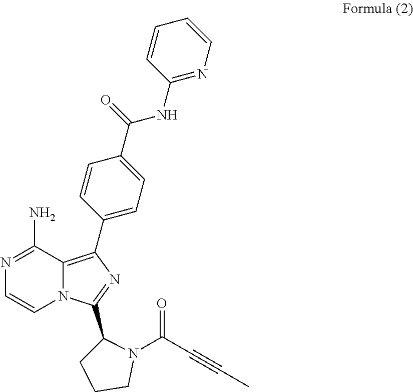 [SMILES: CC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is (S)-4-(8-amino-3-(1-(but-2-ynoyl)pyrrolidin-2-yl)imidazo[1,5-a]pyrazin-1-yl)-N-(pyridin-2-yl)benzamide or pharmaceutically acceptable salt, solvate, hydrate, cocrystal, or prodrug thereof.

In a preferred embodiment, the BTK inhibitor is a compound of Formula (3):

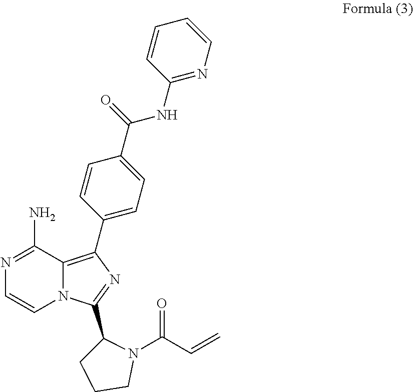 [SMILES: C=CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (4):

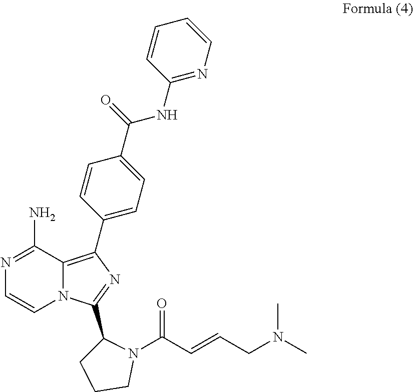 [SMILES: CN(C)C/C=C/C(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (5):

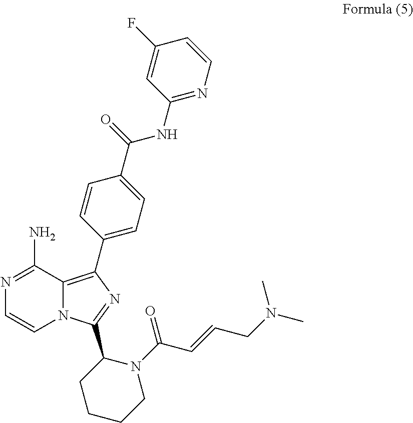 [SMILES: CN(C)C/C=C/C(=O)N1CCCC[C@H]1c1nc(-c2ccc(C(=O)Nc3cc(F)ccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (6):

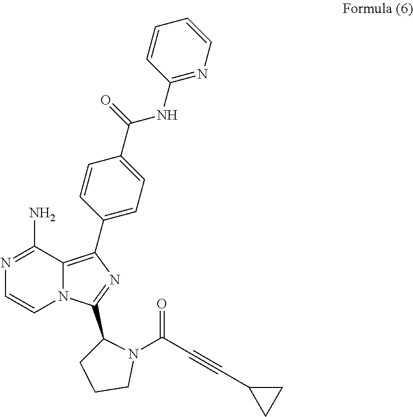 [SMILES: Nc1nccn2c([C@@H]3CCCN3C(=O)C#CC3CC3)nc(-c3ccc(C(=O)Nc4ccccn4)cc3)c12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (7):

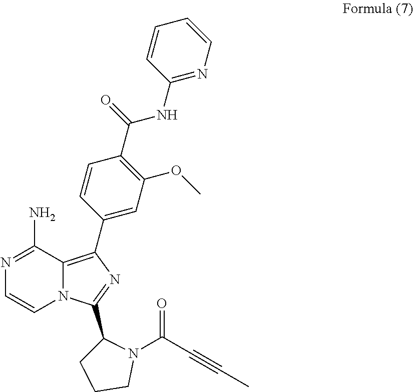 [SMILES: CC#CC(=O)N1CCC[C@H]1c1nc(-c2ccc(C(=O)Nc3ccccn3)c(OC)c2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In other embodiments, the BTK inhibitors include, but are not limited to, those compounds described in International Patent Application Publication No. WO 2013/010868 and U.S. Patent Application Publication No. US 2014/0155385 A1, the disclosures of each of which are specifically incorporated by reference herein.

In an embodiment, the BTK inhibitor is a compound of Formula (8):

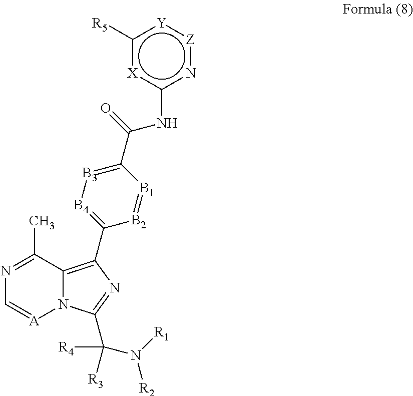

or a pharmaceutically acceptable salt, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
B₂ is N or C(R₈);
B₃ is N or C(R₉);
B₄ is N or C(R₁₀);
R₁ is R₁₁C(O), R₁₂S(O), R₁₃SO₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₄)alkyl, (C₁₋₃)alkoxy, (C₃₋₆)cycloalkyl; all alkyl groups of R5 are optionally substituted with one or more halogen; or R₅ is (C₆₋₁₀)aryl or (C₂₋₆)heterocycloalkyl;
R₆ is H or (C₁₋₃)alkyl; or R₅ and R₆ together may form a (C₃₋₇)cycloalkenyl, or (C₂₋₆)heterocycloalkenyl; each optionally substituted with (C₁₋₃)alkyl, or one or more halogen;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₅)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy;
R₁₁ is independently selected from a group consisting of (C₁₋₆)alkyl, (C₂₋₆)alkenyl and (C₂₋₆)alkynyl each alkyl, alkenyl or alkynyl optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl or (C₃₋₇)heterocycloalkyl, or R₁₁ is (C₁₋₃)alkyl-C(O)—S—(C₁₋₃)alkyl; or
R₁₁ is (C₁₋₅)heteroaryl optionally substituted with one or more groups selected from halogen or cyano.
R₁₂ and R₁₃ are independently selected from a group consisting of (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, or (C₃₋₇)heterocycloalkyl; or
(C₁₋₅)heteroaryl optionally substituted with one or more groups selected from halogen or cyano;
R₁₄ is independently selected from a group consisting of halogen, cyano or (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, (C₁₋₅)heteroaryl or (C₃₋₇)heterocycloalkyl;
with the proviso that
   0 to 2 atoms of X, Y, Z can simultaneously be a heteroatom;
   when one atom selected from X, Y is O or S, then Z is a bond and the other atom selected from X, Y cannot be O or S;
   when Z is C or N then Y is C(R₆) or N and X is C or N;
   0 to 2 atoms of B₁, B₂, B₃ and B₄ are N;
      with the terms used having the following meanings:
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₆)alkyl means a branched or unbranched alkyl group having 1-6 carbon atoms, for example methyl, ethyl, propyl, isopropyl, butyl, tert-butyl, n-pentyl and n-hexyl. (C₁₋₅)alkyl groups are preferred, (C₁₋₄)alkyl being most preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined, with (C₁₋₂)alkoxy groups preferred;
(C₂₋₄)alkenyl means a branched or unbranched alkenyl group having 2-4 carbon atoms, such as ethenyl, 2-propenyl, isobutenyl or 2-butenyl;
(C₂₋₆)alkenyl means a branched or unbranched alkenyl group having 2-6 carbon atoms, such as ethenyl, 2-butenyl, and n-pentenyl, with (C₂₋₄)alkenyl groups preferred, and (C₂₋₃)alkenyl groups even more preferred;
(C₂₋₄)alkynyl means a branched or unbranched alkynyl group having 2-4 carbon atoms, such as ethynyl, 2-propynyl or 2-butynyl;
(C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl, with (C₂₋₄)alkynyl groups preferred, and (C₂₋₃)alkynyl groups more preferred;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; preferred groups are piperidine, morpholine, pyrrolidine and piperazine; a most preferred (C₂₋₆)heterocycloalkyl is pyrrolidine; and the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S; preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; even more preferred are piperidine and pyrrolodine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S, wherein the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, and the more preferred (C₁₋₅)heteroaryl is pyrimidyl;
[(C₁₋₄)alkyl]amino means an amino group, monosubstituted with an alkyl group containing 1-4 carbon atoms having the same meaning as previously defined; the preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; the preferred di[(C₁₋₄)alkyl]amino group is dimethylamino; halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; and cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; the preferred (C₂₋₆)heterocycloalkenyl groups are oxycyclohexenyl and azacyclohexenyl groups.
In the above definitions with multifunctional groups, the attachment point is at the last group.
When, in the definition of a substituent, is indicated that “all of the alkyl groups” of said substituent are optionally substituted, this also includes the alkyl moiety of an alkoxy group.
A circle in a ring of Formula (8) indicates that the ring is aromatic.
Depending on the ring formed, the nitrogen, if present in X or Y, may carry a hydrogen.

In a preferred embodiment, the invention relates to a compound according to Formula (8) wherein B₁ is C(R₇); B₂ is C(R₈); B₃ is C(R₉) and B₄ is C(R₁₀).

In other embodiments, the BTK inhibitors include, but are not limited to, those compounds described in International Patent Application Publication No. WO 2013/010869 and U.S. Patent Application Publication No. US 2014/0155406 A1, the disclosures of each of which are specifically incorporated by reference herein.

In an embodiment, the BTK inhibitor is a compound of Formula (9):

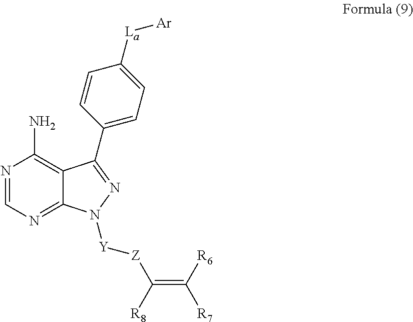

or a pharmaceutically acceptable salt, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O), or NRS(═O)ₓ, where x is 1 or 2;
R⁷ and R are each independently H; or R⁷ and R⁸ taken together form a bond;
R⁶ is H; and
R is H or (C₁₋₆)alkyl.

In a preferred embodiment, the BTK inhibitor is ibrutinib or a pharmaceutically acceptable salt, solvate, hydrate, cocrystal, or prodrug thereof. In a preferred embodiment, the BTK inhibitor is (R)-1-(3-(4-amino-3-(4-phenoxyphenyl)-1H-pyrazolo[3,4-d]pyrimidin-1-yl)piperidin-1-yl)prop-2-en-1-one. In a preferred embodiment, the BTK inhibitor is 1-[(3R)-3-[4-amino-3-(4-phenoxyphenyl)-1H-pyrazolo[3,4-d]pyrimidin-1-yl]piperidin-1-yl]prop-2-en-1-one. In a preferred exemplary embodiment, the BTK inhibitor is (S)-1-(3-(4-amino-3-(4-phenoxyphenyl)-1H-pyrazolo[3,4-d]pyrimidin-1-yl)piperidin-1-yl)prop-2-en-1-one. In a preferred embodiment, the BTK inhibitor has the structure of Formula (10):

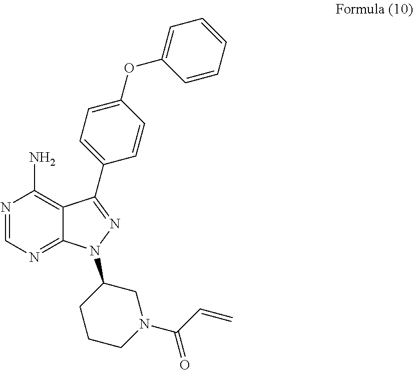 [SMILES: C=CC(=O)N1CCC[C@@H](n2nc(-c3ccc(Oc4ccccc4)cc3)c3c(N)ncnc32)C1]

or an enantiomer thereof, or a pharmaceutically acceptable salt, solvate, hydrate, cocrystal, or prodrug thereof.

In an exemplary embodiment, the BTK inhibitor is a compound of Formula (11):

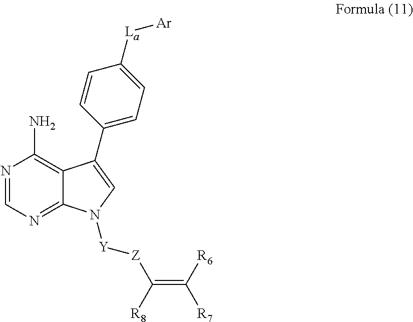

or a pharmaceutically acceptable salt, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O), OS(═O), or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (12):

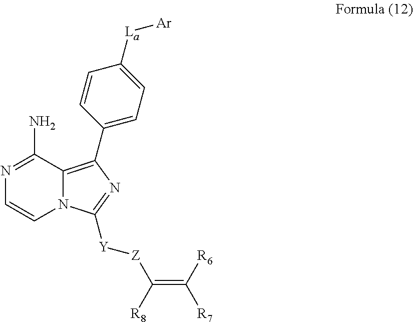

or a pharmaceutically acceptable salt, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (13):

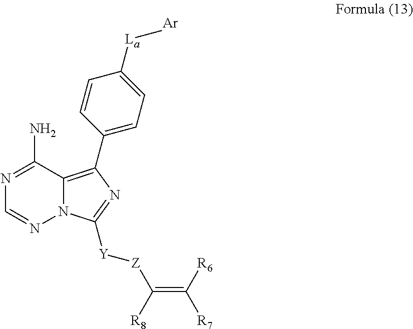

or a pharmaceutically acceptable salt, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound disclosed in U.S. Pat. No. 7,459,554, the disclosure of which is specifically incorporated herein by reference. In an embodiment, the BTK inhibitor is a compound of Formula (14):

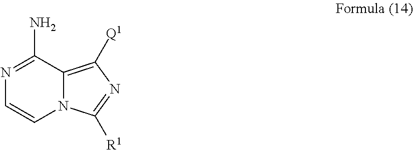

or a pharmaceutically acceptable salt, solvate, hydrate, cocrystal, or prodrug thereof, wherein:
Q¹ is aryl¹, heteroaryl¹, cycloalkyl, heterocyclyl, cycloalkenyl, or heterocycloalkenyl, any of which is optionally substituted by one to five independent G¹ substituents;
   R¹ is alkyl, cycloalkyl, bicycloalkyl, aryl, heteroaryl, aralkyl, heteroaralkyl, heterocyclyl, or heterobicycloalkyl, any of which is optionally substituted by one or more independent G¹¹ substituents;
G¹ and G⁴¹ are each independently halo, oxo, —CF₃, —OCF₃, —OR², —NR²R³(R^{3a})ⱼ₁, —C(O)R², —CO₂R², —CONR²R³, —NO₂, —CN, —S(O)ⱼ₁R², —SO₂NR²R³, NR²(C═O)R³, NR²(C═O)OR³, NR²(C═O)NR²R³, NR²S(O)ⱼ₁R³, —(C═S)OR², —(C═O)SR², —NR²(C═NR³)NR^{2a}R^{3a}, —NR²(C═NR³)OR^{2a}, —NR²(C═NR³)SR^{3a}, —O(C═O)OR², —O(C═O)NR²R³, —O(C═O)SR², —S(C═O)OR², —S(C═O)NR²R³, (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀) alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈) alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, oxo, —CF₃, —OCF₃, —OR²²², —NR²²²R³³³(R³³³a)ⱼ₁ₐ, —C(O)R²²², —CO₂R²²², —CONR²²²R³³³, —NO₂, —CN, —S(O)ⱼ₁ₐR²²², —SO₂NR²²²R³³³, NR²²²(C═O)R³³³, NR²²²(C═O)OR³³³, NR²²²(C═O)NR²²²R³³³, NR²²²S(O)ⱼ₁ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents; or —(X¹)ₙ—(Y′)ₘ—R⁴; or aryl-(C₀₋₁₀)alkyl, aryl-(C₂₋₁₀)alkenyl, or aryl-(C₂₋₁₀) alkynyl, any of which is optionally substituted with one or more independent halo, —CF₃, —OCF₃, —OR²²², —NR²²²R³³³(R^{333a})ⱼ₂ₐ, —C(O)R²²², —CO₂R²²², —CONR²²²R³³³, —NO², —CN, —S(O)ⱼ₂ₐR²²², —SO₂NR²²²R³³³, NR²²²(C═O)R³³³, NR²²²(C═O)OR³³³, NR²²²(C═O)NR²²²R³³³, —NR²²²S(O)ⱼ₂ₐR³³³, —(C═S)OR²²², —(C═O)SR²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²², —S(C═O)OR²²², or —S(C═O)NR²²²R³³³ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR²²², —NR²²², R³³³(R^{333a})ⱼ₃ₐ, —C(O)R²²², —CO₂R²²², —CONR²²²R³³³, —NO₂, —CN, —S(O)ⱼ₃ₐR²²², —SO₂NR²²²R³³³, NR²²²(C═O)R³³³, NR²²²(C═O)OR³³³, NR²²²(C═O)NR²²²R³³³, NR²²²S(O)ⱼ₃ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents;
G¹¹ is halo, oxo, —CF₃, —OCF₃, —OR²¹, —NR²¹R³¹(R^{3a1})ⱼ₄, —C(O)R²¹, —CO₂R²¹, —CONR²¹R³¹, —NO₂, —CN, —S(O)ⱼ₄R²¹, —SO₂NR^{21R31}, NR²¹(C═O)R³¹, NR²¹(C═O)OR³¹, NR²¹(C═O)NR²¹R³¹, NR²¹S(O)ⱼ₄R³¹, —(C═S)OR²¹, —(C═O)SR²¹, —NR²¹ (C═NR³¹)NR^{2a1}R^{3a1}, —NR²¹(C═NR³¹)OR^{2a1}, —NR²¹(C═NR³¹)SR^{3a1}, —O(C═O)OR²¹, —O(C═O)NR²¹R³¹, —O(C═O)SR²¹, —S(C═O)OR²¹, —S(C═O)NR²¹R³¹, —P(O)OR²¹OR³¹, (C₀₋₁₀)alkyl, (C₂₋₁₀)alkenyl, (C₂₋₁₀)alkynyl, (C₁₋₁₀) alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀) alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀) alkyl, cyclo(C₃₋₈)alkyl(C₂₋₁₀)alkenyl, cyclo(C₃₋₈)alkenyl(C₂₋₁₀)alkenyl, cyclo(C₃₋₈) alkyl(C₂₋₁₀) alkynyl, cyclo(C₃₋₈)alkenyl(C₂₋₁₀)alkynyl, heterocyclyl-(C₀₋₁₀)alkyl, heterocyclyl-(C₂₋₁₀) alkenyl, or heterocyclyl-(C₂₋₁₀)alkynyl, any of which is optionally substituted with one or more independent halo, oxo, —CF₃, —OCF₃, —OR²²²¹, —NR²²²¹R³³³¹(R^{333a1}, C(O)R²²²¹, —CO₂R²²²¹, —CONR²²²¹R³³³¹, —NO₂, —CN, —S(O)ⱼ₄ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₄ₐR³³³¹, —(C═S)OR²²²¹, —(C═O)SR²²²¹, —NR²²²¹ (C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R substituents; or aryl-(C₁₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR²²²¹, —NR²²²¹R³³³¹(R^{333a1})ⱼ₅ₐ, —C(O)R²²²¹, —CO₂R²²²¹, —CONR²²²¹R³³³¹, —NO², —CN, —S(O)ⱼ₅ₐR²²²¹, —SO₂NR²²²¹R³³³¹, NR²²²¹(C═O)R³³³¹, NR²²²¹(C═O)OR³³³¹, NR²²²¹(C═O)NR²²²R³³³¹, NR²²²¹S(O)ⱼ₅ₐR³³³¹, (C═S)OR²²²¹, (C═O)SR²²²¹, —NR²²²¹(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹R³³³¹, or —S(C═O)NR²²²¹R³³³¹ substituents; or hetaryl-(C₀₋₁₀) alkyl, hetaryl-(C₂₋₁₀)alkenyl, or hetaryl-(C₂₋₁₀)alkynyl, any of which is optionally substituted with one or more independent halo, —CF₃, —OCF₃, —OR²²²¹, —NR²²²¹R³³³¹(R^{333a1})ⱼ₆ₐ, —C(O)R²²²¹, —CO₂R²²²¹, —CONR²²²¹R³³³¹, —NO₂, —CN, —S(O)ⱼ₆ₐR²²²¹, —SO₂NR²²²¹R³³³¹, NR²²²¹ (C═)R³³³¹, NR²²²¹(C═O)OR³³³¹, NR²²²¹(C═O)NR²²²¹R³³³¹, NR²²²¹S(O)ⱼ₆ₐR³³³¹, —(C═S)OR²²²¹, (C═O)SR²²²¹, —NR²²21(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R³³³¹ substituents; or G¹¹ is taken together with the carbon to which it is attached to form a double bond which is substituted with R⁵ and G¹¹¹;
R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently equal to (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl₍₂₋₁₀₎alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted by one or more G¹¹¹ substituents; or aryl-(C₀₋₁₀)alkyl, aryl-(C₂₋₁₀)alkenyl, or aryl-(C₂₋₁₀)alkynyl, hetaryl-(C₀₋₁₀)alkyl, hetaryl-(C₂₋₁₀)alkenyl, or hetaryl-(C₂₋₁₀)alkynyl, any of which is optionally substituted by one or more G¹¹¹ substituents; or in the case of —NR²R³(R^{3a})ⱼ₁ or —NR²²²R³³³(R^{333a})ⱼ₁ₐ or —NR²²²R³³³(R^{333a})ⱼ₂ₐ or —NR²²²¹R³³³¹(R^{333a1})ⱼ₃ₐ or —NR²²²¹R³³³¹(R^{3331a1})ⱼ₄ₐ or —NR²²²¹R³³³¹(R^{33a1})ⱼ₅ₐ or —NR²²²¹R³³³¹(R^{333a1})ⱼ₆ₐ, R² and R³ or R²²² and R³³³3 or R²²²¹ and R³³³¹ taken together with the nitrogen atom to which they are attached form a 3-10 membered saturated ring, unsaturated ring, heterocyclic saturated ring, or heterocyclic unsaturated ring, wherein said ring is optionally substituted by one or more G¹¹¹ substituents;
X¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C.ident.C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂— —OC(O)N(R⁷)—, —N(R⁷)C(O)N(R⁷)—, —NR⁷C(O)O—, —S(O)N(R)—, —S(O)₂N(R⁷)—, —N(C(O)R⁷)S(O)—, —N(C(O)R⁷)S(O)₂—, —N(R⁷)S(O)N(R⁷)—, —N(R⁷)S(O)₂N(R⁷)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁷)—, —N(R⁷)SO₂N(R⁷)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁷)—, —CH(R⁷)N(C(O)R⁷)—, —CH(R⁷)N(SO₂R⁷)—, —CH(R⁷)O—, —CH(R⁷)S—, —CH(R⁷)N(R⁷)—, —CH(R⁷)N(C(O)R⁷)—, —CH(R⁷)N(C(O)OR⁷)—, —CH(R⁷)N(SO₂R⁷)—, —CH(R⁷)C(═NOR⁷)—, —CH(R⁷)C(O)—, —CH(R⁷)CH(OR⁷)—, —CH(R⁷)C(O)N(R⁷)—, —CH(R⁷)N(R⁷)C(O)—, —CH(R⁷)N(R⁷)S(O)—, —CH(R⁷)N(R⁷)S(O)₂—, —CH(R⁷)OC(O)N(R⁷)—, —CH(R⁷)N(R⁷)C(O)N(R⁷)—, —CH(R⁷)NR⁷C(O)O—, —CH(R⁷)S(O)N(R⁷)—, —CH(R⁷)S(O)₂N(R⁷)—, —CH(R⁷)N(C(O)R⁷)S(O)—, —CH(R⁷)N(C(O)R⁷)S(O)—, —CH(R⁷)N(R⁷)S(O)N(R⁷)—, —CH(R⁷)N(R⁷)S(O)₂N(R⁷)—, —CH(R⁷)C(O)N(R⁷)C(O)—, —CH(R⁷)S(O)N(R⁷)C(O)—, —CH(R⁷)S(O)₂N(R⁷)C(O)—, —CH(R⁷)OS(O)N(R⁷)—, —CH(R⁷)OS(O)₂N(R⁷)—, —CH(R⁷)N(R⁷)S(O)O—, —CH(R⁷)N(R⁷)S(O)₂O—, —CH(R⁷)N(R⁷)S(O)C(O)—, —CH(R⁷)N(R⁷)S(O)₂C(O)—, —CH(R⁷)SON(C(O)R⁷)—, —CH(R⁷)SO₂N(C(O)R⁷)—, —CH(R⁷)N(R⁷)SON(R⁷)—, —CH(R⁷)N(R⁷)SO₂N(R⁷)—, —CH(R⁷)C(O)O—, —CH(R⁷)N(R⁷)P(OR⁸)O—, —CH(R⁷)N(R⁷)P(OR⁸)—, —CH(R⁷)N(R⁷)P(O)(OR⁸)O—, —CH(R⁷)N(R⁷)P(O)(OR⁸)—, —CH(R⁷)N(C(O)R⁷)P(OR)O—, —CH(R⁷)N(C(O)R⁷)P(OR⁸)—, —CH(R⁷)N(C(O)R⁷)P(O)(OR⁸)O—, or —CH(R⁷)N(C(O)R⁷)P(OR⁸)—;
or X¹ and Y¹ are each independently represented by one of the following structural formulas:

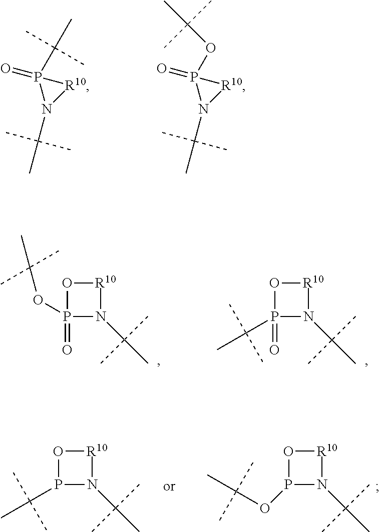

R¹⁰, taken together with the phosphinamide or phosphonamide, is a 5-, 6-, or 7-membered aryl, heteroaryl or heterocyclyl ring system;
R⁵, R⁶, and G¹¹¹ are each independently a (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or aryl-(C₀₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, NR⁷⁷(C═NR⁸⁷)NR⁷⁸R⁸⁸, NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷R⁸⁷, or —S(C═O)NR⁷⁷R⁸⁷ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR⁷⁷, —NR⁷⁷R⁸⁷, C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)N⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or R⁵ with R⁶ taken together with the respective carbon atom to which they are attached, form a 3-10 membered saturated or unsaturated ring, wherein said ring is optionally substituted with R⁶⁹; or R⁵ with R⁶ taken together with the respective carbon atom to which they are attached, form a 3-10 membered saturated or unsaturated heterocyclic ring, wherein said ring is optionally substituted with R⁶⁹;
R⁷ and R⁸ are each independently H, acyl, alkyl, alkenyl, aryl, heteroaryl, heterocyclyl or cycloalkyl, any of which is optionally substituted by one or more G¹¹¹ substituents;
R⁴ is H, alkyl, alkenyl, alkynyl, aryl, heteroaryl, cycloalkyl, heterocyclyl, cycloalkenyl, or heterocycloalkenyl, any of which is optionally substituted by one or more G⁴¹ substituents;
R⁶⁹ is equal to halo, —OR⁷⁸, —SH, —NR⁷⁸R⁸⁸, —CO₂R⁷⁸, —CONR⁷⁸R⁸⁸, —NO₂, —CN, —S(O)ⱼ₈R⁷⁸, —SO₂NR⁷⁸R⁸⁸, (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸, or —NR⁷⁷⁸R⁸⁸⁸ substituents; or mono(C₁₋₆alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or in the case of —NR⁷⁸R⁸⁸, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, (C₁₋₁₀)alkoxy, —SO₂NR⁷⁷⁸R⁸⁸⁸, or —NR⁷⁷⁸R⁸⁸⁸ substituents;
R⁷⁷, R⁷⁸, R⁸⁷, R⁸⁸, R⁷⁷⁸, and R⁸⁸⁸ are each independently (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxyC₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃-g)alkenyl(C₂₋₁₀)alkynyl, heterocyclyl-(C₀₋₁₀)alkyl, heterocyclyl-(C₂₋₁₀)alkenyl, heterocyclyl-(C₂₋₁₀)alkynyl, (C₁₋₁₀)alkylcarbonyl, (C₂₋₁₀)alkenylcarbonyl, (C₂₋₁₀)alkynylcarbonyl, (C₁₋₁₀)alkoxycarbonyl, (C₁₋₁₀)alkoxycarbonyl(C₁₋₁₀)alkyl, mono(C₁₋₆)alkylaminocarbonyl, di(C₁₋₆)alkylaminocarbonyl, mono(aryl)aminocarbonyl, di(aryl)aminocarbonyl, or (C₁₋₁₀)alkyl(aryl)aminocarbonyl, any of which is optionally substituted with one or more independent halo, cyano, hydroxy, nitro, (C₁₋₁₀)alkoxy, —SO₂N((C₀₋₄)alkyl)((C₀₋₄)alkyl), or —N((C₀₋₄)alkyl)((C₀₋₄)alkyl) substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₁₀)alkyl), —SO₂N((C₀₋₄)alkyl)((C₀₋₄)alkyl), or —N((C₀₋₄)alkyl)((C₀₋₄)alkyl) substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((CO—₄)alkyl)((C₀₋₄)alkyl), or —N((C₀₋₄)alkyl)((C₀₋₄)alkyl) substituents; or mono((C₁₋₆)alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((C₀₋₄)alkyl)((C₀₋₄)alkyl), or —N((C₀₋₄)alkyl)((C₀₋₄)alkyl) substituents; and
n, m, j1, j1a, j2a, j3a, j4, j4a, j5a, j6a, j7, and j8 are each independently equal to 0, 1, or 2.

In an embodiment, the BTK inhibitor is a compound selected from the structures disclosed in U.S. Pat. Nos. 8,450,335 and 8,609,679, and U.S. Patent Application Publication Nos. 2010/0029610 A1, 2012/0077832 A1, 2013/0065879 A1, 2013/0072469 A1, and 2013/0165462 A1, the disclosures of which are incorporated by reference herein. In an embodiment, the BTK inhibitor is a compound of Formula (15) or Formula (16):

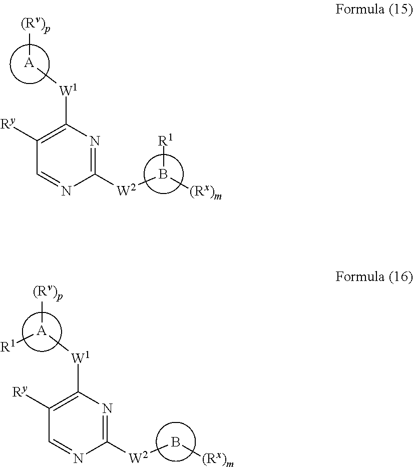

or a pharmaceutically acceptable salt, solvate, hydrate, cocrystal, or prodrug thereof, wherein:
Ring A is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is a warhead group;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²—, —N(R²)C(O)—, —C(O)N(R²)—, —N(R²)SO₂—, —SO₂N(R²)—, —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered saturated, partially unsaturated, or aromatic fused ring, or:
R² and R^{y} are taken together with their intervening atoms to form an optionally substituted 4-7 membered partially unsaturated or aromatic fused ring;
m and p are independently 0-4; and
R^{x} and R^{v} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein q is 1-4; or:
R^{x} and R¹ when concurrently present on Ring B are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic.

In an embodiment, the BTK inhibitor is a compound of Formula (15) or Formula (16), wherein:
Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is -L-Y, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, SO₂N(R)—, —O—, —C(O)—, —OC(O)—, —C(O)O—, —S—, —SO—, —SO₂—, —C(S)—, —C(═NR)—, —N═N—, or —C(═N₂)—;
Y is hydrogen, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 groups independently selected from -Q-Z, oxo, NO₂, halogen, CN, or C₁₋₆ aliphatic, wherein:
Q is a covalent bond or a bivalent C₁₋₆ saturated or unsaturated, straight or branched, hydrocarbon chain, wherein one or two methylene units of Q are optionally and independently replaced by —NR—, —S—, —O—, —C(O)—, —SO—, or —SO₂—; and
Z is hydrogen or C₁₋₆ aliphatic optionally substituted with oxo, halogen, or CN;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²—, —N(R²)C(O)—, —C(O)N(R²)—, —N(R²)SO₂—, —SO₂N(R²), —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered partially unsaturated or aromatic fused ring; or
R² and R^{y} are taken together with their intervening atoms to form a 4-6 membered saturated, partially unsaturated, or aromatic fused ring;
m and p are independently 0-4; and
R^{x} and R^{v} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein R is independently selected from the group consisting of hydrogen, cycloalkyl, alkenyl, cycloalkenyl, alkynyl, aryl, heteroaryl, and heterocycly; or:
R^{x} and R¹ when concurrently present on Ring B are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic.

As defined generally above, Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In preferred embodiments, Ring A is an optionally substituted phenyl group. In some embodiments, Ring A is an optionally substituted naphthyl ring or an optionally substituted bicyclic 8-10 membered heteroaryl ring having 1-4 heteroatoms independently selected from nitrogen, oxygen, or sulfur. In certain other embodiments, Ring A is an optionally substituted 3-7 membered carbocyclic ring. In yet other embodiments, Ring A is an optionally substituted 4-7 membered heterocyclic ring having 1-3 heteroatoms independently selected from nitrogen, oxygen, or sulfur. In preferred embodiments, Ring B is an optionally substituted phenyl group.

In certain embodiments, Ring A in Formula (15) or Formula (16) is substituted as defined herein. In some embodiments, Ring A is substituted with one, two, or three groups independently selected from halogen, R^{o}, or —(CH₂)₀₋₄OR^{o}, or —O(CH₂)₀₋₄R^{o}, wherein each R^{o} is independently selected from the group consisting of cycloalkyl, alkenyl, cycloalkenyl, alkynyl, aryl, heteroaryl, and heterocyclyl. Exemplary substituents on Ring A include Br, I, Cl, methyl, —CF₃, —C≡CH, —OCH₂phenyl, —OCH₂(fluorophenyl), or —OCH₂pyridyl.

In a preferred embodiment, the BTK inhibitor is CC-292 (also known as AVL-292), or a pharmaceutically acceptable salt, solvate, hydrate, cocrystal, or prodrug thereof, preferably a hydrochloride salt or a besylate salt thereof. In a preferred embodiment, the BTK inhibitor is a compound of Formula (17):

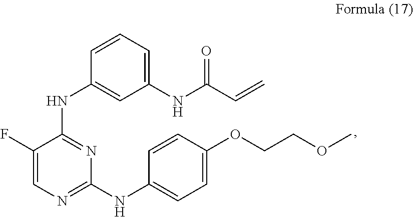 [SMILES: C=CC(=O)Nc1cccc(Nc2nc(Nc3ccc(OCCOC)cc3)ncc2F)c1]

which is N-(3-((5-fluoro-2-((4-(2-methoxyethoxy)phenyl)amino)pyrimidin-4-yl)amino)phenyl)acrylamide, or a pharmaceutically acceptable salt, solvate, hydrate, cocrystal, or prodrug thereof, or in an exemplary embodiment is a hydrochloride salt or a besylate salt thereof. The preparation of this compound is described in U.S. Patent Application Publication No. 2010/0029610 A1 at Example 20, the disclosure of which is incorporated by reference herein. The preparation of the besylate salt of this compound is described in U.S. Patent Application Publication No. 2012/0077832 A1, the disclosure of which is incorporated by reference herein. In an embodiment, the BTK inhibitor is a compound selected from the structures disclosed in U.S. Patent Application Publication No. 2010/0029610 A1 or No. 2012/0077832 A1, the disclosures of which are incorporated by reference herein.

In a preferred embodiment, the BTK inhibitor is N-(3-((5-fluoro-2-((4-(2-methoxyethoxy)phenyl)amino)pyrimidin-4-yl)amino)phenyl)acrylamide or a pharmaceutically acceptable salt, solvate, hydrate, cocrystal, or prodrug thereof, or a hydrochloride salt thereof. The preparation of this compound is described in U.S. Patent Application Publication Nos. 2010/0029610 A1 and 2012/0077832 A1, the disclosure of which is incorporated by reference herein.

In a preferred embodiment, the BTK inhibitor is (N-(3-(5-fluoro-2-(4-(2-methoxyethoxy)phenylamino)pyrimidin-4-ylamino)phenyl)acrylamide), or a pharmaceutically acceptable salt, solvate, hydrate, cocrystal, or prodrug thereof, or preferably a besylate salt thereof. The preparation of this compound is described in U.S. Patent Application Publication No. 2010/0029610 A1 at Example 20, the disclosure of which is incorporated by reference herein. The preparation of its besylate salt is described in U.S. Patent Application Publication No. 2012/0077832 A1, the disclosure of which is incorporated by reference herein.

In an embodiment, the BTK inhibitor is a compound of Formula (18):

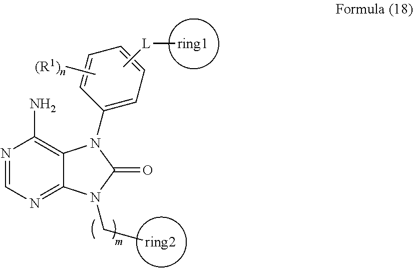

or a pharmaceutically acceptable salt, hydrate, solvate, cocrystal, or prodrug thereof, wherein
L represents (1) —O—, (2) —S—, (3) —SO—, (4) —SO₂— (5) —NH—, (6) —C(O)—, (7) —CH₂O—, (8) —O—CH₂—, (9) —CH₂—, or (10) —CH(OH)—;
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1 represents a 4- to 7-membered cyclic group, which may be substituted by from one to five substituents each independently selected from the group consisting of (1) halogen atoms, (2) C₁₋₄ alkyl groups, (3) C₁₋₄ alkoxy groups, (4) nitrile, (5) C₁₋₄ haloalkyl groups, and (6) C₁₋₄ haloalkoxy groups, wherein when two or more substituents are present on ring1, these substituents may form a 4- to 7-membered cyclic group together with the atoms in ring1 to which these substituents are bound;
ring2 represents a 4- to 7-membered saturated heterocycle, which may be substituted by from one to three —K—R²; K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR¹;
R³ and R⁴ each independently represent (1) a hydrogen atom, or (2) a C₁₋₄ alkyl group which may be substituted by OR⁹ or CONR¹⁰R¹R; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group;
n represents an integer from 0 to 4;
m represents an integer from 0 to 2; and
when n is two or more, the R¹'s may be the same as each other or may differ from one another).

In an exemplary embodiment, the BTK inhibitor is a compound of Formula (19):

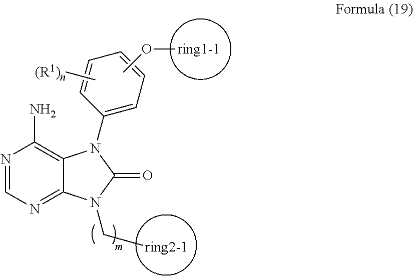

or a pharmaceutically acceptable salt, hydrate, solvate, cocrystal, or prodrug thereof, wherein
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1 represents a benzene, cyclohexane, or pyridine ring, each of which may be substituted by from one to five substituents each independently selected from the group consisting of (1) halogen atoms, (2) C₁₋₄ alkyl groups, (3) C₁₋₄ alkoxy groups, (4) nitrile, (5) CF₃;
ring2 represents a 4- to 7-membered nitrogenous saturated heterocycle, which may be substituted by from one to three —K—R²; wherein K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR⁸;
R³ and R⁴ each independently represent (1) a hydrogen atom, or (2) a C₁₋₄ alkyl group which may be substituted by OR⁹ or CONR¹⁰R¹¹; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group;
n represents an integer from 0 to 4;
m represents an integer from 0 to 2; and
when n is two or more, the R¹'s may be the same as each other or may differ from one another).

In a preferred embodiment, the BTK inhibitor is a compound of Formula (20):

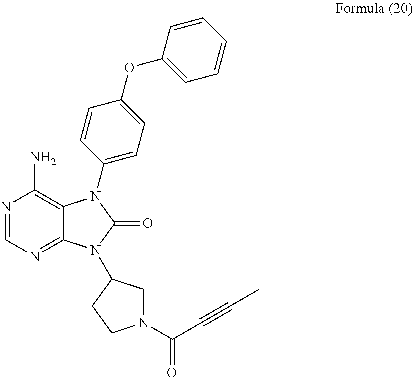 [SMILES: CC#CC(=O)N1CCC(n2c(=O)n(-c3ccc(Oc4ccccc4)cc3)c3c(N)ncnc32)C1]

or a pharmaceutically acceptable salt, solvate, hydrate, cocrystal, or prodrug thereof, preferably a hydrochloride salt thereof. The preparation of this compound is described in International Patent Application Publication No. WO 2013/081016 A1 and U.S. Patent Application Publication No. US 2014/0330015 A1, the disclosure of which is incorporated by reference herein. In an embodiment, the BTK inhibitor is 6-amino-9-(1-(but-2-ynoyl)pyrrolidin-3-yl)-7-(4-phenoxyphenyl)-7,9-dihydro-8H-purin-8-one or a pharmaceutically acceptable salt, solvate, hydrate, cocrystal, or prodrug thereof, or preferably a hydrochloride salt thereof. In an embodiment, the BTK inhibitor is 6-amino-9-[(3S)-1-(2-butynoyl)-3-pyrrolidinyl]-7-(4-phenoxyphenyl)-7,9-dihydro-8H-purin-8-one or a pharmaceutically acceptable salt, solvate, hydrate, cocrystal, or prodrug thereof, or a hydrochloride salt thereof.

The R-enantiomer of Formula (20) is also known as ONO-4059, and is given by Formula (21). In a preferred embodiment, the BTK inhibitor is a compound of Formula (21):

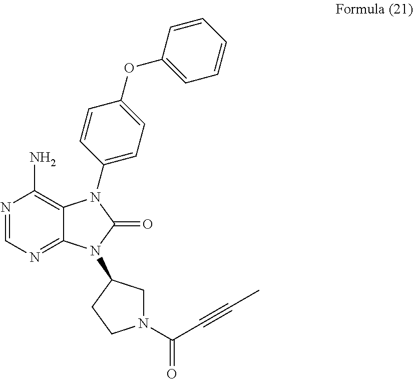 [SMILES: CC#CC(=O)N1CC[C@@H](n2c(=O)n(-c3ccc(Oc4ccccc4)cc3)c3c(N)ncnc32)C1]

or a pharmaceutically acceptable salt, solvate, hydrate, cocrystal, or prodrug thereof, preferably a hydrochloride salt thereof.

In an embodiment, the BTK inhibitor is 6-amino-9-[(3R)-1-(2-butynoyl)-3-pyrrolidinyl]-7-(4-phenoxyphenyl)-7,9-dihydro-8H-purin-8-one or a pharmaceutically acceptable salt, solvate, hydrate, cocrystal, or prodrug thereof, preferably a hydrochloride salt thereof.

The preparation of Formula (21) is described in International Patent Application Publication No. WO 2013/081016 A1, the disclosure of which is incorporated by reference herein. In brief, the BTK inhibitor of Formula (21) can be prepared by the following procedure.

Step 1: A solution of dibenzylamine (10.2 g) in dichloromethane (30 mL) is dripped into a solution of 4,6-dichloro-5-nitropyrimidine (10 g) in dichloromethane (70 mL) on an ice bath. Then triethylamine (14.4 mL) is added, and the mixture is stirred for 1 hour. Water is added to the reaction mixture, the organic layer is washed with a saturated aqueous sodium chloride solution and dried over anhydrous sodium sulfate, and the solvent is concentrated under reduced pressure to obtain N,N-dibenzyl-6-chloro-5-nitropyrimidine-4-amine (19.2 g).

Step 2: The compound prepared in Step 1 (19 g) and tert-butyl (3R)-3-aminopyrrolidine-1-carboxylate (10.5 g) are dissolved in dioxane (58 mL). Triethylamine (8.1 mL) is added, and the mixture is stirred for 5 hours at 50° C. The reaction mixture is returned to room temperature, the solvent is distilled off, water is added, and extraction is performed with ethyl acetate. The organic layer is washed with saturated aqueous sodium chloride solution, then dried over anhydrous sodium sulfate, and the solvent is distilled off. The residue is purified by silica gel column chromatography to obtain tert-butyl (3R)-3-{[6-(dibenzylamino)-5-nitropyrimidin-4-yl]amino}pyrrolidine-1-carboxylate (27.0 g).

Step 3: An ethyl acetate (360 mL) solution of the compound prepared in Step 2 (17.5 g) is dripped into a mixture of zinc (23.3 g) and a 3.0 M aqueous ammonium chloride solution (11.4 g) on an ice bath, and the temperature is immediately raised to room temperature. After stirring for 2 hours, the reaction mixture is filtered through CELITE and the solvent is distilled off. The residue is purified by silica gel column chromatography to obtain tert-butyl (3R)-3-[5-amino-6-(dibenzylamino)pyrimidin-4-yl]amino pyrrolidine-1-carboxylate (12.4 g).

Step 4: The compound prepared in Step 3 (8.4 g) and 1,1′-carbonyl diimidazole (5.9 g) are dissolved in tetrahydrofuran (120 mL) and the solution is stirred for 15 hours at 60° C. The solvent is distilled off from the reaction mixture, water is added, and extraction with ethyl acetate is performed. The organic layer is washed with saturated aqueous sodium chloride solution, dried over anhydrous sodium sulfate, and the solvent is distilled off. The residue is purified by silica gel column chromatography to obtain tert-butyl (3R)-3-[6-(dibenzylamino)-8-oxo-7,8-dihydro-9H-purin-9-yl]pyrrolidin-1-carboxylate (7.8 g).

Step 5: The compound prepared in Step 4 (7.8 g) is dissolved in methanol (240 mL) and ethyl acetate (50 mL), 20% Pearlman's catalyst (Pd(OH)₂/C) (8.0 g, 100 wt %) is added, hydrogen gas replacement is carried out, and stirring is performed for 7.5 hours at 60° C. The reaction mixture is filtered through CELITE and the solvent is distilled off to obtain tert-butyl (3R)-3-(6-amino-8-oxo-7,8-dihydro-9H-purin-9-yl)pyrrolidine-1-carboxylate (5.0 g).

Step 6: At room temperature p-phenoxy phenyl boronic acid (2.1 g), copper(II) acetate (1.48 g), molecular sieve 4A (2.5 g), and pyridine (0.82 mL) are added to a dichloromethane suspension (200 mL) of the compound prepared in Step 5 (2.5 g), followed by stirring for 21 hours. The reaction mixture is filtered through CELITE and the residue is purified by silica gel column chromatography to obtain tert-butyl (3R)-3-[6-amino-8-oxo-7-(4-phenoxyphenyl)-7,8-dihydro-9H-purin-9-yl]pyrrolidine-1-carboxylate (1.3 g).

Step 7: At room temperature 4 N HCl/dioxane (13 mL) is added to a methanol (13 mL) suspension of the compound prepared in Step 6 (1.3 g 2.76 mmol, 1.0 equivalent), and the mixture is stirred for 1 hour. The solvent is then distilled off to obtain (3R)-6-amino-9-pyrrolidin-3-yl-7-(4-phenoxyphenyl)-7,9-dihydro-8H-purin-8-one dihydrochloride (1.5 g).

Step 8: After 2-butylnoic acid (34 mg), 1-ethyl-3-(3-dimethylaminopropyl) carbodiimide hydrochloride (EDC) (78 mg), 1-hydroxybenzotriazole (HOBt) (62 mg), and triethylamine (114 mL) are added to a solution of the compound prepared in Step 7 (100 mg) in dimethyl formamide (3 mL), the mixture is stirred at room temperature for 3 hours. Water is added to the reaction mixture and extraction with ethyl acetate is performed. The organic layer is washed with saturated sodium carbonate solution and saturated aqueous sodium chloride solution, then dried over anhydrous sodium sulfate, and the solvent is distilled off. The residue is purified by thin layer chromatography (dichloromethane:methanol:28% ammonia water=90:10:1) to obtain 6-amino-9-[(3R)-1-(2-butynoyl)-3-pyrrolidinyl]-7-(4-phenoxyphenyl)-7,9-dihydro-8H-purin-8-one (Formula (21)) (75 mg).

The hydrochloride salt of the compound of Formula (21) can be prepared as follows: 6-amino-9-[(3R)-1-(2-butynoyl)-3-pyrrolidinyl]-7-(4-phenoxyphenyl)-7,9-dihydro-8H-purin-8-one (3.0 g) (which may be prepared as described above) is placed in a 300 mL 3-neck pear-shaped flask, ethyl acetate (30 mL) and 1-propanol (4.5 mL) are added, and the external temperature is set at 70° C. (internal temperature 61° C.). After it is confirmed that the compound prepared in Step 8 has dissolved completely, 10% HCl/methanol (3.5 mL) is added, and after precipitation of crystals is confirmed, the crystals are ripened by the following sequence: external temperature 70° C. for 30 min, external temperature 60° C. for 30 min, external temperature 50° C. for 60 min, external temperature 40 OC for 30 min, room temperature for 30 min, and an ice bath for 30 min. The resulting crystals are filtered, washed with ethyl acetate (6 mL), and dried under vacuum at 50° C. to obtain white crystals of 6-amino-9-[(3R)-1-(2-butynoyl)-3-pyrrolidinyl]-7-(4-phenoxyphenyl)-7,9-dihydro-8H-purin-8-one hydrochloride (2.76 g).

In an embodiment, the BTK inhibitor is a compound selected from the structures disclosed in U.S. Patent Application Publication No. US 2014/0330015 A1, the disclosure of which is incorporated by reference herein.

In an embodiment, the BTK inhibitor is a compound of Formula (22):

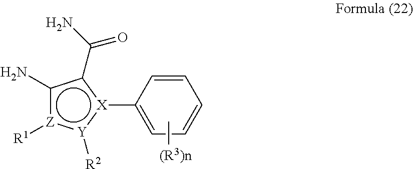

or a pharmaceutically acceptable salt, solvate, hydrate, cocrystal, or prodrug thereof, wherein:
X—Y—Z is N—C—C and R² is present, or C—N—N and R² is absent;
R¹ is a 3-8 membered, N-containing ring, wherein the N is unsubstituted or substituted with R⁴;
R² is H or lower alkyl, particularly methyl, ethyl, propyl or butyl; or
R¹ and R² together with the atoms to which they are attached, form a 4-8 membered ring, preferably a 5-6 membered ring, selected from cycloalkyl, saturated or unsaturated heterocycle, aryl, and heteroaryl rings unsubstituted or substituted with at least one substituent L-R⁴;
R³ is in each instance, independently halogen, alkyl, S-alkyl, CN, or OR⁵;
n is 1, 2, 3, or 4, preferably 1 or 2;
L is a bond, NH, heteroalkyl, or heterocyclyl;
R⁴ is COR′, CO₂R′, or SO₂R′, wherein R′ is substituted or unsubstituted alkyl, substituted or unsubstituted alkenyl, substituted or unsubstituted alkynyl;
R⁵ is H or unsubstituted or substituted heteroalkyl, alkyl, cycloalkyl, saturated or unsaturated heterocyclyl, aryl, or heteroaryl.

In some embodiments, the BTK inhibitor is one of the following particular embodiments of Formula (22):
X—Y—Z is C—N—N and R² is absent; and R¹ is 3-8 membered, N-containing ring, N-substituted with R⁴;
X—Y—Z is N—C—C and R² is present, R¹ is 3-8 membered, N-containing ring, N-substituted with R⁴; and R² is H or lower alkyl;
X—Y—Z is N—C—C and R² is present; and R¹ and R² together with the atoms to which they are attached, form a 4-8 membered ring selected from cycloalkyl, saturated or unsaturated heterocycle, aryl, and heteroaryl rings unsubstituted or substituted with at least one substituent L-R⁴, wherein preferred rings of R¹ and R² are 5-6-membered, particularly dihydropyrrole, tetrahydropyridine, tetrahydroazepine, phenyl, or pyridine;
X—Y—Z is N—C—C and R² is present; and R¹ and R² together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond;
R¹ is piperidine or azaspiro[3.3]heptane, preferably N-substituted with R⁴;
R⁴ is COR′ or SO₂R′, particularly wherein R′ is substituted or unsubstituted alkenyl, particularly substituted or unsubstituted ethenyl; or
R⁵ is unsubstituted or substituted alkyl or aryl, particularly substituted or unsubstituted phenyl or methyl, such as cyclopropyl-substituted methyl with or tetrabutyl-substituted phenyl.

In some embodiments, the BTK inhibitor is one of the following particular embodiments of Formula (22):
R¹ is piperidine or azaspiro[3.3]heptane, N-substituted with R⁴, wherein R⁴ is H, COR′ or SO₂R′, and R′ is substituted or unsubstituted alkenyl, particularly substituted or unsubstituted ethenyl;
R³ is —OR⁵, R⁵ is phenyl, and n is 1;
R¹ and R², together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond; R³ is —OR⁵; n is 1;
R⁴ is COR′, and R′ is ethenyl; and R⁵ is phenyl; and
X—Y—Z is C—N—N and R² is absent; R′ is piperidine, N-substituted with R⁴; R³ is —OR; n is 1; R⁴ is COR′, and R′ is unsubstituted or substituted alkenyl, particularly ethenyl; and R⁵ is substituted or unsubstituted aryl, particularly phenyl.

In some embodiments, the BTK inhibitor is a compound selected from the group consisting of Formula (23), Formula (24), or Formula (25):

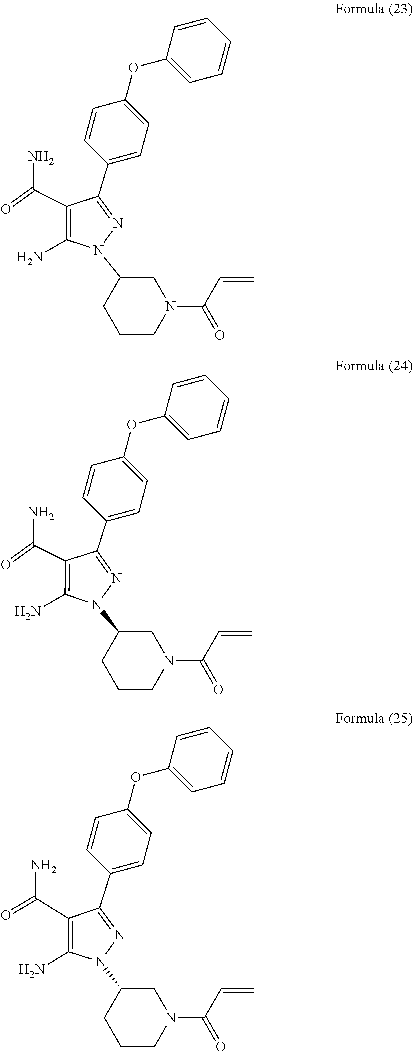

or a pharmaceutically acceptable salt, solvate, hydrate, cocrystal, or prodrug thereof. Formula (24) is also known as BGB-3111. The preparation of these compounds is described in International Patent Application Publication No. WO 2014/173289 A1 and U.S. Patent Application Publication No. US 2015/0005277 A1, the disclosure of which is incorporated by reference herein.

In brief, the BTK inhibitor of Formula (23) can be prepared by the following procedure.

### Step 1. Preparation of 2-(hydroxy(4-phenoxyphenyl)methylene)malononitrile

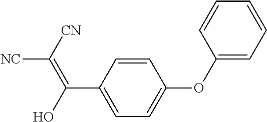 [SMILES: N#CC(C#N)=C(O)c1ccc(Oc2ccccc2)cc1]

A solution of 4-phenoxybenzoic acid (300 g, 1.4 mol) in SOCl₂ (1.2 L) is stirred at 80° C. under N₂ for 3 hours. The mixture is concentrated in vacuum to give the intermediate (315 g) which is used for next step without further purification.

To a solution of propanedinitrile (89.5 g, 1355 mmol) and N,N-diisopropylethylamine (DIEA) (350 g, 2710 mmol) in THF (800 mL) is added dropwise a solution of the intermediate (315 g) in toluene (800 mL) at 0-5° C. over 2 hours. The resultant mixture is allowed to warm to RT and stirred for 16 hours. The reaction is quenched with water (2.0 L) and extracted with of EA (2.0 L×3). The combined organic layers are washed with 1000 mL of 3 N HCl aqueous solution, brine (2.0 L×3), dried over Na₂SO₄ and concentrated to give the crude product (330 g, 93%).

### Step 2. Preparation of 2-(methoxy(4-phenoxyphenyl)methylene)malononitrile

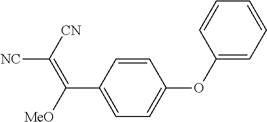 [SMILES: COC(=C(C#N)C#N)c1ccc(Oc2ccccc2)cc1]

A solution of 2-(hydroxy(4-phenoxyphenyl)methylene)malononitrile (50 g, 190.8 mmol) in CH(OMe₃) (500 mL) is heated to 75° C. for 16 hours. Then the mixture is concentrated to a residue and washed with MeOH (50 mL) to give 25 g (47.5%) of 2-(methoxy(4-phenoxyphenyl)methylene)malononitrile as a yellow solid.

### Step 3. Preparation of 5-amino-3-(4-phenoxyphenyl)-1H-pyrazole-4-carbonitrile

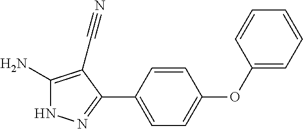 [SMILES: N#Cc1c(-c2ccc(Oc3ccccc3)cc2)n[nH]c1N]

To a solution of 2-(methoxy(4-phenoxyphenyl)methylene)malononitrile (80 g, 290 mmol) in ethanol (200 mL) is added hydrazine hydrate (20 mL). The mixture is stirred at RT for 16 hours then is concentrated to give the crude product and washed with MeOH (30 mL) to afford 55 g (68.8%) of 5-amino-3-(4-phenoxyphenyl)-1H-pyrazole-4-carbonitrile as an off-white solid.

### Step 4. Preparation of tert-butyl 3-(tosyloxy)piperidine-1-carboxylate

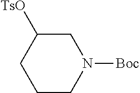 [SMILES: Cc1ccc(S(=O)(=O)OC2CCCN(C(=O)OC(C)(C)C)C2)cc1]

wherein “Boc” represents a tert-butyloxycarbonyl protecting group.

To a solution of tert-butyl 3-hydroxypiperidine-1-carboxylate (1.05 g, 5.0 mmol) in pyridine (8 mL) is added TsCl (1.425 g, 7.5 mmol). The mixture is stirred at RT under N₂ for two days. The mixture is concentrated and partitioned between 100 mL of EA and 100 mL of HCl (1 N) aqueous solution. The organic layer is separated from aqueous layer, washed with saturated NaHCO₃ aqueous solution (100 mL×2), brine (100 mL×3) and dried over Na₂SO₄. The organic layer is concentrated to afford 1.1 g (60%) of tert-butyl 3-(tosyloxy)piperidine-1-carboxylate as a colorless oil.

### Step 5. Preparation of tert-butyl 3-(5-amino-4-cyano-3-(4-phenoxyphenyl)-1H-pyrazol-1-yl)piperidine-1-carboxylate

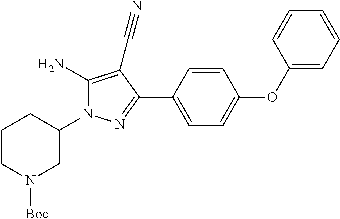 [SMILES: CC(C)(C)OC(=O)N1CCCC(n2nc(-c3ccc(Oc4ccccc4)cc3)c(C#N)c2N)C1]

To a solution of tert-butyl 3-(tosyloxy)piperidine-1-carboxylate (355 mg, 1.0 mmol) and 5-amino-3-(4-phenoxyphenyl)-1H-pyrazole-4-carbonitrile (276 mg, 1.0 mmol) in 5 mL of DMF is added Cs₂CO₃ (650 mg, 2.0 mmol). A tosyloxy leaving group is employed in this reaction. The mixture is stirred at RT for 16 hours, 75° C. for 3 hours and 60 OC for 16 hours. The mixture is concentrated washed with brine (100 mL×3) and dried over Na₂SO₄. The material is concentrated and purified by chromatography column on silica gel (eluted with petroleum ether/ethyl acetate=3/1) to afford 60 mg (13%) of tert-butyl 3-(5-amino-4-cyano-3-(4-phenoxyphenyl)-1H-pyrazol-1-yl)piperidine-1-carboxylate as a yellow oil.

### Step 6. Preparation of tert-butyl 3-(5-amino-4-carbamoyl-3-(4-phenoxyphenyl)-1H-pyrazol-1-yl)piperidine-1-carboxylate

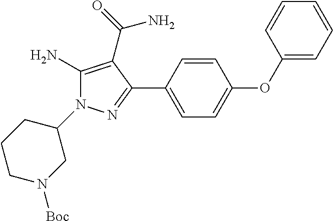 [SMILES: CC(C)(C)OC(=O)N1CCCC(n2nc(-c3ccc(Oc4ccccc4)cc3)c(C(N)=O)c2N)C1]

To a solution of tert-butyl 3-(5-amino-4-cyano-3-(4-phenoxyphenyl)-1H-pyrazol-1-yl)piperidine-1-carboxylate (100 mg, 0.22 mmol) in DMSO (2 mL) and ethanol (2 mL) was added the solution of NaOH (200 mg, 5 mmol) in water (1 mL) and H₂O₂ (1 mL). The mixture is stirred at 60° C. for 15 min and concentrated to remove EtOH, after which 10 mL of water and 50 mL of ethyl acetate are added. The organic layer is separated from aqueous layer, washed with brine (30 mL×3) and dried over Na₂SO₄. After concentration, 50 mg of residue is used directly in the next step, wherein 50 mg of residue is purified by pre-TLC (eluted with petroleum ether/ethyl acetate=1/1) to afford 12 mg (30%) of tert-butyl 3-(5-amino-4-carbamoyl-3-(4-phenoxyphenyl)-1H-pyrazol-1-yl)piperidine-1-carboxylate as a white solid.

### Step 7. Preparation of 5-amino-3-(4-phenoxyphenyl)-1-(piperidin-3-yl)-1H-pyrazole-4-carboxamide

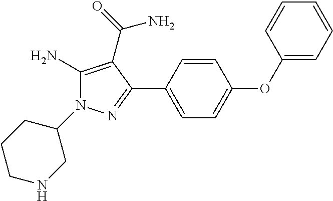 [SMILES: NC(=O)c1c(-c2ccc(Oc3ccccc3)cc2)nn(C2CCCNC2)c1N]

To a solution of tert-butyl 3-(5-amino-4-carbamoyl-3-(4-phenoxyphenyl)-1H-pyrazol-1-yl)piperidine-1-carboxylate (50 mg, 0.11 mmol) in ethyl acetate (1 mL) is added concentrated HCl (0.75 mL). The mixture is stirred at RT for 1 hour. Then saturated NaHCO₃ is added until pH>7, followed by ethyl acetate (50 mL). The organic layer is separated from aqueous layer, washed with brine (50 mL×3) and dried over Na₂SO₄. The resulting product is concentrated and purified by Pre-TLC (eluted with dichloromethane/MeOH/NH₃—H₂O=5/1/0.01) to afford 10 mg (25%) of 5-amino-3-(4-phenoxyphenyl)-1-(piperidin-3-yl)-1H-pyrazole-4-carboxamide as a white solid.

### Step 8. Preparation of 1-(1-acryloylpiperidin-3-yl)-5-amino-3-(4-phenoxyphenyl)-1H-pyrazole-4-carboxamide

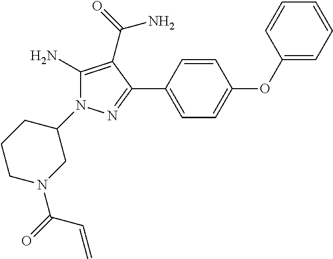 [SMILES: C=CC(=O)N1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (63 mg, 0.17 mmol) in dichloromethane (4 mL) is added pyridine (27 mg, 0.34 mmol). Then a solution of acryloyl chloride (12 mg, 0.17 mmol) in dichloromethane (1 mL) is added dropwise. After stirring at RT for 4 hours, the mixture is partitioned between 100 mL of dichloromethane and 100 mL of brine. The organic layer is separated from aqueous layer, washed with brine (100 mL×2) and dried over Na₂SO₄. The material is concentrated and purified by Pre-TLC (eluted with dichloromethane/MeOH=10/1) to afford 4 mg (5.5%) of 1-(1-acryloylpiperidin-3-yl)-5-amino-3-(4-phenoxyphenyl)-1H-pyrazole-4-carboxamide as a white solid.

The enantiomers of Formula (23) provided by the procedure above may be prepared from 5-amino-3-(phenoxyphenyl)-1H-pyrazole-4-carbonitrile and (S)-tert-butyl 3-hydroxypiperidine-1-carboxylate using a similar procedure (step 4 to 8) for Formula (24), or from (R)-tert-butyl 3-hydroxypiperidine-1-carboxylate using a similar procedure (step 4 to 8) for Formula (25). Under appropriate conditions recognized by one of ordinary skill in the art, a racemic mixture of Formula (23) may be separated by chiral HPLC, the crystallization of chiral salts, or other means described above to yield Formula (24) and Formula (25) of high enantiomeric purity.

In an embodiment, the BTK inhibitor is a compound selected from the structures disclosed in U.S. Patent Application Publication No. US 2015/0005277A1, the disclosure of which is incorporated by reference herein.

In an embodiment, the BTK inhibitor is a compound selected from the structures disclosed in U.S. Pat. No. 8,957,065, the disclosure of which is incorporated by reference herein. In an embodiment, the BTK inhibitor is HM-71224 (Hanmi Pharm. Co.), or a pharmaceutically acceptable salt, solvate, hydrate, cocrystal, or prodrug thereof. In an embodiment, the BTK inhibitor is N-(3-(2-(4-(4-methylpiperazin-1-yl)phenylamino)thieno[3,2-d]pyrimidine-4-yloxy)phenyl)acrylamide, or a pharmaceutically acceptable salt, solvate, hydrate, cocrystal, or prodrug thereof. In an embodiment, the BTK inhibitor is N-(3-((2-((2-methoxy-4-(4-methylpiperazin-1-yl)phenyl)amino)thieno[3,2-d]pyrimidin-4-yl)oxy)phenyl)acrylamide, or a pharmaceutically acceptable salt, solvate, hydrate, cocrystal, or prodrug thereof.

In an embodiment, the BTK inhibitor is 7-acryloyl-2-(4-phenoxyphenyl)-5,6,7,8-tetrahydro-4H-pyrazolo[5′,1′:2,3]imidazo[4,5-c]pyridine-3-carboxamide, or a pharmaceutically acceptable salt, solvate, hydrate, cocrystal, or prodrug thereof.

Other BTK inhibitors suitable for use in the described combination with an antifolate compound include, but are not limited to, those described in International Patent Application Publication Nos. WO 2013/010868, WO 2012/158843, WO 2012/135944, WO 2012/135937, U.S. Patent Application Publication No. 2011/0177011, and U.S. Pat. Nos. 8,501,751, 8,476,284, 8,008,309, 7,960,396, 7,825,118, 7,732,454, 7,514,444, 7,459,554, 7,405,295, and 7,393,848, the disclosures of each of which are incorporated herein by reference.

### Antifolates

The antifolate compound may be any antifolate compound known in the art. In particular, it is one of the antifolate compounds described in more detail in the following paragraphs. In preferred embodiments, the compositions described herein provide a combination of an antifolate compound with a BTK inhibitor, or methods of using a combination of an antifolate compound with a BTK inhibitor. In an embodiment, an antifolate compound inhibits thymidylate synthase (TS), dihydrofolate reductase (DHFR), glycinamide ribonucleotide formyltransferase (GARFT), and combinations thereof.

In one embodiment, the antifolate compound is (2S)-2-[(4-{[(2,4-Diaminopteridin-6-yl)methyl](methyl)amino}benzoyl)amino]pentanedioic acid (“methotrexate,” also known as amethopterin) having the structure of Formula (26):

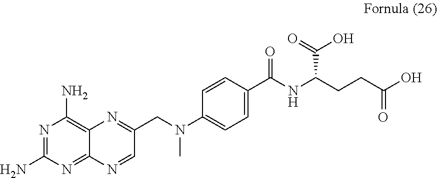 [SMILES: CN(Cc1cnc2nc(N)nc(N)c2n1)c1ccc(C(=O)N[C@@H](CCC(=O)O)C(=O)O)cc1]

or a pharmaceutically acceptable salt, solvate, hydrate, cocrystal, or prodrug thereof. The synthesis and properties of methotrexate are described in U.S. Pat. No. 2,512,572, the disclosures of which are incorporated by reference herein in its entirety. Methotrexate is commercially available from multiple suppliers under brandnames such as TREXALL, RHEUMATREX, OTREXUP, and RASUVO.

In one embodiment, the antifolate compound is (2S)-2-{[4-[2-(2-amino-4-oxo-1,7-dihydropyrrolo[2,3-d]pyrimidin-5-yl)ethyl]benzoyl]amino}pentanedioic acid (“pemetrexed”) having the structure of Formula (27):

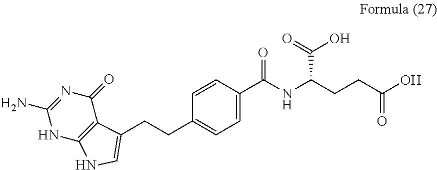 [SMILES: Nc1nc(=O)c2c(CCc3ccc(C(=O)N[C@@H](CCC(=O)O)C(=O)O)cc3)c[nH]c2[nH]1]

or a pharmaceutically acceptable salt, solvate, hydrate, cocrystal, or prodrug thereof. The synthesis and properties of pemetrexed are described in U.S. Pat. No. 5,344,932, the disclosures of which are incorporated by reference herein in its entirety. Pemetrexed is commercially available as ALIMTA (Eli Lilly & Co.).

In one embodiment, the antifolate compound is N-[(5-{methyl[(2-methyl-4-oxo-1,4-dihydroquinazolin-6-yl)methyl]amino}-2-thienyl)carbonyl]-L-glutamic acid (“raltitrexed”) having the structure of Formula (28):

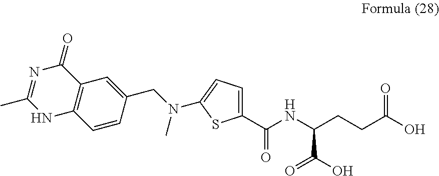 [SMILES: Cc1nc(=O)c2cc(CN(C)c3ccc(C(=O)N[C@@H](CCC(=O)O)C(=O)O)s3)ccc2[nH]1]

or a pharmaceutically acceptable salt, solvate, hydrate, cocrystal, or prodrug thereof. The synthesis and properties of raltitrexed are described in U.S. Pat. No. 4,992,550, the disclosures of which are incorporated by reference herein in its entirety. Raltitrexed is commercially available as TOMUDEX (AstraZeneca plc.).

### Pharmaceutical Compositions

In one embodiment, the invention provides a pharmaceutical composition for use in the treatment of the diseases and conditions described herein. In a preferred embodiment, the invention provides pharmaceutical compositions, including those described below, for use in the treatment of a hyperproliferative disorder. In a preferred embodiment, the invention provides pharmaceutical compositions, including those described below, for use in the treatment of cancer. In preferred embodiment, the invention provides for pharmaceutical compositions, including those described below, for use in treatment of inflammatory, immune or autoimmune disorders.

In some embodiments, the invention provides pharmaceutical compositions for treating solid tumor cancers, lymphomas and leukemia.

In some embodiments, the invention provides pharmaceutical compositions for treating arthritis.

In preferred embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor or a pharmaceutically acceptable salt, solvate, hydrate, cocrystal, or prodrug thereof, for use in the treatment of cancer. This composition is typically a pharmaceutical composition.

In preferred embodiments, the invention provides a composition comprising therapeutically effective amounts of (1) an antifolate compound or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor having the structure:

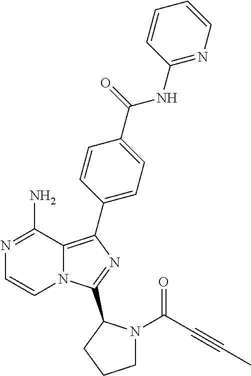 [SMILES: CC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor having the structure:

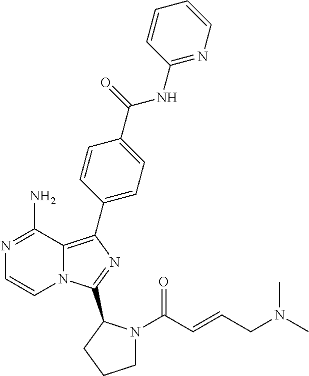 [SMILES: CN(C)C/C=C/C(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor having the structure:

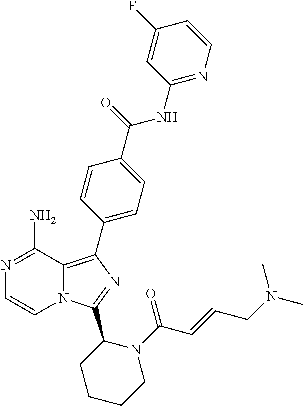 [SMILES: CN(C)C/C=C/C(=O)N1CCCC[C@H]1c1nc(-c2ccc(C(=O)Nc3cc(F)ccn3)cc2)c2c(N)nccn12]

or a pharmaceutically acceptable salt, solvate, hydrate, cocrystal, or prodrug thereof.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor having the structure:

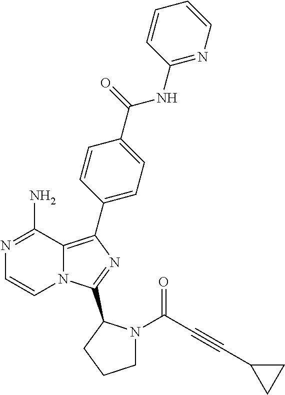 [SMILES: Nc1nccn2c([C@@H]3CCCN3C(=O)C#CC3CC3)nc(-c3ccc(C(=O)Nc4ccccn4)cc3)c12]

or a pharmaceutically acceptable salt, solvate, hydrate, cocrystal, or prodrug thereof.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor having the structure:

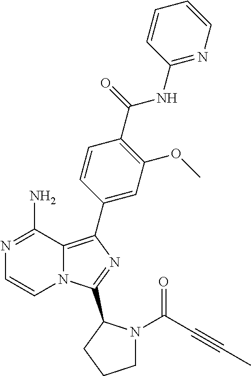 [SMILES: CC#CC(=O)N1CCC[C@H]1c1nc(-c2ccc(C(=O)Nc3ccccn3)c(OC)c2)c2c(N)nccn12]

or a pharmaceutically acceptable salt, solvate, hydrate, cocrystal, or prodrug thereof.

In some embodiments, the invention provides a composition comprising therapeutically effective amounts of (1) an antifolate compound or a pharmaceutically acceptable salt, solvate, hydrate, cocrystal, or prodrug thereof; and (2) a BTK inhibitor selected from the group consisting of:

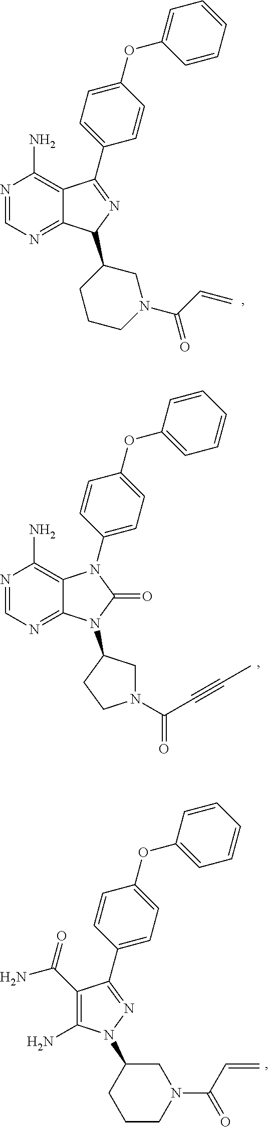 [SMILES: C=CC(=O)N1CCC[C@@H](C2N=C(c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof. This composition is typically a pharmaceutical composition.

In some embodiments, the invention provides a composition comprising therapeutically effective amounts of (1) an antifolate compound or a pharmaceutically acceptable salt, solvate, hydrate, cocrystal, or prodrug thereof; (2) a BTK inhibitor selected from the group consisting of:

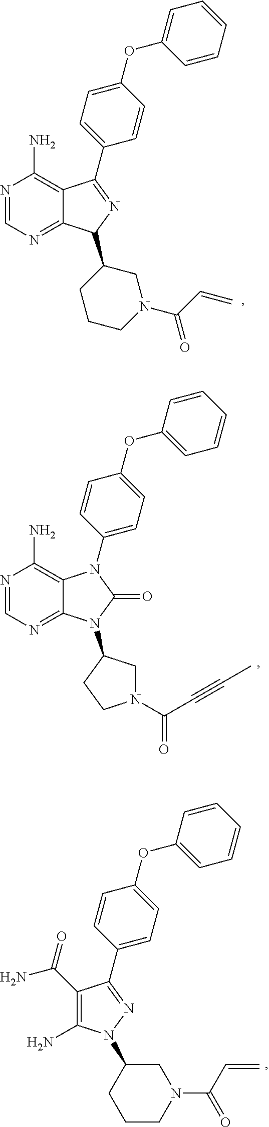 [SMILES: C=CC(=O)N1CCC[C@@H](C2N=C(c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an antifolate compound selected from the group consisting of methotrexate, pemetrexed, raltitrexed and pharmaceutically acceptable salts, solvates, hydrates, cocrystals, prodrugs, and combinations thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an antifolate compound selected from the group consisting of methotrexate, pemetrexed, raltitrexed and pharmaceutically acceptable salts, solvates, hydrates, cocrystals, prodrugs, and combinations thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an antifolate compound selected from the group consisting of methotrexate, pemetrexed, raltitrexed and pharmaceutically acceptable salts, solvates, hydrates, cocrystals, prodrugs, and combinations thereof; (2) a BTK inhibitor or a pharmaceutically acceptable salt, solvate, hydrate, cocrystal, or prodrug thereof, for use in the treatment of cancer; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

The pharmaceutical compositions are typically formulated to provide a therapeutically effective amount of a combination as described herein, i.e., a combination of an antifolate compound and a BTK inhibitor as the active ingredients, or pharmaceutically acceptable salts, prodrugs, solvates, or hydrates thereof. Where desired, the pharmaceutical compositions contain a pharmaceutically acceptable salt and/or coordination complex of one or more of the active ingredients. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of the diseases and conditions described below. In a preferred embodiment, the pharmaceutical compositions are for use in the treatment of cancer. In preferred embodiments, the pharmaceutical compositions are for use in treating solid tumor cancers, lymphomas, and leukemias. In preferred embodiments, the pharmaceutical compositions are for use in treating inflammatory, immune or autoimmune disorders.

In a preferred embodiment, the pharmaceutical compositions of the present invention are for use in the treatment of cancer. In one embodiment, the pharmaceutical compositions of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In a preferred embodiment, the pharmaceutical compositions of the present invention are for use in the treatment of an inflammatory, immune, or autoimmune disorder. In one embodiment, the pharmaceutical compositions of the present invention are for use in the treatment of an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitiligo.

The pharmaceutical compositions may be administered as a combination of an antifolate compound and a BTK inhibitor. Where desired, other active pharmaceutical ingredient(s) may be mixed into a preparation or two or more components of the combination may be formulated into separate preparations for use in combination separately or at the same time. A kit containing the components of the combination, formulated into separate preparations for said use, in also provided by the invention.

In an embodiment, the molar ratio of the antifolate compound to the BTK inhibitor in the pharmaceutical compositions is in the range from about 10:1 to about 1:20, preferably from about 2.5:1 to about 1:2.5, and more preferably about 1:1. In an embodiment, the weight ratio of the antifolate compound to the BTK inhibitor in the pharmaceutical compositions is selected from the group consisting of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, and about 1:20.

In some embodiments, the concentration of any one or two of the antifolate compounds and BTK inhibitors provided in the pharmaceutical compositions of the invention is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of any one or two of the antifolate compounds and BTK inhibitors provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of any one or two of the antifolate compounds and BTK inhibitors provided in the pharmaceutical compositions is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of any one or two of the antifolate compounds and BTK inhibitors provided in the pharmaceutical compositions is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of any one or two of the antifolate compounds and BTK inhibitors provided in the pharmaceutical compositions is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of any one or two of the antifolate compounds and BTK inhibitors provided in the pharmaceutical compositions is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the antifolates and BTK inhibitors according to the invention is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the invention provides a pharmaceutical composition for oral administration containing the combination of an antifolate compound and a BTK inhibitor, and a pharmaceutical excipient suitable for oral administration.

In preferred embodiments, the invention provides a solid pharmaceutical composition for oral administration containing: (i) an effective amount of each of an antifolate compound and a BTK inhibitor in combination and (ii) a pharmaceutical excipient suitable for oral administration. In some embodiments, the composition further contains (iii) an effective amount of a third or fourth active pharmaceutical ingredient.

In some embodiments, the pharmaceutical composition may be a liquid pharmaceutical composition suitable for oral consumption.

Pharmaceutical compositions of the invention suitable for oral administration can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the antifolate compounds and BTK inhibitors as active ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; camitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the invention provides a pharmaceutical composition for injection containing the combination of the antifolate compounds and BTK inhibitors, and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating the combination of the antifolate compounds and BTK inhibitors in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the invention provides a pharmaceutical composition for transdermal delivery containing the combination of the antifolate compounds and BTK inhibitors, and a pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of the combination of the antifolate compounds and BTK inhibitors in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of the combination of the antifolate compounds and BTK inhibitors or pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. The combination of the antifolate compounds and BTK inhibitors may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. The combination of the antifolate compounds and BTK inhibitors may additionally be administered intravascularly from a balloon used during angioplasty. Extravascular administration of the combination of the antifolate compounds and BTK inhibitors via the pericard or via advential application of formulations of the invention may also be performed to decrease restenosis.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include each of the antifolate compounds and BTK inhibitors, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, the antifolate compounds and BTK inhibitors and another active pharmaceutical ingredient are provided as separate compositions in separate containers within the kit. In selected embodiments, the antifolate compounds and BTK inhibitors and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising (1) a composition comprising a therapeutically effective amount of an antifolate compound or fragments, derivatives, conjugates, variants, biosimilars, and combinations thereof, and (2) a composition comprising a therapeutically effective amount of a BTK inhibitor or a pharmaceutically acceptable salt, solvate, hydrate, cocrystal, or prodrug thereof. These compositions are typically pharmaceutical compositions. The kit is for co-administration of the antifolate compounds and the BTK inhibitors, either simultaneously or separately.

In some embodiments, the invention provides a kit comprising (1) a composition comprising a therapeutically effective amount of an antifolate compounds or fragments, derivatives, conjugates, variants, biosimilars, and combinations thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. These compositions are typically pharmaceutical compositions. The kit is for co-administration of the antifolate compound, the BTK inhibitor, and/or the anti-CD20 antibody, either simultaneously or separately.

The kits described above are preferably for use in the treatment of the diseases and conditions described herein. In a preferred embodiment, the kits are for use in the treatment of cancer. In preferred embodiments, the kits are for use in treating solid tumor cancers, lymphomas and leukemias. In preferred embodiments, the kits are for use in treating inflammatory, immune or autoimmune disorders.

In a preferred embodiment, the kits of the present invention are for use in the treatment of cancer. In a preferred embodiment, the kits of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In a preferred embodiment, the kits of the present invention are for use in the treatment of an inflammatory, immune, or autoimmune disorder. In one embodiment, the kits of the present invention are for use in the treatment of an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitiligo.

### Dosages and Dosing Regimens

The amounts of BTK inhibitors and antifolate compounds administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of BTK inhibitors and antifolate compounds may be provided in units of mg/kg of body mass or in mg/m² of body surface area. In an embodiment, the ratio of the dose of the antifolate compound to the dose of the BTK inhibitor in mg/kg or in mg/m² is in the range from 10:1 to 1:10, preferably from 2.5:1 to 1:2.5, and more preferably about 1:1. In an embodiment, the ratio of the antifolate compound to the BTK inhibitor in mg/kg or in mg/m² is selected from the group consisting of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, and about 1:20.

In some embodiments, the combination of the antifolate compound and BTK inhibitor is administered in a single dose. Such administration may be by injection, e.g., intravenous injection, in order to introduce the antifolate compound and BTK inhibitor quickly. However, other routes, including the preferred oral route, may be used as appropriate. A single dose of the combination of the antifolate compound and BTK inhibitor may also be used for treatment of an acute condition.

In some embodiments, the combination of the antifolate compound and BTK inhibitor is administered in multiple doses. In a preferred embodiment, the combination of the antifolate compound and BTK inhibitor is administered in multiple doses. Dosing may be once, twice, three times, four times, five times, six times, or more than six times per day. Dosing may be once a month, once every two weeks, once a week, or once every other day. In other embodiments, the combination of the antifolate compound and BTK inhibitor is administered about once per day to about 6 times per day. In some embodiments, the combination of the antifolate compound and BTK inhibitor is administered once daily, while in other embodiments, the combination of the antifolate compound and BTK inhibitor is administered twice daily, and in other embodiments the combination of the antifolate compound and BTK inhibitor is administered three times daily.

Administration of the active pharmaceutical ingredients of the invention may continue as long as necessary. In selected embodiments, the combination of the antifolate compound and BTK inhibitor is administered for more than 1, 2, 3, 4, 5, 6, 7, 14, or 28 days. In some embodiments, the combination of the antifolate compound and BTK inhibitor is administered for less than 28, 14, 7, 6, 5, 4, 3, 2, or 1 day. In selected embodiments, the combination of the antifolate compound and BTK inhibitor is administered chronically on an ongoing basis—e.g., for the treatment of chronic effects. In another embodiment the administration of the combination of the antifolate compound and BTK inhibitor continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of a BTK inhibitor disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a BTK inhibitor disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of a BTK inhibitor disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a BTK inhibitor disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an antifolate compound disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an antifolate compound disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an antifolate compound disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an antifolate compound disclosed herein is about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a combination of a BTK inhibitor and an antifolate compound is administered at a dosage of 10 to 200 mg BID, including 10, 20, 30, 40, 50, 60, 70, 80, 90, 100, 150, or 200 mg BID, for the BTK inhibitor, and 10 to 200 mg BID, including 25, 50, 75, 100, 150, or 200 mg BID for the antifolate compound.

In some embodiments, a combination of a BTK inhibitor and an antifolate compound is administered at a dosage of 10 to 200 mg BID, including 10, 20, 30, 40, 50, 60, 70, 80, 90, 100, or 150 mg BID, for the BTK inhibitor, and 1 to 500 mg BID, including 1, 5, 10, 15, 25, 50, 75, 100, 150, 200, 300, 400, or 500 mg BID for the antifolate compound inhibitor.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the an antifolate compound and BTK inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Methods of Treating Solid Tumor Cancers, Hematological Malignancies, Inflammation, Immune and Autoimmune Disorders, and Other Diseases

The compositions and combinations of inhibitors described above can be used in a method for treating BTK-mediated disorders and diseases. In a preferred embodiment, they are for use in treating hyperproliferative disorders. They may also be used in treating other disorders as described herein and in the following paragraphs.

In some embodiments, the invention provides a method of treating a hyperproliferative disorder in a mammal that comprises administering to said mammal a therapeutically effective amount of an antifolate compound and a BTK inhibitor, or a pharmaceutically acceptable salt, solvate, hydrate, cocrystal, or prodrug of either or both the antifolate compound or the BTK inhibitor.

In some embodiments, the invention provides a method of treating a hyperproliferative disorder in a mammal that comprises administering to said mammal a therapeutically effective amount of a BTK inhibitor, wherein the BTK inhibitor is selected from wherein the BTK inhibitor is selected from the group consisting of Formula (2), Formula (3), Formula (4), Formula (5), Formula (6), and Formula (7), or a pharmaceutically acceptable salt, solvate, hydrate, cocrystal, or prodrug thereof. In some embodiments, the invention provides a method of treating a hyperproliferative disorder in a mammal that comprises administering to said mammal a therapeutically effective amount of an antifolate compound and a BTK inhibitor, where the BTK inhibitor is selected from the group consisting of wherein the BTK inhibitor is selected from the group consisting of Formula (2), Formula (3), Formula (4), Formula (5), Formula (6), and Formula (7), or a pharmaceutically acceptable salt, solvate, hydrate, cocrystal, or prodrug of either or both the antifolate compound or the BTK inhibitor.

In some embodiments, the hyperproliferative disorder is a solid tumor cancer selected from the group consisting of bladder cancer, squamous cell carcinoma, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancers such as cervical carcinoma (human papillomavirus), B-cell lymphoproliferative disease, nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), glioblastoma, esophogeal tumors, head and neck tumor, metastatic colon cancer, head and neck squamous cell carcinoma, ovary tumor, pancreas tumor, renal cell carcinoma, hematological neoplasms, small-cell lung cancer, non-small-cell lung cancer, stage IV melanoma, and glioma.

In some embodiments, the hyperproliferative disorder is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenstrm's macroglobulinemia (WM), Burkitt's lymphoma, multiple myeloma, myelodysplatic syndromes, or myelofibrosis. In an embodiment, the invention relates to a method of treating a cancer in a mammal, wherein the cancer is chronic myelocytic leukemia, acute myeloid leukemia, DLBCL (including activated B-cell (ABC) and germinal center B-cell (GCB) subtypes), follicle center lymphoma, Hodgkin's disease, multiple myeloma, indolent non-Hodgkin's lymphoma, and mature B-cell ALL.

In some embodiments, the hyperproliferative disorder is a subtype of CLL. A number of subtypes of CLL have been characterized. CLL is often classified for immunoglobulin heavy-chain variable-region (IgV_{H}) mutational status in leukemic cells. Damle, et al., Blood 1999, 94, 1840-47; Hamblin, et al., Blood 1999, 94, 1848-54. Patients with IgV_{H} mutations generally survive longer than patients without IgV_{H} mutations. ZAP70 expression (positive or negative) is also used to characterize CLL. Rassenti, et al., N. Engl. J. Med. 2004, 351, 893-901. The methylation of ZAP-70 at CpG3 is also used to characterize CLL, for example by pyrosequencing. Claus, et al., J. Clin. Oncol. 2012, 30, 2483-91; Woyach, et al., Blood 2014, 123, 1810-17. CLL is also classified by stage of disease under the Binet or Rai criteria. Binet, et al., Cancer 1977, 40, 855-64; Rai, Han, Hematol. Oncol. Clin. North Am. 1990, 4, 447-56. Other common mutations, such as 11q deletion, 13q deletion, and 17p deletion can be assessed using well-known techniques such as fluorescence in situ hybridization (FISH). In an embodiment, the invention relates to a method of treating a CLL in a human, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In some embodiments, the hyperproliferative disorder is a CLL wherein the CLL has undergone a Richter's transformation. Methods of assessing Richter's transformation, which is also known as Richter's syndrome, are described in Jain and O'Brien, Oncology, 2012, 26, 1146-52. Richter's transformation is a subtype of CLL that is observed in 5-10% of patients. It involves the development of aggressive lymphoma from CLL and has a generally poor prognosis.

In some embodiments, the hyperproliferative disorder is a CLL or SLL in a patient, wherein the patient is sensitive to lymphocytosis. In an embodiment, the invention relates to a method of treating CLL or SLL in a patient, wherein the patient exhibits lymphocytosis caused by a disorder selected from the group consisting of a viral infection, a bacterial infection, a protozoal infection, or a post-splenectomy state. In an embodiment, the viral infection in any of the foregoing embodiments is selected from the group consisting of infectious mononucleosis, hepatitis, and cytomegalovirus. In an embodiment, the bacterial infection in any of the foregoing embodiments is selected from the group consisting of pertussis, tuberculosis, and brucellosis.

In some embodiments, the hyperproliferative disorder is selected from the group consisting of myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis.

In some embodiments, the hyperproliferative disorder is an inflammatory, immune, or autoimmune disorder. In some embodiments, the hyperproliferative disorder is selected from the group consisting of tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitiligo.

In some embodiments, the hyperproliferative disorder is a disease related to vasculogenesis or angiogenesis in a mammal which can manifest as tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In some embodiments, provided herein is a method of treating, preventing and/or managing asthma. As used herein, “asthma” encompasses airway constriction regardless of the cause. Common triggers of asthma include, but are not limited to, exposure to an environmental stimulants (e.g., allergens), cold air, warm air, perfume, moist air, exercise or exertion, and emotional stress. Also provided herein is a method of treating, preventing and/or managing one or more symptoms associated with asthma. Examples of the symptoms include, but are not limited to, severe coughing, airway constriction and mucus production.

Efficacy of the methods, compounds, and combinations of compounds described herein in treating, preventing and/or managing the indicated diseases or disorders can be tested using various animal models known in the art. Efficacy in treating, preventing and/or managing asthma can be assessed using the ova induced asthma model described, for example, in Lee, et al., J. Allergy Clin. Immunol. 2006, 118, 403-9. Efficacy in treating, preventing and/or managing arthritis (e.g., rheumatoid or psoriatic arthritis) can be assessed using the autoimmune animal models described in, for example, Williams, et al., Chem. Biol. 2010, 17, 123-34, WO 2009/088986, WO 2009/088880, and WO 2011/008302. Efficacy in treating, preventing and/or managing psoriasis can be assessed using transgenic or knockout mouse model with targeted mutations in epidermis, vasculature or immune cells, mouse model resulting from spontaneous mutations, and immuno-deficient mouse model with xenotransplantation of human skin or immune cells, all of which are described, for example, in Boehncke, et al., Clinics in Dermatology, 2007, 25, 596-605. Efficacy in treating, preventing and/or managing fibrosis or fibrotic conditions can be assessed using the unilateral ureteral obstruction model of renal fibrosis, which is described, for example, in Chevalier, et al., Kidney International 2009, 75, 1145-1152; the bleomycin induced model of pulmonary fibrosis described in, for example, Moore, et al., Am. J. Physiol. Lung. Cell. Mol. Physiol. 2008, 294, L152-L160; a variety of liver/biliary fibrosis models described in, for example, Chuang, et al., Clin. Liver Dis. 2008, 12, 333-347 and Omenetti, et al., Laboratory Investigation, 2007, 87, 499-514 (biliary duct-ligated model); or any of a number of myelofibrosis mouse models such as described in Varicchio, et al., Expert Rev. Hematol. 2009, 2(3), 315-334. Efficacy in treating, preventing and/or managing scleroderma can be assessed using a mouse model induced by repeated local injections of bleomycin described, for example, in Yamamoto, et al., J. Invest. Dermatol. 1999, 112, 456-462. Efficacy in treating, preventing and/or managing dermatomyositis can be assessed using a myositis mouse model induced by immunization with rabbit myosin as described, for example, in Phyanagi, et al., Arthritis & Rheumatism, 2009, 60(10), 3118-3127. Efficacy in treating, preventing and/or managing lupus can be assessed using various animal models described, for example, in Ghoreishi, et al., Lupus, 2009, 19, 1029-1035; Ohl, et al., J Biomed. Biotechnol., 2011, Article ID 432595; Xia, et al., Rheumatology, 2011, 50, 2187-2196; Pau, et al., PLoS ONE, 2012, 7(5), e36761; Mustafa, et al., Toxicology, 2011, 290, 156-168; Ichikawa, et al., Arthritis & Rheumatism, 2012, 62(2), 493-503; Rankin, et al., J. Immunology, 2012, 188, 1656-1667. Efficacy in treating, preventing and/or managing Sjögren's syndrome can be assessed using various mouse models described, for example, in Chiorini, et al., J. Autoimmunity, 2009, 33, 190-196. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980.

In selected embodiments, the invention provides a method of treating a solid tumor cancer with a composition including a combination of an antifolate compound and a BTK inhibitor, wherein the dose is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In selected embodiments, the invention provides a method of treating pancreatic cancer, breast cancer, ovarian cancer, melanoma, lung cancer, squamous cell carcinoma including head and neck cancer, and colorectal cancer using a combination of a BTK inhibitor and an antifolate compound, wherein the dose is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts.

In some embodiments, the invention provides pharmaceutical compositions of a combination of a BTK inhibitor and an antifolate compound for the treatment of hyperproliferative disorders as described herein. In some embodiments, the invention provides pharmaceutical compositions of a combination of a BTK inhibitor and an antifolate compound for the treatment of disorders such as myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis, wherein the BTK inhibitor is selected from the group consisting of wherein the BTK inhibitor is selected from the group consisting of Formula (1), Formula (2), Formula (3), Formula (4), Formula (5), Formula (6), and Formula (7). The invention further provides a composition as described herein for the prevention of blastocyte implantation in a mammal.

### Methods of Treating Patients Intolerant to Bleeding Events

In selected embodiments, the invention provides a method of treating a disease in a human sensitive to or intolerant to bleeding events, comprising the step of administering a therapeutically effective amount of a BTK inhibitor, or a pharmaceutically-acceptable salt, cocrystal, hydrate, solvate, or prodrug thereof, and an antifolate compound, or a pharmaceutically-acceptable salt, cocrystal, hydrate, solvate, or prodrug thereof. In a preferred embodiment, the invention provides a method of treating a hyperproliferative disorder in a human sensitive to or intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof. In a preferred embodiment, the invention provides a method of treating a hyperproliferative disorder in a human sensitive to or intolerant to bleeding events, comprising the step of administering a therapeutically effective amount of a BTK inhibitor and an antifolate compound, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof, and wherein the antifolate compound is selected from the group consisting of methotrexate, pemetrexed, raltitrexed and pharmaceutically acceptable salts, solvates, hydrates, cocrystals, prodrugs, and combinations thereof. In some embodiments, the invention provides a method of treating a disease in a human sensitive to or intolerant to ibrutinib.

In selected embodiments, the invention provides a method of treating a disease in a human sensitive to or intolerant to bleeding events, comprising the step of administering a therapeutically effective amount of a BTK inhibitor, or a pharmaceutically-acceptable salt, cocrystal, hydrate, solvate, or prodrug thereof, and an antifolate compound or a pharmaceutically-acceptable salt, cocrystal, hydrate, solvate, or prodrug thereof. In a preferred embodiment, the invention provides a method of treating a cancer in a human sensitive to or intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof. In a preferred embodiment, the invention provides a method of treating a cancer in a human sensitive to or intolerant to bleeding events, comprising the step of administering a therapeutically effective amount of a BTK inhibitor and an antifolate compound, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof, and wherein the antifolate compound is selected from the group consisting of methotrexate, pemetrexed, raltitrexed and pharmaceutically acceptable salts, solvates, hydrates, cocrystals, prodrugs, and combinations thereof.

In an embodiment, the invention provides a method of treating a cancer in a human intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), or a pharmaceutically-acceptable salt, cocrystal, hydrate, solvate, or prodrug thereof, and an antifolate compound, or fragments, derivatives, conjugates, variants, biosimilars, and combinations thereof, further comprising the step of administering a therapeutically effective amount of an anticoagulant or antiplatelet active pharmaceutical ingredient.

In selected embodiments, the invention provides a method of treating a cancer in a human intolerant to bleeding events, comprising the step of administering a therapeutically effective amount of a BTK inhibitor, wherein the BTK inhibitor is preferably is selected from the group consisting of Formula (1), Formula (2), Formula (3), Formula (4), Formula (5), Formula (6), and Formula (7), and wherein the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, head, neck, renal cancer, kidney cancer, liver cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the invention provides a method of treating a cancer in a human intolerant to platelet-mediated thrombosis comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), or a pharmaceutically-acceptable salt, cocrystal, hydrate, solvate, or prodrug thereof, and an antifolate compound selected from the group consisting of methotrexate, pemetrexed, raltitrexed and pharmaceutically acceptable salts, solvates, hydrates, cocrystals, prodrugs, and combinations thereof.

In some embodiments, the BTK inhibitor and the anticoagulant or the antiplatelet active pharmaceutical ingredient are administered sequentially. In some embodiments, the BTK inhibitor and the anticoagulant or the antiplatelet active pharmaceutical ingredient are administered concomitantly. In selected embodiments, the BTK inhibitor is administered before the anticoagulant or the antiplatelet active pharmaceutical ingredient. In selected embodiments, the BTK inhibitor is administered after the anticoagulant or the antiplatelet active pharmaceutical ingredient. In selected embodiments, an antifolate compound is co-administered with the BTK inhibitor and the anticoagulant or the antiplatelet active pharmaceutical ingredient at the same time or at different times.

Selected anti-platelet and anticoagulant active pharmaceutical ingredients for use in the methods of the present invention include, but are not limited to, cyclooxygenase inhibitors (e.g., aspirin), adenosine diphosphate (ADP) receptor inhibitors (e.g., clopidogrel and ticlopidine), phosphodiesterase inhibitors (e.g., cilostazol), glycoprotein IIb/IIIa inhibitors (e.g., abciximab, eptifibatide, and tirofiban), and adenosine reuptake inhibitors (e.g., dipyridamole). In other embodiments, examples of anti-platelet active pharmaceutical ingredients for use in the methods of the present invention include anagrelide, aspirin/extended-release dipyridamole, cilostazol, clopidogrel, dipyridamole, prasugrel, ticagrelor, ticlopidine, vorapaxar, tirofiban HCl, eptifibatide, abciximab, argatroban, bivalirudin, daltcparin, desirudin, enoxaparin, fondaparinux, heparin, lepirudin, apixaban, dabigatran etexilate mesylate, rivaroxaban, and warfarin.

In an embodiment, the invention provides a method of treating a cancer, comprising the step of orally administering, to a human in need thereof, a Bruton's tyrosine kinase (BTK) inhibitor, wherein the BTK inhibitor is (S)-4-(8-amino-3-(1-(but-2-ynoyl)pyrrolidin-2-yl)imidazo[1,5-a]pyrazin-1-yl)-N-(pyridin-2-yl)benzamide or a pharmaceutically acceptable salt, solvate, hydrate, cocrystal, or prodrug thereof, and an antifolate compound, or pharmaceutically acceptable salt, solvate, hydrate, cocrystal, or prodrug thereof, further comprising the step of administering a therapeutically effective amount of an anticoagulant or antiplatelet active pharmaceutical ingredient, wherein the anticoagulant or antiplatelet active pharmaceutical ingredient is selected from the group consisting of acenocoumarol, anagrelide, anagrelide hydrochloride, abciximab, aloxiprin, antithrombin, apixaban, argatroban, aspirin, aspirin with extended-release dipyridamole, beraprost, betrixaban, bivalirudin, carbasalate calcium, cilostazol, clopidogrel, clopidogrel bisulfate, cloricromen, dabigatran etexilate, darexaban, dalteparin, dalteparin sodium, defibrotide, dicumarol, diphenadione, dipyridamole, ditazole, desirudin, edoxaban, enoxaparin, enoxaparin sodium, eptifibatide, fondaparinux, fondaparinux sodium, heparin, heparin sodium, heparin calcium, idraparinux, idraparinux sodium, iloprost, indobufen, lepirudin, low molecular weight heparin, melagatran, nadroparin, otamixaban, parnaparin, phenindione, phenprocoumon, prasugrel, picotamide, prostacyclin, ramatroban, reviparin, rivaroxaban, sulodexide, terutroban, terutroban sodium, ticagrelor, ticlopidine, ticlopidine hydrochloride, tinzaparin, tinzaparin sodium, tirofiban, tirofiban hydrochloride, treprostinil, treprostinil sodium, triflusal, vorapaxar, warfarin, warfarin sodium, ximelagatran, salts thereof, solvates thereof, hydrates thereof, prodrugs thereof, and combinations thereof.

In selected embodiments, the invention provides a method of treating a hyperproliferative disorder in a human sensitive to or intolerant to bleeding events, comprising the step of administering a therapeutically effective amount of a BTK inhibitor, or a pharmaceutically-acceptable salt, cocrystal, hydrate, solvate, or prodrug thereof, and an antifolate compound or a pharmaceutically-acceptable salt, cocrystal, hydrate, solvate, or prodrug thereof. In a preferred embodiment, the invention provides a method of treating an inflammatory, immune, or autoimmune disorder in a human sensitive to or intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof. In a preferred embodiment, the invention provides a method of treating an inflammatory, immune, or autoimmune disorder in a human sensitive to or intolerant to bleeding events, comprising the step of administering a therapeutically effective amount of a BTK inhibitor and an antifolate compound, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof, and wherein the antifolate compound is selected from the group consisting of methotrexate, pemetrexed, raltitrexed and pharmaceutically acceptable salts, solvates, hydrates, cocrystals, prodrugs, and combinations thereof.

In some embodiments, the inflammatory, immune, or autoimmune disorder is selected from the group consisting of tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitiligo.

### Combinations of BTK Inhibitors, Antifolate Compounds, and Anti-CD20 Antibodies

The BTK inhibitors of the present invention and combinations of the BTK inhibitors with antifolate compounds may also be safely co-administered with immunotherapeutic antibodies such as the anti-CD20 antibodies rituximab, obinutuzumab, ofatumumab, veltuzumab, tositumomab, and ibritumomab, and or antigen-binding fragments, derivatives, conjugates, variants, and radioisotope-labeled complexes thereof, which may be given alone or with conventional chemotherapeutic active pharmaceutical ingredients such as those described herein. In an embodiment, the foregoing combinations exhibit synergistic effects that may result in greater efficacy, less side effects, the use of less active pharmaceutical ingredient to achieve a given clinical result, or other synergistic effects.

In an embodiment, the invention provides a method of treating a hyperproliferative disorder, including an inflammatory, immune or autoimmune disorder, a hematological malignancy or a solid tumor cancer, in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and further comprising the step of administering an anti-CD20 antibody, wherein the anti-CD20 antibody is a monoclonal antibody or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and further comprising the step of administering an anti-CD20 antibody, wherein the anti-CD20 antibody is an anti-CD20 monoclonal antibody or an antigen-binding fragment, derivative, conjugate, variant, or radioisotope-labeled complex thereof, and wherein the anti-CD20 antibody specifically binds to human CD20 with a K_{D} selected from the group consisting of 1×10⁻⁷ M or less, 5×10⁻⁸ M or less, 1×10⁻⁸ M or less, and 5×10⁻⁹ M or less. Anti-CD20 monoclonal antibodies are classified as Type I or Type II, as described in Klein, et al., mAbs 2013, 5, 22-33. Type I anti-CD20 monoclonal antibodies are characterized by binding to the Class I epitope, localization of CD20 to lipid rafts, high complement-dependent cytotoxicity, full binding capacity, weak homotypic aggregation, and moderate cell death induction. Type II anti-CD20 monoclonal antibodies are characterized by binding to the Class I epitope, a lack of localization of CD20 to lipid rafts, low complement-dependent cytotoxicity, half binding capacity, homotypic aggregation, and strong cell death induction. Both Type I and Type II anti-CD20 monoclonal antibodies exhibit antibody-dependent cytotoxiticy (ADCC) and are thus useful with BTK inhibitors described herein. Type I anti-CD20 monoclonal antibodies include but are not limited to rituximab, ocrelizumab, and ofatumumab. Type II anti-CD20 monoclonal antibodies include but are not limited to obinutuzumab and tositumomab. In an embodiment, the foregoing methods exhibit synergistic effects that may result in greater efficacy, less side effects, the use of less active pharmaceutical ingredient to achieve a given clinical result, or other synergistic effects.

In an embodiment, the invention provides a method of treating a hyperproliferative disorder, including an inflammatory, immune or autoimmune disorder, a hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and an antifolate compound or fragments, derivatives, conjugates, variants, biosimilars, and combinations thereof, and further comprising the step of administering an anti-CD20 antibody, wherein the anti-CD20 antibody is a monoclonal antibody or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, an antifolate compound selected from the group consisting of methotrexate, pemetrexed, raltitrexed and pharmaceutically acceptable salts, solvates, hydrates, cocrystals, prodrugs, and combinations thereof, and further comprising the step of administering an anti-CD20 antibody, wherein the anti-CD20 antibody is an anti-CD20 monoclonal antibody or an antigen-binding fragment, derivative, conjugate, variant, or radioisotope-labeled complex thereof, and wherein the anti-CD20 antibody specifically binds to human CD20 with a K_{D} selected from the group consisting of 1×10⁻⁷ M or less, 5×10⁻⁸ M or less, 1×10⁻⁸ M or less, and 5×10⁻⁹ M or less.

In an embodiment, the invention provides a method of treating a hyperproliferative disorder, including an inflammatory, immune or autoimmune disorder, a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering an Type I anti-CD20 antibody, or an antigen-binding fragment, derivative, conjugate, variant, or radioisotope-labeled complex thereof. In an embodiment, the invention provides a method of treating a hyperproliferative disorder, including an inflammatory, immune or autoimmune disorder, hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and further comprising the step of administering an Type II anti-CD20 antibody, or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and an antifolate compound or fragments, derivatives, conjugates, variants, biosimilars, and combinations thereof, and further comprising the step of administering an Type I anti-CD20 antibody, or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), Formula (7), Formula (10), and Formula (21), and a pharmaceutically acceptable salt or ester, prodrug, cocrystal, solvate or hydrate thereof, and an antifolate compound or fragments, derivatives, conjugates, variants, biosimilars, and combinations thereof, and further comprising the step of administering an Type II anti-CD20 antibody, or an antigen-binding fragment, derivative, conjugate, variant, or radioisotope-labeled complex thereof.

In selected embodiments, the BTK inhibitors of the present invention and combinations of the BTK inhibitors with antifolate compounds, and the anti-CD20 monoclonal antibody are administered sequentially. In selected embodiments, the BTK inhibitors of the present invention and combinations of the BTK inhibitors with antifolate compounds, and the anti-CD20 monoclonal antibody are administered concomitantly. In selected embodiments, the BTK inhibitors of the present invention and combinations of the BTK inhibitors with antifolate compounds are administered before the anti-CD20 monoclonal antibody. In selected embodiments, the BTK inhibitors of the present invention and combinations of the BTK inhibitors with antifolate compounds are administered after the anti-CD20 monoclonal antibody. In selected embodiments, the BTK inhibitors of the present invention and combinations of the BTK inhibitors with antifolate compounds and the anti-CD20 monoclonal antibody are administered over the same time period, and the BTK inhibitor administration continues after the anti-CD20 monoclonal antibody administration is completed.

In an embodiment, the anti-CD20 monoclonal antibody is rituximab, or an antigen-binding fragment, derivative, conjugate, variant, or radioisotope-labeled complex thereof. Rituximab is a chimeric murine-human monoclonal antibody directed against CD20, and its structure comprises an IgG1 kappa immunoglobulin containing murine light- and heavy-chain variable region sequences and human constant region sequences. Rituximab is composed of two heavy chains of 451 amino acids and two light chains of 213 amino acids. The amino acid sequence for the heavy chains of rituximab is set forth in SEQ ID NO: 1. The amino acid sequence for the light chains of rituximab is set forth in SEQ ID NO:2. Rituximab is commercially available, and its properties and use in cancer and other diseases is described in more detail in Rastetter, et al., Ann. Rev. Med. 2004, 55, 477-503, and in Plosker and Figgett, Drugs, 2003, 63, 803-43. In an embodiment, the anti-CD20 monoclonal antibody is an anti-CD20 biosimilar monoclonal antibody approved by drug regulatory authorities with reference to rituximab. In an embodiment, the anti-CD20 monoclonal antibody has a heavy chain sequence identity of greater than 90% to SEQ ID NO: 1. In an embodiment, the anti-CD20 monoclonal antibody has a light chain sequence identity of greater than 90% to SEQ ID NO:2. In an embodiment, the anti-CD20 monoclonal antibody has a heavy chain sequence identity of greater than 95% to SEQ ID NO:1. In an embodiment, the anti-CD20 monoclonal antibody has a light chain sequence identity of greater than 95% to SEQ ID NO:2. In an embodiment, the anti-CD20 monoclonal antibody has a heavy chain sequence identity of greater than 98% to SEQ ID NO: 1. In an embodiment, the anti-CD20 monoclonal antibody has a light chain sequence identity of greater than 98% to SEQ ID NO:2. In an embodiment, the anti-CD20 monoclonal antibody has a heavy chain sequence identity of greater than 99% to SEQ ID NO:1. In an embodiment, the anti-CD20 monoclonal antibody has a light chain sequence identity of greater than 99% to SEQ ID NO:2.

In an embodiment, the anti-CD20 monoclonal antibody is obinutuzumab, or an antigen-binding fragment, derivative, conjugate, variant, or radioisotope-labeled complex thereof. Obinutuzumab is also known as afutuzumab or GA-101. Obinutuzumab is a humanized monoclonal antibody directed against CD20. The amino acid sequence for the heavy chains of obinutuzumab is set forth in SEQ ID NO:3. The amino acid sequence for the light chains of obinutuzumab is set forth in SEQ ID NO:4. Obinutuzumab is commercially available, and its properties and use in cancer and other diseases is described in more detail in Robak, Curr. Opin. Investig. Drugs 2009, 10, 588-96. In an embodiment, the anti-CD20 monoclonal antibody is an anti-CD20 biosimilar monoclonal antibody approved by drug regulatory authorities with reference to obinutuzumab. In an embodiment, the anti-CD20 monoclonal antibody has a heavy chain sequence identity of greater than 90% to SEQ ID NO:3. In an embodiment, the anti-CD20 monoclonal antibody has a light chain sequence identity of greater than 90% to SEQ ID NO:4. In an embodiment, the anti-CD20 monoclonal antibody has a heavy chain sequence identity of greater than 95% to SEQ ID NO:3. In an embodiment, the anti-CD20 monoclonal antibody has a light chain sequence identity of greater than 95% to SEQ ID NO:4. In an embodiment, the anti-CD20 monoclonal antibody has a heavy chain sequence identity of greater than 98% to SEQ ID NO:3. In an embodiment, the anti-CD20 monoclonal antibody has a light chain sequence identity of greater than 98% to SEQ ID NO:4. In an embodiment, the anti-CD20 monoclonal antibody has a heavy chain sequence identity of greater than 99% to SEQ ID NO:3. In an embodiment, the anti-CD20 monoclonal antibody has a light chain sequence identity of greater than 99% to SEQ ID NO:4. In an embodiment, the anti-CD20 monoclonal antibody obinutuzumab is an immunoglobulin G1, anti-(human B-lymphocyte antigen CD20 (membrane-spanning 4-domains subfamily A member 1, B-lymphocyte surface antigen B1, Leu-16 or Bp35)), humanized mouse monoclonal obinutuzumab des-CH3107-K-γ1 heavy chain (222-219′)-disulfide with humanized mouse monoclonal obinutuzumab κ light chain dimer (228-228″:231-231″)-bisdisulfide antibody.

In an embodiment, the anti-CD20 monoclonal antibody is ofatumumab, or an antigen-binding fragment, derivative, conjugate, variant, or radioisotope-labeled complex thereof. Ofatumumab is described in Cheson, J. Clin. Oncol. 2010, 28, 3525-30. The crystal structure of the Fab fragment of ofatumumab has been reported in Protein Data Bank reference 3GIZ and in Du, et al., Mol. Immunol. 2009, 46, 2419-2423. Ofatumumab is commercially available, and its preparation, properties, and use in cancer and other diseases are described in more detail in U.S. Pat. No. 8,529,202 B2, the disclosure of which is incorporated herein by reference. In an embodiment, the anti-CD20 monoclonal antibody is an anti-CD20 biosimilar monoclonal antibody approved by drug regulatory authorities with reference to ofatumumab. In an embodiment, the anti-CD20 monoclonal antibody has a variable heavy chain sequence identity of greater than 90% to SEQ ID NO:5. In an embodiment, the anti-CD20 monoclonal antibody has a variable light chain sequence identity of greater than 90% to SEQ ID NO:6. In an embodiment, the anti-CD20 monoclonal antibody has a variable heavy chain sequence identity of greater than 95% to SEQ ID NO:5. In an embodiment, the anti-CD20 monoclonal antibody has a variable light chain sequence identity of greater than 95% to SEQ ID NO:6. In an embodiment, the anti-CD20 monoclonal antibody has a variable heavy chain sequence identity of greater than 98% to SEQ ID NO:5. In an embodiment, the anti-CD20 monoclonal antibody has a variable light chain sequence identity of greater than 98% to SEQ ID NO:6. In an embodiment, the anti-CD20 monoclonal antibody has a variable heavy chain sequence identity of greater than 99% to SEQ ID NO:5. In an embodiment, the anti-CD20 monoclonal antibody has a variable light chain sequence identity of greater than 99% to SEQ ID NO:6. In an embodiment, the anti-CD20 monoclonal antibody has a Fab fragment heavy chain sequence identity of greater than 90% to SEQ ID NO:7. In an embodiment, the anti-CD20 monoclonal antibody has a Fab fragment light chain sequence identity of greater than 90% to SEQ ID NO:8. In an embodiment, the anti-CD20 monoclonal antibody has a Fab fragment heavy chain sequence identity of greater than 95% to SEQ ID NO:7. In an embodiment, the anti-CD20 monoclonal antibody has a Fab fragment light chain sequence identity of greater than 95% to SEQ ID NO:8. In an embodiment, the anti-CD20 monoclonal antibody has a Fab fragment heavy chain sequence identity of greater than 98% to SEQ ID NO:7. In an embodiment, the anti-CD20 monoclonal antibody has a Fab fragment light chain sequence identity of greater than 98% to SEQ ID NO:8. In an embodiment, the anti-CD20 monoclonal antibody has a Fab fragment heavy chain sequence identity of greater than 99% to SEQ ID NO:7. In an embodiment, the anti-CD20 monoclonal antibody has a Fab fragment light chain sequence identity of greater than 99% to SEQ ID NO:8. In an embodiment, the anti-CD20 monoclonal antibody ofatumumab is an immunoglobulin G1, anti-(human B-lymphocyte antigen CD20 (membrane-spanning 4-domains subfamily A member 1, B-lymphocyte surface antigen B1, Leu-16 or Bp35)); human monoclonal ofatumumab-CD20 yl heavy chain (225-214′)-disulfide with human monoclonal ofatumumab-CD20 K light chain, dimer (231-231″:234-234″)-bisdisulfide antibody.

In an embodiment, the anti-CD20 monoclonal antibody is veltuzumab, or an antigen-binding fragment, derivative, conjugate, variant, or radioisotope-labeled complex thereof. Veltuzumab is also known as hA20. Veltuzumab is described in Goldenberg, et al., Leuk. Lymphoma 2010, 51, 747-55. In an embodiment, the anti-CD20 monoclonal antibody is an anti-CD20 biosimilar monoclonal antibody approved by drug regulatory authorities with reference to veltuzumab. In an embodiment, the anti-CD20 monoclonal antibody has a heavy chain sequence identity of greater than 90% to SEQ ID NO:9. In an embodiment, the anti-CD20 monoclonal antibody has a light chain sequence identity of greater than 90% to SEQ ID NO: 10. In an embodiment, the anti-CD20 monoclonal antibody has a heavy chain sequence identity of greater than 95% to SEQ ID NO:9. In an embodiment, the anti-CD20 monoclonal antibody has a light chain sequence identity of greater than 95% to SEQ ID NO:10. In an embodiment, the anti-CD20 monoclonal antibody has a heavy chain sequence identity of greater than 98% to SEQ ID NO:9. In an embodiment, the anti-CD20 monoclonal antibody has a light chain sequence identity of greater than 98% to SEQ ID NO:10. In an embodiment, the anti-CD20 monoclonal antibody has a heavy chain sequence identity of greater than 99% to SEQ ID NO:9. In an embodiment, the anti-CD20 monoclonal antibody has a light chain sequence identity of greater than 99% to SEQ ID NO:10. In an embodiment, the anti-CD20 monoclonal antibody ofatumumab is an immunoglobulin G1, anti-(human B-lymphocyte antigen CD20 (membrane-spanning 4-domains subfamily A member 1, Leu-16, Bp35)); [218-arginine,360-glutamic acid,362-methionine]humanized mouse monoclonal hA20 γ1 heavy chain (224-213′)-disulfide with humanized mouse monoclonal hA20 κ light chain (230-230″:233-233″)-bisdisulfide dimer

In an embodiment, the anti-CD20 monoclonal antibody is tositumomab, or an antigen-binding fragment, derivative, conjugate, variant, or radioisotope-labeled complex thereof. In an embodiment, the anti-CD20 monoclonal antibody is ¹³¹I-labeled tositumomab. In an embodiment, the anti-CD20 monoclonal antibody is an anti-CD20 biosimilar monoclonal antibody approved by drug regulatory authorities with reference to tositumomab. In an embodiment, the anti-CD20 monoclonal antibody has a heavy chain sequence identity of greater than 90% to SEQ ID NO:11. In an embodiment, the anti-CD20 monoclonal antibody has a light chain sequence identity of greater than 90% to SEQ ID NO: 12. In an embodiment, the anti-CD20 monoclonal antibody has a heavy chain sequence identity of greater than 95% to SEQ ID NO: 11. In an embodiment, the anti-CD20 monoclonal antibody has a light chain sequence identity of greater than 95% to SEQ ID NO:12. In an embodiment, the anti-CD20 monoclonal antibody has a heavy chain sequence identity of greater than 98% to SEQ ID NO: 11. In an embodiment, the anti-CD20 monoclonal antibody has a light chain sequence identity of greater than 98% to SEQ ID NO:12. In an embodiment, the anti-CD20 monoclonal antibody has a heavy chain sequence identity of greater than 99% to SEQ ID NO:11. In an embodiment, the anti-CD20 monoclonal antibody has a light chain sequence identity of greater than 99% to SEQ ID NO:12.

In an embodiment, the anti-CD20 monoclonal antibody is ibritumomab, or an antigen-binding fragment, derivative, conjugate, variant, or radioisotope-labeled complex thereof. The active form of ibritumomab used in therapy is ibritumomab tiuxetan. When used with ibritumomab, the chelator tiuxctan (diethylene triamine pentaacetic acid) is complexed with a radioactive isotope such as ⁹⁰Y or ¹¹¹In. In an embodiment, the anti-CD20 monoclonal antibody is ibritumomab tiuxetan, or radioisotope-labeled complex thereof. In an embodiment, the anti-CD20 monoclonal antibody is an anti-CD20 biosimilar monoclonal antibody approved by drug regulatory authorities with reference to tositumomab. In an embodiment, the anti-CD20 monoclonal antibody has a heavy chain sequence identity of greater than 90% to SEQ ID NO: 13. In an embodiment, the anti-CD20 monoclonal antibody has a light chain sequence identity of greater than 90% to SEQ ID NO: 14. In an embodiment, the anti-CD20 monoclonal antibody has a heavy chain sequence identity of greater than 95% to SEQ ID NO:13. In an embodiment, the anti-CD20 monoclonal antibody has a light chain sequence identity of greater than 95% to SEQ ID NO:14. In an embodiment, the anti-CD20 monoclonal antibody has a heavy chain sequence identity of greater than 98% to SEQ ID NO:13. In an embodiment, the anti-CD20 monoclonal antibody has a light chain sequence identity of greater than 98% to SEQ ID NO: 14. In an embodiment, the anti-CD20 monoclonal antibody has a heavy chain sequence identity of greater than 99% to SEQ ID NO: 13. In an embodiment, the anti-CD20 monoclonal antibody has a light chain sequence identity of greater than 99% to SEQ ID NO:14.

In an embodiment, an anti-CD20 antibody selected from the group consisting of obinutuzumab, ofatumumab, veltuzumab, tositumomab, and ibritumomab, and or antigen-binding fragments, derivatives, conjugates, variants, and radioisotope-labeled complexes thereof, is administered to a subject by infusing a dose selected from the group consisting of about 10 mg, about 20 mg, about 25 mg, about 50 mg, about 75 mg, 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In an embodiment, the anti-CD20 antibody is administered weekly. In an embodiment, the anti-CD20 antibody is administered every two weeks. In an embodiment, the anti-CD20 antibody is administered every three weeks. In an embodiment, the anti-CD20 antibody is administered monthly. In an embodiment, the anti-CD20 antibody is administered at a lower initial dose, which is escalated when administered at subsequent intervals administered monthly. For example, the first infusion can deliver 300 mg of anti-CD20 antibody, and subsequent weekly doses could deliver 2,000 mg of anti-CD20 antibody for eight weeks, followed by monthly doses of 2,000 mg of anti-CD20 antibody. During any of the foregoing embodiments, the BTK inhibitors of the present invention and combinations of the BTK inhibitors with antifolate compounds may be administered daily, twice daily, or at different intervals as described above, at the dosages described above.

In an embodiment, the invention provides a kit comprising a first composition comprising a BTK inhibitor and a second composition comprising an antifolate compound and an anti-CD20 antibody selected from the group consisting of rituximab, obinutuzumab, ofatumumab, veltuzumab, tositumomab, and ibritumomab, or an antigen-binding fragment, derivative, conjugate, variant, or radioisotope-labeled complex thereof, for use in the treatment of CLL or SLL, hematological malignancies, B cell malignancies or, or any of the other diseases described herein. The compositions are typically both pharmaceutical compositions. The kit is for use in co-administration of the anti-CD20 antibody and the BTK inhibitor, either simultaneously or separately, in the treatment of CLL or SLL, hematological malignancies, B cell malignancies, or any of the other diseases described herein.

In an embodiment, the anti-CD20 monoclonal antibody is an anti-CD20 biosimilar monoclonal antibody approved by drug regulatory authorities with reference to rituximab, obinutuzumab, ofatumumab, veltuzumab, tositumomab, or ibritumomab. In an embodiment, the biosimilar comprises an anti-CD20 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is rituximab, obinutuzumab, ofatumumab, veltuzumab, tositumomab, or ibritumomab. In some embodiments, the one or more post-translational modifications are selected from one or more of: glycosylation, oxidation, deamidation, and truncation. In some embodiments, the biosimilar is an anti-CD20 antibody authorized or submitted for authorization, wherein the anti-CD20 antibody is provided in a formulation which differs from the formulations of a reference medicinal product or reference biological product, wherein the reference medicinal product or reference biological product is rituximab, obinutuzumab, ofatumumab, veltuzumab, tositumomab, or ibritumomab. The anti-CD20 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is rituximab, obinutuzumab, ofatumumab, veltuzumab, tositumomab, or ibritumomab. In some embodiments, the biosimilar comprises one or more excipients selected from tris-hydrochloride, sodium chloride, mannitol, pentetic acid, polysorbate 80, sodium hydroxide, and hydrochloric acid.

The anti-CD20 antibody sequences referenced in the foregoing are summarized in Table 1.

TABLE 1
Anti-CD20 antibody sequences.

| Identifier | Sequence (One-Letter Amino Acid Symbols) | |
|---|---|---|
| SEQ ID NO: 1 | QVQLQQPGAE LVKPGASVKM SCKASGYTFT SYNMHWVKQT PGRGLEWIGA IYPGNGDTSY | 60 |
| rjtuximab heavy | NQKFKGKATL TADKSSSTAY MQLSSLTSED SAVYYCARST YYGGDWYFNV WGAGTTVTVS | 120 |
| chain | AASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKKVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRD | 360 |
| | ELTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 2 | QIVLSQSPAI LSASPGEKVT MTCRASSSVS YIHWFQQKPG SSPKPWIYAT SNLASGVPVR | 60 |
| rituximab light | FSGSGSGTSY SLTISRVEAE DAATYYCQQW TSNPPTFGGG TKLEIKRTVA APSVFIFPPS | 120 |
| chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 3 | QVQLVQSGAE VKKPGSSVKV SCKASGYAFS YSWINWVRQA PGQGLEWMGR IFPGDGDTDY | 60 |
| obinutuzumab | NGKFKGRVTI TADKSTSTAY MELSSLRSED TAVYYCARNV FDGYWLVYWG QGTLVTVSSA | 120 |
| heavy chain | STKGPSVFPL APSSKSTSGG TAALGCLVKD YFPEPVTVSW NSGALTSGVH TFPAVLQSSG | 180 |
| | LYSLSSVVTV PSSSLGTQTY ICNVNHKPSN TKVDKKVEPK SCDKTHTCPP CPAPELLGGP | 240 |
| | SVFLFETKPK DTLMISRTPE VTCVVVDVSN EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 4 | DIVMTQTPLS LPVTPGEPAS ISCRSSKSLL HSNGITYLYW YLQKPGQSPQ LLIYQMSNLV | 60 |
| obinutuzumab | SGVPDRFSGS GSGTDFTLKI SRVEAEDVGV YYCAQNLELP YTFGGGTKVE IKRTVAAPSV | 120 |
| light chain | FIFPPSDEQL KSGTASVVCL LNNFYPREAK VQWKVDNALQ SGNSQESVTE QDSKDSTYSL | 180 |
| | SSTLTLSKAD YEKHKVYACE VTHQGLSSPV TKSFNRGEC | 219 |
| SEQ ID NO: 5 | EVQLVESGGG LVQPGRSLRL SCAASGFTFN DYAMHWVRQA PGKGLEWVST ISWNSGSIGY | 60 |
| ofatumumab | ADSVKGRFTI SRDNAKKSLY LQMNSLRAED TALYYCAKDI QYGNYYYGMD VWGQGTTVTV | 120 |
| variable heavy | SS | 122 |
| chain | | |
| SEQ ID NO: 6 | EIVLTQSPAT LSLSPGERAT LSCRASQSVS SYLAWYQQKP GQAPRLLIYD ASNRATGIPA | 60 |
| ofatumumab | RFSGSGSGTD FTLTISSLEP EDFAVYYCQQ RSNWPITFGQ GTRLEIK | 107 |
| variable light | | |
| chain | | |
| SEQ ID NO: 7 | EVQLVESGGG LVQPGRSLRL SCAASGFTFN DYAMHWVRQA PGKGLEWVST ISWNSGSIGY | 60 |
| ofatumumab Feb | ADSVKGRFTI SRDNAKKSLY LQMNSLRAED TALYYCAKDI QYGNYYYCMD VWGQGTTVTV | 120 |
| fragment heavy | SSASTKGPSV FPLAPGSSKS TSGTAALGCL VKDYFPEPVT VSWNSGALTS GVHTFPAVLQ | 180 |
| chain | SSGLYSLSSV VTVPSSSLGT QTYICNVNHK PSNTKVDKKV EP | 222 |
| SEQ ID NO: 8 | EIVLTQSPAT LSLSPGERAT LSCRASQSVS SYLAWYQQKP GQAPRLLIYD ASNRATGIPA | 60 |
| ofatumumab Fab | RFSGSGSGTD FTLTISSLEP EDFAVYYCQQ RSNWPITFGQ GTRLEIKRTV AAPSVFIFPP | 120 |
| fragment light | SDEQLKSGTA SVVOLLNNYY PREAKVQWKV DNALQSGNSQ ESVTEQDSKD STYSLSSTLT | 180 |
| chain | LSKADYEKHK VYACEVTHQG LSSPVTKSFN R | 211 |
| SEQ ID NO: 9 | QVQLQQSGAE VKKPGSSVKV SCKASGYTFT SYNMHWVKQA PGQGLEWIGA IYPGMGDTSY | 60 |
| veltuzumab heavy | NQKFKGKATL TADESTNTAY MELSSLRSED TAFYYCARST YYGGDWYFDV WGQGTTVTVS | 120 |
| chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVESCS VMHEALHNHY TQKSLSLSPG K | 451 |
| SEQ ID NO: 10 | DIQLTQSPSS LSASVGDRVT MTCRASSSVS YIHWFQQKPG KAPKPWIYAT SNLASGVPVR | 60 |
| veltuzumab light | FSGSGSGTDY TFTISSLQPE DIATYYCQQW TSNPPTFGGG TKLEIKRTVA APSVFIFPPS | 120 |
| chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 11 | QAYLQQSGAE LVRPGASVKM SCKASGYTFT SYNMHWVKQT PRQGLEWIGA IYPGNGDTSY | 60 |
| tositumomab | NQKFKGKATL TVDKSSSTAY MQLSSLTSED SAVYFCARVV YYSNSYWYFD VWGTGTTVTV | 120 |
| heavy chain | SGPSVFPLAP SSKSTSGGTA ALGCLVKDYF PEPVTVSWNS GALTSGVHTF PAVLQSSGLY | 180 |
| | SLSSVVTVPS SSLGTQTYIC NVNHKPSNTK VDKKAEPKSC DKTHTCPPCP APELLGGPSV | 240 |
| | FLFPPKPKDT LMISRTFEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSRDELTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID NO: 12 | QIVLSQSPAI LSASPGEKVT MTCRASSSVS YMHWYQQKPG SSPKPWIYAP SNLASGVPAR | 60 |
| tositumomab | FSGSGSGTSY SLTISRVEAE DAATYYCQQW SFNPPTFGAG TKLELKRTVA APSVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR | 210 |
| SEQ ID NO: 13 | QAYLQQSGAE LVRPGASVKM SCKASGYTFT SYNMHWVKQT PRQGLEWIGA IYPGNGDTSY | 60 |
| ibritumomab | NQKFKGKATL TVDKSSSTAY MQLSSLTSED SAVYFCARVV YYSNSYWYFD VWGTGTTVTV | 120 |
| heavy chain | SAPSVYPLAP VCGDTTGSSV TLGCLVKGYF PEPVTLTWNS GSLSSGVHTF PAVLQSDLYT | 180 |
| | LSSSVTVTSS TWPSQSITCN VAHPASSTKV DKKIEPRGPT IKPCPPCKCP APNLLGGPSV | 240 |
| | FIFPPKIKDV LMISLSPIVT CVVVDVSEDD PDVQISWFVN NVEVHTAQTQ THREDYNSTL | 300 |
| | RVVSALPIQH QDWMSGKEFK CKVNNKDLPA PIERTISKPK GSVRAPQVYV LPPPEEEMTK | 360 |
| | KQVTLTCMVT DFMPEDIYVE WTNNGKTELN YKNTEPVLDS DGSYFMYSKL RVEKKNWVER | 420 |
| | NSYSCSVVHE GLHNHHTTKS FSR | 443 |
| SEQ ID NO: 14 | QIVLSQSPAI LSASPGEKVT MTCRASSSVS YMHWYQQKPG SSPKPWIYAP SNLASGVPAR | 60 |
| ibritumomab | FSGSGSGTSY SLTISRVEAE DAATYYCQQW SFNPPTFGAG TKLELKRADA APTVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFN | 209 |

### Combinations of BTK Inhibitors, Antifolate Compounds, and PD-1 and PD-L1 Inhibitors

The combinations of the BTK inhibitors with antifolate compounds may also be further combined with programmed death-1 (PD-1), programmed death ligand 1 (PD-L1), and/or programmed death ligand 2 (PD-L2) binding antibodies or inhibitors (i.e., blockers). In a preferred embodiment, the PD-1 or PD-L1 inhibitor for use in combination with an antifolate compound and a BTK inhibitor is selected from the group consisting of nivolumab, pembrolizumab, pidilizumab, durvalumab, atezolizumab, avelumab, and antigen-binding fragments, variants, conjugates, or biosimilars thereof. In a preferred embodiment, the invention provides a method of treating a cancer or an inflammatory, immune, autoimmune disorder in a human comprising the step of administering to said human a BTK inhibitor, or a pharmaceutically acceptable salt, solvate, hydrate, cocrystal, or prodrug thereof, and an antifolate compound or a pharmaceutically acceptable salt, solvate, hydrate, cocrystal, or prodrug thereof, and further comprising the step of administering an PD-1 or PD-L inhibitor, or an antigen-binding fragment, derivative, conjugate, variant, or biosimilar thereof. In an embodiment, the BTK inhibitor is a compound selected from the group consisting of Formula (1), Formula (2), Formula (3), Formula (4), Formula (5), Formula (6), Formula (7), Formula (10), and Formula (21), and pharmaceutically acceptable salts, solvates, hydrates, cocrystals, or prodrugs thereof.

Programmed death 1 (PD-1) is a 288-amino acid transmembrane immunocheckpoint receptor protein expressed by T cells, B cells, natural killer (NK) T cells, activated monocytes, and dendritic cells. PD-1, which is also known as CD279, is an immunoreceptor belonging to the CD28 family and in humans is encoded by the Pdcd1 gene on chromosome 2. PD-1 consists of one immunoglobulin (Ig) superfamily domain, a transmembrane region, and an intracellular domain containing an immunoreceptor tyrosine-based inhibitory motif (ITIM) and an immunoreceptor tyrosine-based switch motif (ITSM). PD-1 and its ligands (PD-L1 and PD-L2) play a key role in immune tolerance, as described in Keir, et al., Annu. Rev. Immunol. 2008, 26, 677-704. PD-1 provides inhibitory signals that negatively regulate T cell immune responses. PD-L1 (also known as B7-H1 or CD274) and PD-L2 (also known as B7-DC or CD273) are expressed on tumor cells and stromal cells, which may be encountered by activated T cells expressing PD-1, leading to immunosuppression of the T cells. PD-L1 is a 290 amino acid transmembrane protein encoded by the Cd274 gene on human chromosome 9. Blocking the interaction between PD-1 and its ligands PD-L1 and PD-L2 by use of a PD-1 inhibitor, a PD-L1 inhibitor, and/or a PD-L2 inhibitor can overcome immune resistance, as demonstrated in recent clinical studies, such as that described in Topalian, et al., N. Eng. J. Med. 2012, 366, 2443-54. PD-L1 is expressed on many tumor cell lines, while PD-L2 is expressed is expressed mostly on dendritic cells and a few tumor lines. In addition to T cells (which inducibly express PD-1 after activation), PD-1 is also expressed on B cells, natural killer cells, macrophages, activated monocytes, and dendritic cells.

In an embodiment, the PD-1 inhibitor may be any PD-1 inhibitor or PD-1 blocker known in the art. In particular, it is one of the PD-1 inhibitors or blockers described in more detail in the following paragraphs. The terms “inhibitor” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a compound or a pharmaceutically acceptable salt, ester, solvate, hydrate, cocrystal, or prodrug thereof.

In some embodiments, the compositions and methods described herein include a PD-1 inhibitor. In some embodiments, the PD-1 inhibitor is a small molecule. In a preferred embodiment, the PD-1 inhibitor is an antibody (i.e., an anti-PD-1 antibody), a fragment thereof, including Fab fragments, or a single-chain variable fragment (scFv) thereof. In some embodiments the PD-1 inhibitor is a polyclonal antibody. In a preferred embodiment, the PD-1 inhibitor is a monoclonal antibody. In some embodiments, the PD-1 inhibitor competes for binding with PD-1, and/or binds to an epitope on PD-1. In an embodiment, the antibody competes for binding with PD-1, and/or binds to an epitope on PD-1. In some embodiments, an anti-PD-1 monoclonal antibody is included in a composition or a method and is further combined with a BTK inhibitor and/or an antifolate compound. In some embodiments, a PD-1 inhibitor is included in a composition or a method and is further combined with a BTK inhibitor. In some embodiments, an anti-PD-1 monoclonal antibody is included in a composition or a method and is further combined with a BTK inhibitor. In some embodiments, a PD-1 inhibitor is included in a composition or a method and is further combined with an antifolate compound. In some embodiments, an anti-PD-1 monoclonal antibody is included in a composition or a method and is further combined with an antifolate compound. In preferred embodiments, the compositions described herein provide a combination of a PD-1 inhibitor with a BTK inhibitor, or methods of using a combination of a PD-1 inhibitor with a BTK inhibitor. In some embodiments, the PD-1 inhibitors provided herein are selective for PD-1, in that the compounds bind or interact with PD-1 at substantially lower concentrations than they bind or interact with other receptors.

In some embodiments, the compositions and methods described include a PD-1 inhibitor that binds human PD-1 with a K_{D} of about 100 pM or lower, binds human PD-1 with a K_{D} of about 90 pM or lower, binds human PD-1 with a K_{D} of about 80 pM or lower, binds human PD-1 with a K_{D} of about 70 pM or lower, binds human PD-1 with a K_{D} of about 60 pM or lower, binds human PD-1 with a K₀ of about 50 pM or lower, binds human PD-1 with a K_{D} of about 40 pM or lower, binds human PD-1 with a K_{D} of about 30 pM or lower, binds human PD-1 with a K_{D} of about 20 pM or lower, binds human PD-1 with a K_{D} of about 10 pM or lower, or binds human PD-1 with a K_{D} of about 1 pM or lower.

In some embodiments, the compositions and methods described include a PD-1 inhibitor that binds to human PD-1 with a k_{assoc} of about 7.5×10⁵ l/M·s or faster, binds to human PD-1 with a k_{assoc} of about 7.5×10⁵ l/M·s or faster, binds to human PD-1 with a k_{assoc} of about 8×10⁵ l/M·s or faster, binds to human PD-1 with a k_{assoc} of about 8.5×10⁵ l/M·s or faster, binds to human PD-1 with a k_{assoc} of about 9×10⁵ l/M·s or faster, binds to human PD-1 with a k_{assoc} of about 9.5×10⁵ l/M·s or faster, or binds to human PD-1 with a k_{assoc} of about 1×10⁶ l/M·s or faster.

In some embodiments, the compositions and methods described include a PD-1 inhibitor that binds to human PD-1 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower or binds to human PD-1 with a k_{dissoc} of about 2.7×10⁻⁵ l/s or slower, binds to human PD-1 with a k_{dissoc} of about 2.8×10⁻⁵ l/s or slower, binds to human PD-1 with a k_{dissoc} of about 2.9×10⁻⁵ l/s or slower, or binds to human PD-1 with a k_{dissoc} of about 3×10⁻⁵ l/s or slower.

In some embodiments, the compositions and methods described include a PD-1 inhibitor that blocks or inhibits binding of human PD-L1 or human PD-L2 to human PD-1 with an IC₅₀ of about 10 nM or lower, blocks or inhibits binding of human PD-L1 or human PD-L2 to human PD-1 with an IC₅₀ of about 9 nM or lower, blocks or inhibits binding of human PD-L1 or human PD-L2 to human PD-1 with an IC₅₀ of about 8 nM or lower, blocks or inhibits binding of human PD-L1 or human PD-L2 to human PD-1 with an IC₅₀ of about 7 nM or lower, blocks or inhibits binding of human PD-L1 or human PD-L2 to human PD-1 with an IC₅₀ of about 6 nM or lower, blocks or inhibits binding of human PD-L1 or human PD-L2 to human PD-1 with an IC₅₀ of about 5 nM or lower, blocks or inhibits binding of human PD-L1 or human PD-L2 to human PD-1 with an IC₅₀ of about 4 nM or lower, blocks or inhibits binding of human PD-L1 or human PD-L2 to human PD-1 with an IC₅₀ of about 3 nM or lower, blocks or inhibits binding of human PD-L1 or human PD-L2 to human PD-1 with an IC₅₀ of about 2 nM or lower, or blocks or inhibits binding of human PD-L1 or human PD-L2 to human PD-1 with an IC₅₀ of about 1 nM or lower.

In an embodiment, an anti-PD-1 antibody comprises nivolumab (also known as OPDIVO and commercially available from Bristol-Myers Squibb Co.), or biosimilars, antigen-binding fragments, conjugates, or variants thereof. Nivolumab is referred to as 5C4 in International Patent Publication No. WO 2006/121168. Nivolumab is assigned Chemical Abstracts Service (CAS) registry number 946414-94-4 and is also known as BMS-936558, MDX-1106 or ONO-4538. Nivolumab is a fully human IgG4 antibody blocking the PD-1 receptor. The clinical safety and efficacy of nivolumab in various forms of cancer has been described in Wang et al., Cancer Immunol Res. 2014, 2, 846-56; Page et al., Ann. Rev. Med., 2014, 65, 185-202; and Weber, et al., J. Clin. Oncology, 2013, 31, 4311-4318. The nivolumab monoclonal antibody includes a heavy chain given by SEQ ID NO: 15 and a light chain given by SEQ ID NO:16. Nivolumab has intra-heavy chain disulfide linkages at 22-96, 140-196, 254-314, 360-418, 22″-96″, 140″-196″, 254″-314″, and 360″-418″; intra-light chain disulfide linkages at 23′-88′, 134′-194′, 23′″-88′″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH₂ 84.4) at 290, 290″. In an embodiment, the anti-PD-1 antibody is an immunoglobulin G4 kappa, anti-(human CD274) antibody. In an embodiment, an anti-PD-1 antibody comprises heavy and light chains having the sequences shown in SEQ ID NO: 15 and SEQ ID NO: 16, respectively, or antigen binding fragments, Fab fragments, single-chain variable fragments (scFv), variants, or conjugates thereof. In an embodiment, an anti-PD-1 antibody comprises heavy and light chains that are each at least 99% identical to the sequences shown in SEQ ID NO: 15 and SEQ ID NO: 16, respectively. In an embodiment, an anti-PD-1 antibody comprises heavy and light chains that are each at least 98% identical to the sequences shown in SEQ ID NO: 15 and SEQ ID NO:16, respectively. In an embodiment, an anti-PD-1 antibody comprises heavy and light chains that are each at least 97% identical to the sequences shown in SEQ ID NO:15 and SEQ ID NO:16, respectively. In an embodiment, an anti-PD-1 antibody comprises heavy and light chains that are each at least 96% identical to the sequences shown in SEQ ID NO: 15 and SEQ ID NO: 16, respectively. In an embodiment, an anti-PD-1 antibody comprises heavy and light chains that are each at least 95% identical to the sequences shown in SEQ ID NO:15 and SEQ ID NO:16, respectively.

In an embodiment, the anti-PD-1 antibody is an anti-PD-1 biosimilar monoclonal antibody approved by drug regulatory authorities with reference to nivolumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

In an embodiment, the anti-PD-1 antibody comprises the heavy and light chain CDRs or variable regions (VRs) of nivolumab. In one embodiment, the anti-PD-1 antibody heavy chain variable region (V_{H}) comprises the sequence shown in SEQ ID NO:17, and the anti-PD-1 antibody light chain variable region (V_{L}) comprises the sequence shown in SEQ ID NO:18. In an embodiment, an anti-PD-1 antibody comprises V_{H} and V_{L} regions that are each at least 99% identical to the sequences shown in SEQ ID NO:17 and SEQ ID NO:18, respectively. In an embodiment, an anti-PD-1 antibody comprises V_{H} and V_{L} regions that are each at least 98% identical to the sequences shown in SEQ ID NO:17 and SEQ ID NO: 18, respectively. In an embodiment, an anti-PD-1 antibody comprises V_{H} and V_{L} regions that are each at least 97% identical to the sequences shown in SEQ ID NO: 17 and SEQ ID NO: 18, respectively. In an embodiment, an anti-PD-1 antibody comprises V_{H} and V_{L} regions that are each at least 96% identical to the sequences shown in SEQ ID NO:17 and SEQ ID NO:18, respectively. In an embodiment, an anti-PD-1 antibody comprises V_{H} and V_{L} regions that are each at least 95% identical to the sequences shown in SEQ ID NO: 17 and SEQ ID NO: 18, respectively. In an alternative embodiment, the antibody comprises V_{H} and/or V_{L} regions having the amino acid sequences set forth in SEQ ID NO:17 and/or SEQ ID NO:18, respectively.

In an embodiment, the anti-PD-1 antibody comprises heavy chain CDR1, CDR2 and CDR3 domains having the sequences set forth in SEQ ID NO: 19, SEQ ID NO:20, and SEQ ID NO:21, respectively, and light chain CDR1, CDR2 and CDR3 domains having the sequences set forth in SEQ ID NO:22, SEQ ID NO:23, and SEQ ID NO:24, respectively.

In an embodiment, an anti-PD-1 antibody comprises a heavy chain that comprises CDR1, CDR2 and CDR3 domains that are each at least 95% identical to the sequences shown in SEQ ID NO:19, SEQ ID NO:20, and SEQ ID NO:21, respectively. In an embodiment, an anti-PD-1 antibody comprises a heavy chain that comprises CDR1, CDR2 and CDR3 domains that are each at least 90% identical to the sequences shown in SEQ ID NO:19, SEQ ID NO:20, and SEQ ID NO:21, respectively. In an embodiment, an anti-PD-1 antibody comprises a heavy chain that comprises CDR1, CDR2 and CDR3 domains that are each at least 85% identical to the sequences shown in SEQ ID NO: 19, SEQ ID NO:20, and SEQ ID NO:21, respectively. In an embodiment, an anti-PD-1 antibody comprises a heavy chain that comprises CDR1, CDR2 and CDR3 domains that are each at least 80% identical to the sequences shown in SEQ ID NO:19, SEQ ID NO:20, and SEQ ID NO:21, respectively. In another embodiment, the antibody competes for binding with, and/or binds to the same epitope on PD-1 as the aforementioned antibodies.

In an embodiment, an anti-PD-1 antibody comprises a light chain that comprises CDR1, CDR2 and CDR3 domains that are each at least 95% identical to the sequences shown in SEQ ID NO:22, SEQ ID NO:23, and SEQ ID NO:24, respectively. In an embodiment, an anti-PD-1 antibody comprises a light chain that comprises CDR1, CDR2 and CDR3 domains that are each at least 90% identical to the sequences shown in SEQ ID NO:22, SEQ ID NO:23, and SEQ ID NO:24, respectively. In an embodiment, an anti-PD-1 antibody comprises a light chain that comprises CDR1, CDR2 and CDR3 domains that are each at least 85% identical to the sequences shown in SEQ ID NO:22, SEQ ID NO:23, and SEQ ID NO:24, respectively. In an embodiment, an anti-PD-1 antibody comprises a light chain that comprises CDR1, CDR2 and CDR3 domains that are each at least 80% identical to the sequences shown in SEQ ID NO:22, SEQ ID NO:23, and SEQ ID NO:24, respectively. In another embodiment, the antibody competes for binding with, and/or binds to the same epitope on PD-1 as the aforementioned antibodies.

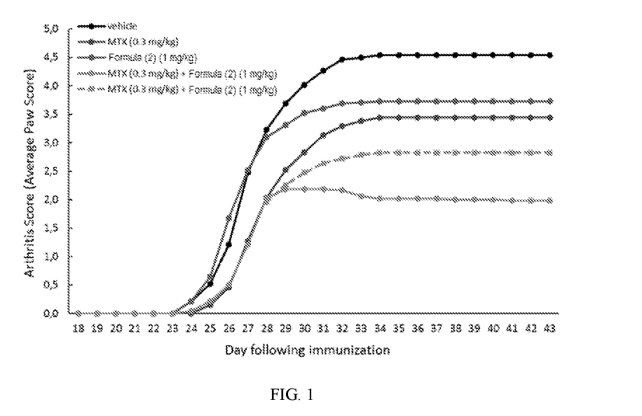

In an embodiment, the anti-PD-1 antibody is an antibody disclosed and/or prepared according to U.S. Pat. No. 8,008,449 or U.S. Patent Application Publication Nos. 2009/0217401 A1 or 2013/0133091 A1, the disclosures of which are specifically incorporated by reference herein. For example, in an embodiment, the monoclonal antibody includes 5C4 (referred to herein as nivolumab), 17D8, 2D3, 4H1, 4A11, 7D3, and 5F4, described in U.S. Pat. No. 8,008,449, the disclosures of which are hereby incorporated by reference. The PD-1 antibodies 17D8, 2D3, 4H1, 5C4, and 4A11, are all directed against human PD-1, bind specifically to PD-1 and do not bind to other members of the CD28 family. The sequences and CDR regions for these antibodies are provided in U.S. Pat. No. 8,008,449, in particular in FIG. 1 through FIG. 12 therein; the disclosures of which are incorporated by reference herein.

The anti-PD-1 antibody nivolumab may be prepared by the following procedure, as described in U.S. Pat. No. 8,008,449. Immunization protocols utilized as antigen both (i) a recombinant fusion protein comprising the extracellular portion of PD-1 and (ii) membrane bound full-length PD-1. Both antigens were generated by recombinant transfection methods in a CHO cell line. Fully human monoclonal antibodies to PD-1 were prepared using the HCo7 strain of HuMab transgenic mice and the KM strain of transgenic transchromosomic mice, each of which express human antibody genes. In each of these mouse strains, the endogenous mouse kappa light chain gene has been homozygously disrupted as described in Chen, et al. EMBO J. 1993, 12, 811-820 and the endogenous mouse heavy chain gene has been homozygously disrupted as described in Example 1 of International Patent Publication No. WO 01/09187. Each of these mouse strains carries a human kappa light chain transgene, KCoS, as described in Fishwild, et al. Nat. Biotechnology 1996, 14, 845-851. The HCo7 strain carries the HCo7 human heavy chain transgene as described in U.S. Pat. Nos. 5,545,806; 5,625,825; and 5,545,807. The KM strain contains the SC20 transchromosome as described in International Patent Publication No. WO 02/43478. To generate fully human monoclonal antibodies to PD-1, HuMab mice and KM Mice™ were immunized with purified recombinant PD-1 fusion protein and PD-1-transfected CHO cells as antigen. General immunization schemes for HuMab mice are described in Lonberg, et al., Nature 1994, 368, 856-859; Fishwild, et al., Nat. Biotechnology 1996, 14, 845-851, and International Patent Publication No. WO 98/24884. The mice were 6-16 weeks of age upon the first infusion of antigen. A purified recombinant preparation (5-50 μg) of PD-1 fusion protein antigen and 5-10×10⁶ cells were used to immunize the HuMab mice and KM Mice™ intraperitonealy, subcutaneously (Sc) or via footpad injection. Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA (as described below), and mice with sufficient titers of anti-PD-1 human immunoglobulin were used for fusions. Mice were boosted intravenously with antigen 3 days before sacrifice and removal of the spleen. Typically, 10-35 fusions for each antigen were performed. Several dozen mice were immunized for each antigen. To select HuMab or KM Mice™ producing antibodies that bound PD-1, sera from immunized mice were tested by ELISA as described by Fishwild, et al., Nat. Biotechnology 1996, 14, 845-851. Briefly, microtiter plates were coated with purified recombinant PD-1 fusion protein from transfected CHO cells at 1-2 μg/ml in PBS, 100 μL/wells incubated at 4° C. overnight then blocked with 200 μL/well of 5% fetal bovine serum in PBS/Tween (0.05%). Dilutions of sera from PD-1-immunized mice were added to each well and incubated for 1-2 hours at ambient temperature. The plates were washed with PBS/Tween and then incubated with a goat-anti-human IgG polyclonal antibody conjugated with horseradish peroxidase (HRP) for 1 hour at room temperature. After washing, the plates were developed with ABTS substrate (Sigma, A-1888, 0.22 mg/ml) and analyzed by spectrophotometer at OD 415-495. Mice that developed the highest titers of anti-PD-1 antibodies were used for fusions. Fusions were performed as described below and hybridoma supernatants were tested for anti-PD-1 activity by ELISA. The mouse splenocytes, isolated from the HuMab or KM mice, were fused to a mouse myeloma cell line either using PEG based upon standard protocols or electric field based electrofusion using a Cyto Pulse large chamber cell fusion electroporator (Cyto Pulse Sciences, Inc., Glen Burnie, Md.). The resulting hybridomas were then screened for the production of antigen-specific antibodies. Single cell suspensions of splenocytes from immunized mice were fused to one-fourth the number of SP2/0 nonsecreting mouse myeloma cells (ATCC, CRL 1581) with 50% PEG (Sigma). Cells were plated at approximately 1×105/well in flat bottom microtiter plate, followed by about two week incubation in selective medium containing 10% fetal bovine serum, 10% P388D1 (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After 1-2 weeks, cells were cultured in medium in which the HAT was replaced with HT. Individual wells were then screened by ELISA (described above) for human anti-PD-1 monoclonal IgG antibodies. Once extensive hybridoma growth occurred, medium was monitored usually after 10-14 days. The antibody-secreting hybridomas were replated, screened again and, if still positive for human IgG, anti-PD-1 monoclonal antibodies were subcloned at least twice by limiting dilution. The stable subclones were then cultured in vitro to generate small amounts of antibody in tissue culture medium for further characterization. The antibody nivolumab may be produced in this manner, or by other known means given the disclosure of the amino acid sequences herein.

In another embodiment, the anti-PD-1 antibody comprises pembrolizumab (also known as KEYTRUDA), which is commercially available from Merck, or antigen-binding fragments, conjugates, or variants thereof. Pembrolizumab is assigned CAS registry number 1374853-91-4 and is also known as lambrolizumab, MK-3475, and SCH-900475. The structure, properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal κ light chain dimer (226-226″:229-229″)-bisdisulfide. The clinical safety and efficacy of pembrolizumab in various forms of cancer is described in Fuerst, Oncology Times, 2014, 36, 35-36; Robert, et al., Lancet, 2014, 384, 1109-17; and Thomas, et al., Exp. Opin. Biol. Ther., 2014, 14, 1061-1064. In an embodiment, the pembrolizumab monoclonal antibody includes a heavy chain given by SEQ ID NO:25 and a light chain given by SEQ ID NO:26, and includes the following disulfide bridges: 22-96, 22″-96″, 23′-92′, 23′″-92′″, 134-218′, 134″-218′″, 138′-198′, 138′″-198′″, 147-203, 147″-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (G0F).

In an embodiment, an anti-PD-1 antibody comprises heavy and light chains having the sequences shown in SEQ ID NO:25 and SEQ ID NO:26, respectively, or antigen binding fragments and variants thereof. In an embodiment, an anti-PD-1 antibody comprises heavy and light chains that are each at least 99% identical to the sequences shown in SEQ ID NO:25 and SEQ ID NO:26, respectively, or antigen binding fragments and variants thereof. In an embodiment, an anti-PD-1 antibody comprises heavy and light chains that are each at least 98% identical to the sequences shown in SEQ ID NO:25 and SEQ ID NO:26, respectively, or antigen binding fragments and variants thereof. In an embodiment, an anti-PD-1 antibody comprises heavy and light chains that are each at least 97% identical to the sequences shown in SEQ ID NO:25 and SEQ ID NO:26, respectively, or antigen binding fragments and variants thereof. In an embodiment, an anti-PD-1 antibody comprises heavy and light chains that are each at least 96% identical to the sequences shown in SEQ ID NO:25 and SEQ ID NO:26, respectively, or antigen binding fragments and variants thereof. In an embodiment, an anti-PD-1 antibody comprises heavy and light chains that are each at least 95% identical to the sequences shown in SEQ ID NO:25 and SEQ ID NO:26, respectively, or antigen binding fragments and variants thereof.

In an embodiment, the anti-PD-1 antibody is an anti-PD-1 biosimilar monoclonal antibody approved by drug regulatory authorities with reference to pembrolizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

In an embodiment, the anti-PD-1 antibody comprises the heavy and light chain CDRs or VRs of pembrolizumab. In one embodiment, the anti-PD-1 antibody V_{H} region comprises the sequence shown in SEQ ID NO:27, and the anti-PD-1 antibody V_{L} region comprises the sequence shown in SEQ ID NO:28. In an embodiment, an anti-PD-1 antibody comprises V_{H} and V_{L} regions that are each at least 99% identical to the sequences shown in SEQ ID NO:27 and SEQ ID NO:28, respectively. In an embodiment, an anti-PD-1 antibody comprises V_{H} and V_{L} regions that are each at least 98% identical to the sequences shown in SEQ ID NO:27 and SEQ ID NO:28, respectively. In an embodiment, an anti-PD-1 antibody comprises V_{H} and V_{L} regions that are each at least 97% identical to the sequences shown in SEQ ID NO:27 and SEQ ID NO:28, respectively. In an embodiment, an anti-PD-1 antibody comprises V_{H} and V_{L} regions that are each at least 96% identical to the sequences shown in SEQ ID NO:27 and SEQ ID NO:28, respectively. In an embodiment, an anti-PD-1 antibody comprises V_{H} and V_{L} regions that are each at least 95% identical to the sequences shown in SEQ ID NO:27 and SEQ ID NO:28, respectively. In an alternative embodiment, the antibody comprises V_{H} and/or V_{L} regions having the amino acid sequences set forth in SEQ ID NO:27 and/or SEQ ID NO:28, respectively.

In an embodiment, the anti-PD-1 antibody comprises heavy chain CDR1, CDR2 and CDR3 domains having the sequences set forth in SEQ ID NO:29, SEQ ID NO:30, and SEQ ID NO:31, respectively, and light chain CDR1, CDR2 and CDR3 domains having the sequences set forth in SEQ ID NO:32, SEQ ID NO:33, and SEQ ID NO:34, respectively.

In an embodiment, an anti-PD-1 antibody comprises a heavy chain that comprises CDR1, CDR2 and CDR3 domains that are each at least 95% identical to the sequences shown in SEQ ID NO:29, SEQ ID NO:30, and SEQ ID NO:31, respectively. In an embodiment, an anti-PD-1 antibody comprises a heavy chain that comprises CDR1, CDR2 and CDR3 domains that are each at least 90% identical to the sequences shown in SEQ ID NO:29, SEQ ID NO:30, and SEQ ID NO:31, respectively. In an embodiment, an anti-PD-1 antibody comprises a heavy chain that comprises CDR1, CDR2 and CDR3 domains that are each at least 85% identical to the sequences shown in SEQ ID NO:29, SEQ ID NO:30, and SEQ ID NO:31, respectively. In an embodiment, an anti-PD-1 antibody comprises a heavy chain that comprises CDR1, CDR2 and CDR3 domains that are each at least 80% identical to the sequences shown in SEQ ID NO:29, SEQ ID NO:30, and SEQ ID NO:31, respectively. In another embodiment, the antibody competes for binding with, and/or binds to the same epitope on PD-1 as the aforementioned antibodies.

In an embodiment, an anti-PD-1 antibody comprises a light chain that comprises CDR1, CDR2 and CDR3 domains that are each at least 95% identical to the sequences shown in SEQ ID NO:32, SEQ ID NO:33, and SEQ ID NO:34, respectively. In an embodiment, an anti-PD-1 antibody comprises a light chain that comprises CDR1, CDR2 and CDR3 domains that are each at least 90% identical to the sequences shown in SEQ ID NO:32, SEQ ID NO:33, and SEQ ID NO:34, respectively. In an embodiment, an anti-PD-1 antibody comprises a light chain that comprises CDR1, CDR2 and CDR3 domains that are each at least 85% identical to the sequences shown in SEQ ID NO:32, SEQ ID NO:33, and SEQ ID NO:34, respectively. In an embodiment, an anti-PD-1 antibody comprises a light chain that comprises CDR1, CDR2 and CDR3 domains that are each at least 80% identical to the sequences shown in SEQ ID NO:32, SEQ ID NO:33, and SEQ ID NO:34, respectively. In another embodiment, the antibody competes for binding with, and/or binds to the same epitope on PD-1 as the aforementioned antibodies.

In an embodiment, the anti-PD-1 antibody is an antibody disclosed in U.S. Pat. No. 8,354,509 or U.S. Patent Application Publication Nos. 2010/0266617 A1, 2013/0108651 A1, 2013/0109843 A2, the disclosures of which are specifically incorporated by reference herein.

In an embodiment, the anti-PD-1 antibody is pidilizumab, which is also known as CT-011 (CureTech Ltd.), and which is disclosed in U.S. Pat. No. 8,686,119 B2, the disclosures of which are specifically incorporated by reference herein. The efficacy of pidilizumab in the treatment of cancers, such as hematological malignancies, is described in Berger, et al., Clin. Cancer Res. 2008, 14, 3044-51. The pidilizumab monoclonal antibody includes a heavy chain given by SEQ ID NO:35 and a light chain given by SEQ ID NO:36. Pidilizumab has intra-heavy chain disulfide linkages at 22-96, 144-200, 261-321, 367-425, 22″-96″, 144″-200″, 261″-321″, and 367″-425″; intra-light chain disulfide linkages at 23′-87′, 133′-193′, 23′″-87′″, and 133′″-193′″; inter-heavy-light chain disulfide linkages at 220-213′ and 220″-213′″, inter-heavy-heavy chain disulfide linkages at 226-226″ 229-229″; and N-glycosylation sites (H CH₂ 84.4) at 297, 297″.

In an embodiment, the anti-PD-1 antibody is an immunoglobulin G1 kappa, anti-(human CD274) humanized monoclonal antibody. In an embodiment, an anti-PD-1 antibody comprises heavy and light chains having the sequences shown in SEQ ID NO:35 and SEQ ID NO:36, respectively, or antigen binding fragments, variants, or conjugates thereof. In an embodiment, an anti-PD-1 antibody comprises heavy and light chains that are each at least 99% identical to the sequences shown in SEQ ID NO:35 and SEQ ID NO:36, respectively. In an embodiment, an anti-PD-1 antibody comprises heavy and light chains that are each at least 98% identical to the sequences shown in SEQ ID NO:35 and SEQ ID NO:36, respectively. In an embodiment, an anti-PD-1 antibody comprises heavy and light chains that are each at least 97% identical to the sequences shown in SEQ ID NO:35 and SEQ ID NO:36, respectively. In an embodiment, an anti-PD-1 antibody comprises heavy and light chains that are each at least 96% identical to the sequences shown in SEQ ID NO:35 and SEQ ID NO:36, respectively. In an embodiment, an anti-PD-1 antibody comprises heavy and light chains that are each at least 95% identical to the sequences shown in SEQ ID NO:35 and SEQ ID NO:36, respectively.

In an embodiment, the anti-PD-1 antibody is an anti-PD-1 biosimilar monoclonal antibody approved by drug regulatory authorities with reference to pidilizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pidilizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pidilizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pidilizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pidilizumab.

In an embodiment, an anti-PD-L1 antibody comprises V_{H} and V_{L} regions that are each at least 99% identical to the sequences shown in SEQ ID NO:37 and SEQ ID NO:38, respectively. In an embodiment, an anti-PD-L1 antibody comprises V_{H} and V_{L} regions that are each at least 98% identical to the sequences shown in SEQ ID NO:37 and SEQ ID NO:38, respectively. In an embodiment, an anti-PD-L1 antibody comprises V_{H} and V_{L} regions that are each at least 97% identical to the sequences shown in SEQ ID NO:37 and SEQ ID NO:38, respectively. In an embodiment, an anti-PD-L1 antibody comprises V_{H} and V_{L} regions that are each at least 96% identical to the sequences shown in SEQ ID NO:37 and SEQ ID NO:38, respectively. In an embodiment, an anti-PD-L1 antibody comprises V_{H} and V_{L} regions that are each at least 95% identical to the sequences shown in SEQ ID NO:37 and SEQ ID NO:38, respectively.

In an embodiment, anti-PD-1 antibodies and other PD-1 inhibitors include those described in U.S. Pat. Nos. 8,287,856, 8,580,247, and 8,168,757 and U.S. Patent Application Publication Nos. 2009/0028857 A1, 2010/0285013 A1, 2013/0022600 A1, and 2011/0008369 A1, the teachings of which are hereby incorporated by reference. In another embodiment, antibodies that compete with any of these antibodies for binding to PD-1 are also included. In another embodiment, the anti-PD-1 antibody is an antibody disclosed in U.S. Pat. No. 8,735,553 B1, the disclosures of which are incorporated herein by reference.

In an embodiment, the anti-PD-1 antibody is a commercially-available monoclonal antibody, such as anti-m-PD-1 clones J43 (Cat # BE0033-2) and RMP1-14 (Cat #BE0146) (Bio X Cell, Inc., West Lebanon, N.H., USA). A number of commercially-available anti-PD-1 antibodies are known to one of ordinary skill in the art.

Monoclonal antibodies that inhibit or block PD-1 can be prepared by procedures known to those of ordinary knowledge and skill in the art, e.g., by injecting test subjects with PD-1 antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, myeloma cells, or other suitable cells that do not otherwise produce immunoglobulin protein, to obtain the synthesis of monoclonal antibodies in the recombinant host cells. The details of recombinant production of specific antibodies may be found in the references cited in the foregoing, the disclosures of which are incorporated by reference herein. Monoclonal antibodies that inhibit PD-1 can be prepared by standard molecular biology methods using the sequences provided herein by reverse translation and insertion into appropriate DNA or RNA vectors.

In an embodiment, the PD-1 inhibitor may be a small molecule or a peptide, or a peptide derivative, such as those described in U.S. Pat. Nos. 8,907,053; 9,096,642; and 9,044,442 and U.S. Patent Application Publication No. US 2015/0087581; 1,2,4-oxadiazole compounds and derivatives such as those described in U.S. Patent Application Publication No. 2015/0073024; cyclic peptidomimetic compounds and derivatives such as those described in U.S. Patent Application Publication No. US 2015/0073042; cyclic compounds and derivatives such as those described in U.S. Patent Application Publication No. US 2015/0125491; 1,3,4-oxadiazole and 1,3,4-thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. US 2014/0294898; the disclosures of each of which are hereby incorporated by reference in their entireties.

The anti-PD-1 antibody sequences discussed and referenced in some of the foregoing embodiments are summarized in Table 2.

TABLE 2
Anti-PD-1 antibody amino acid sequences.

| Identifier | Sequence (One-Letter Amino Acid Symbols) | |
|---|---|---|
| SEQ ID NO: 18 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSSASTKGPS | 120 |
| heavy chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP PCPAPEFLGG PSVFLEPPEP | 240 |
| | KDTLMISRTP EVTCVVVDVS QEDPEVQFNW YVDGVEVHNA KTEPREEQFN STYRVVSVLT | 300 |
| | VLHQDWLNGK EYKCKVSNKG LPSSIEKTIS KAKGQPREPQ VYTLPPSQEE MTKNQVSLTC | 360 |
| | LVKGFYPSDI AVEWESNGQP ENNYKTTPPV LDSDGSFFLY SRLTVDKSRW QEGNVFSCSV | 420 |
| | MHEALHNHYT QKLSLSLGK | 440 |
| SEQ ID NO: 16 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIKRTV AAPSVFIFPP | 120 |
| light chain | SDEQLKSGTA SVVCLLNNFY PREAKVOWKV DNALQSGNSQ ESVTEQDSKD STYSLSSTLT | 180 |
| | LSKADYEKHK VYACEVTHQG LSSPVTKSFN RGEC | 214 |
| SEQ ID NO: 17 | QVQLVESGGG VVQPORSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSS | 113 |
| variable heavy | | |
| chain | | |
| SEQ ID NO: 18 | EIVLTQSPAT LSLSPGERAT LSCRASQSVS SYLAWYQQKP GQAPPLLIYD ASNRATGIPA | 60 |
| nivolumab | RFSGSGSGTD FTLTISSLEP EDFAVYYCQQ SSNWPRTFGQ GTKVEIK | 107 |
| variable light | | |
| chain | | |
| SEQ ID 140: 19 | NSGMH | 5 |
| nivolumab | | |
| heavy chain | | |
| CDR1 | | |
| SEQ ID NO: 20 | VIWYDGSKRY YADSVKG | 16 |
| nivolumab | | |
| heavy chain | | |
| CDR2 | | |
| SEQ ID NO: 21 | EDDY | 4 |
| nivolumab | | |
| heavy chain | | |
| CDR3 | | |
| SEQ ID NO: 22 | RASQSVSSYL A | 11 |
| nivolumab | | |
| light chain | | |
| CDR1 | | |
| SEQ ID NO: 23 | DASNRAT | 7 |
| nivolumab | | |
| light chain | | |
| CDR2 | | |
| SEQ ID NO: 24 | QQSSNWPRT | 9 |
| nivolumab | | |
| light chain | | |
| CDR3 | | |
| SEQ ID NO: 25 | OVOLVOSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVLTVLH QDWLNGKEYK CKVSNKGLPS SIEKTISKAK GQPREPQVYT LPPSQEEMTK | 360 |
| | NQVSLTCLVK GFYPSDIAVE WESNGQPENN YKTTPPVLDS DGSFFLYSRL TVDKSRWQEG | 420 |
| | NVFSCSVMHE ALHNHYTQKS LSLSLGK | 447 |
| SEQ ID NO: 26 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI KRTVAAPSVF | 120 |
| light chain | IFPPSDEQLK SGTASVVCLL NNFYPREAKV QWKVDNALQS GNSQESVTEQ DSKDSTYSLS | 180 |
| | STLTLSKADY EKHKVYACEV THQGLSSPVT KSFNRGEC | 218 |
| SEQ ID NO: 27 | QVQLVQSGVE VKKPGASVKV SCKASGYTET NYYMYWVRQA PGQGLEWMGG INPSNGGTNF | 60 |
| pembrolizumab | NEKFKNRVTL TTDSSTTTAY MELKSLQFDD TAVYYCARRD YREDMGYDYW GQGTTVTVSS | 120 |
| variable heavy | | |
| chain | | |
| SEQ ID NO: 28 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI K | 111 |
| variable light | | |
| chain | | |
| SEQ ID NO: 29 | NYYMY | 5 |
| pembrolizumab | | |
| heavy chain | | |
| CDR1 | | |
| SEQ ID NO: 30 | GINPSNGGTN FNEKFK | 16 |
| pembrolizumab | | |
| heavy chain | | |
| CDR2 | | |
| SEQ ID NO: 31 | RDYREDMGED Y | 11 |
| pembrolizumab | | |
| heavy chain | | |
| CDR3 | | |
| SEQ ID NO: 32 | RASKGVSTSG YSYLH | 15 |
| pembrolizumab | | |
| light chain | | |
| CDR1 | | |
| SEQ ID NO: 33 | LASYLES | 7 |
| pembrolizumab | | |
| light chain | | |
| CDR2 | | |
| SEQ ID NO: 34 | QHSRDLPLT | 9 |
| pembrolizumab | | |
| light chain | | |
| CDR3 | | |
| SEQ ID NO: 35 | QVQLVQSGSE LKKPGASVKI SCKASGYTFT NYGMNWVRQA PGQGLQWMGW INTDSGESTY | 60 |
| pidilizumab | AEEFKGRFVF SLDTSVNTAY LQITSLTAED TGMYFCVRVG YDALDYWGQG TLVTVSSAST | 120 |
| heavy chain | KGPSVFPLAP SSKSTSGGTA ALGCLVKDYF PEPVTVSWNS GALTSGVHTF PAVLQSSGLY | 180 |
| | SLSSVVTVPS SSLGTQTYIC NVNHKPSNTK VDKRVEPKSC DKTHTCPPCP ARELLGGPSV | 240 |
| | FLFPPKPKDT LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSREEMTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID NO: 36 | EIVLTQSPSS LSASVGDRVT ITCSARSSVS YMHWFQQKPG KAPKLWIYRT SNLASGVPSR | 60 |
| pidilizumab | FSGSGSGTSY CLTINSLQPE DFATYYCQQR SSFPLTFGGG TKLEIKRTVA APSVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 37 | QVQLVQSGSE LKKPGASVKT SCKASGYTFT NYGMNWVRQA PGQGLQWMGW TNTDSGFSTY | 60 |
| pidilizumab | AEEFKGRFVF SLDTSVNTAY LQITSLTAED TGMYFCVRVG YDALDYWGQG TLVTVSS | 117 |
| variable heavy | | |
| chain | | |
| SEQ ID NO: 38 | EIVLTQSPSS LSASVGDRVT ITCSARSSVS YMHWFQQKPG KAPKLWIYRT SNLASGVPSR | 60 |
| pidilizumab | FSGSGSGTSY CLTINSLQPE DFATYYCQQR SSFPLTFGGG TKLEIK | 106 |
| variable light | | |
| chain | | |

The PD-L1 or PD-L2 inhibitor may be any PD-L or PD-L2 inhibitor or blocker known in the art. In particular, it is one of the PD-L1 or PD-L2 inhibitors or blockers described in more detail in the following paragraphs. The terms “inhibitor” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In some embodiments, the compositions and methods include a PD-L1 or PD-L2 inhibitor. In some embodiments, the PD-L1 and PD-L2 inhibitor is a small molecule. In some embodiments, the PD-L1 or PD-L2 inhibitor is an anti-PD-L1 or anti-PD-L2 antibody, a fragment thereof, including Fab fragments or single-chain variable fragments (scFv). In an aspect of the invention, the anti-PD-1 antibody or fragment thereof in any of the aforementioned embodiments is replaced by, or combined with, an anti-PD-L1 or anti-PD-L2 antibody or fragment thereof. In an embodiment, the antibody competes for binding with, and/or binds to an epitope on PD-L1 and/or PD-L2. In some embodiments, the PD-L1 or PD-L2 inhibitor is a monoclonal antibody. In some embodiments the PD-L1 or PD-L2 inhibitor is a polyclonal antibody. In some embodiments, a PD-L1 inhibitor is included in a composition or a method and is further combined with a BTK inhibitor. In some embodiments, an anti-PD-L1 monoclonal antibody is included in a composition or a method and is further combined with a BTK inhibitor. In some embodiments, a PD-L2 inhibitor is included in a composition or a method and is further combined with a BTK inhibitor. In some embodiments, an anti-PD-L2 monoclonal antibody is included in a composition or a method and is further combined with a BTK inhibitor. In some embodiments, a PD-L1 inhibitor is included in a composition or a method and is further combined with an antifolate compound. In some embodiments, an anti-PD-L1 monoclonal antibody is included in a composition or a method and is further combined with an antifolate compound. In some embodiments, a PD-L2 inhibitor is included in a composition or a method and is further combined with an antifolate compound. In some embodiments, an anti-PD-L2 monoclonal antibody is included in a composition or a method and is further combined with an antifolate compound.

In preferred embodiments, the compositions described herein provide a combination of a PD-L1 and/or PD-L2 inhibitor with a BTK inhibitor, or methods of using a combination of a PD-L1 and/or PD-L2 inhibitor with a BTK inhibitor. In some embodiments, the PD-L1 inhibitors provided herein are selective for PD-L1, in that the compounds bind or interact with PD-L1 at substantially lower concentrations than they bind or interact with other receptors, including the PD-L2 receptor. In certain embodiments, the compounds bind to the PD-L2 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L1 receptor.

Without being bound by any theory, it is believed that tumor cells express PD-L1, and that T cells express PD-1. However, PD-L1 expression by tumor cells is not required for efficacy of PD-1 or PD-L1 inhibitors or blockers. In an embodiment, the tumor cells express PD-L1. In another embodiment, the tumor cells do not express PD-L1. In some embodiments, the methods and compositions described herein include a combination of a PD-1 and a PD-L1 antibody, such as those described herein, in combination with a BTK inhibitor. The administration of a combination of a PD-1 and a PD-L1 antibody and a BTK inhibitor may be simultaneous or sequential.

In some embodiments, the compositions and methods described include a PD-L1 and/or PD-L2 inhibitor that binds human PD-L1 and/or PD-L2 with a K_{D} of about 100 pM or lower, binds human PD-L1 and/or PD-L2 with a K_{D} of about 90 pM or lower, binds human PD-L1 and/or PD-L2 with a K_{D} of about 80 pM or lower, binds human PD-L1 and/or PD-L2 with a K_{D} of about 70 pM or lower, binds human PD-L1 and/or PD-L2 with a K_{D} of about 60 pM or lower, a K_{D} of about 50 pM or lower, binds human PD-L1 and/or PD-L2 with a K_{D} of about 40 pM or lower, or binds human PD-L1 and/or PD-L2 with a KD of about 30 pM or lower,

In some embodiments, the compositions and methods described include a PD-L1 and/or PD-L2 inhibitor that binds to human PD-L1 and/or PD-L2 with a k_{assoc} of about 7.5×10⁵ l/M·s or faster, binds to human PD-L1 and/or PD-L2 with a k_{assoc} of about 8×10⁵ l/M·s or faster, binds to human PD-L1 and/or PD-L2 with a k_{assoc} of about 8.5×10⁵ l/M·s or faster, binds to human PD-L1 and/or PD-L2 with a k_{assoc} of about 9×10⁵ l/M·s or faster, binds to human PD-L1 and/or PD-L2 with a k_{assoc} of about 9.5×10⁵ l/M·s and/or faster, or binds to human PD-L1 and/or PD-L2 with a k_{assoc} of about 1×10⁶ l/M·s or faster.

In some embodiments, the compositions and methods described include a PD-L1 and/or PD-L2 inhibitor that binds to human PD-L1 or PD-L2 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower, binds to human PD-L1 or PD-L2 with a k_{dissoc} of about 2.7×10⁻⁵l/s or slower, or binds to human PD-L1 or PD-L2 with a k_{dissoc} of about 3×10⁻⁵ l/s or slower.

In some embodiments, the compositions and methods described include a PD-L1 and/or PD-L2 inhibitor that blocks or inhibits binding of human PD-L1 or human PD-L2 to human PD-1 with an IC₅₀ of about 10 nM or lower; blocks or inhibits binding of human PD-L1 or human PD-L2 to human PD-1 with an IC₅₀ of about 9 nM or lower; blocks or inhibits binding of human PD-L1 or human PD-L2 to human PD-1 with an IC₅₀ of about 8 nM or lower; blocks or inhibits binding of human PD-L or human PD-L2 to human PD-1 with an IC₅₀ of about 7 nM or lower; blocks or inhibits binding of human PD-L1 or human PD-L2 to human PD-1 with an IC₅₀ of about 6 nM or lower; blocks or inhibits binding of human PD-L1 or human PD-L2 to human PD-1 with an IC₅₀ of about 5 nM or lower; blocks or inhibits binding of human PD-L1 or human PD-L2 to human PD-1 with an IC₅₀ of about 4 nM or lower; blocks or inhibits binding of human PD-L1 or human PD-L2 to human PD-1 with an IC₅₀ of about 3 nM or lower; blocks or inhibits binding of human PD-L1 or human PD-L2 to human PD-1 with an IC₅₀ of about 2 nM or lower; or blocks human PD-1, or blocks binding of human PD-L1 or human PD-L2 to human PD-1 with an IC₅₀ of about 1 nM or lower.

In an embodiment, the anti-PD-L1 antibody is durvalumab, also known as MEDI4736 (which is commercially available from Medimmune, LLC, Gaithersburg, Md., a subsidiary of AstraZeneca plc.), or antigen-binding fragments, conjugates, or variants thereof. In an embodiment, the anti-PD-L1 antibody is an antibody disclosed in U.S. Pat. No. 8,779,108 or U.S. Patent Application Publication No. 2013/0034559, the disclosures of which are specifically incorporated by reference herein. The clinical efficacy of durvalumab (SEQ ID NO:403 and SEQ ID NO:404) has been described in: Page, et al., Ann. Rev. Med., 2014, 65, 185-202; Brahmer, et al., J. Clin. Oncol. 2014, 32, 5s (supplement, abstract 8021); and McDermott, et al., Cancer Treatment Rev., 2014, 40, 1056-64. The durvalumab monoclonal antibody includes a V_{H} region given by SEQ ID NO:41 (corresponding to SEQ ID NO:72 in U.S. Pat. No. 8,779,108) and a V_{L} region given by SEQ ID NO:42 (corresponding to SEQ ID NO:77 in U.S. Pat. No. 8,779,108). The durvalumab monoclonal antibody includes disulfide linkages at 22-96, 22″-96″, 23′-89′, 23′″-89′″, 135′-195′, 135′″-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In an embodiment, the anti-PD-L1 antibody is an immunoglobulin G1, anti-(human CD antigen CD274) (human monoclonal heavy chain), disulfide with human monoclonal i-chain, dimer. In an embodiment, the anti-PD-L1 antibody comprises the heavy and light chains of durvalumab (MEDI4736). In an embodiment, an anti-PD-L1 antibody comprises heavy and light chains having the sequences shown in SEQ ID NO:39 and SEQ ID NO:40, respectively, or antigen binding fragments, variants, or conjugates thereof. In an embodiment, an anti-PD-L1 antibody comprises heavy and light chains that are each at least 99% identical to the sequences shown in SEQ ID NO:39 and SEQ ID NO:40, respectively. In an embodiment, an anti-PD-L1 antibody comprises heavy and light chains that are each at least 98% identical to the sequences shown in SEQ ID NO:39 and SEQ ID NO:40, respectively. In an embodiment, an anti-PD-L1 antibody comprises heavy and light chains that are each at least 97% identical to the sequences shown in SEQ ID NO:39 and SEQ ID NO:40, respectively. In an embodiment, an anti-PD-L1 antibody comprises heavy and light chains that are each at least 96% identical to the sequences shown in SEQ ID NO:39 and SEQ ID NO:40, respectively. In an embodiment, an anti-PD-L1 antibody comprises heavy and light chains that are each at least 95% identical to the sequences shown in SEQ ID NO:39 and SEQ ID NO:40, respectively.

In an embodiment, the anti-PD-L1 antibody is an anti-PD-L1 biosimilar monoclonal antibody approved by drug regulatory authorities with reference to durvalumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

In an embodiment, the anti-PD-L1 antibody comprises V_{H} and V_{L} regions having the sequences shown in SEQ ID NO:41 (corresponding to SEQ ID NO:72 in U.S. Pat. No. 8,779,108) and SEQ ID NO:42 (corresponding to SEQ ID NO:77 in U.S. Pat. No. 8,779,108), respectively, as described in U.S. Pat. No. 8,779,108 or U.S. Patent Application Publication No. US 2013/0034559, the disclosures of which are specifically incorporated by reference herein, including antigen binding fragments, conjugates, and variants thereof. In an embodiment, an anti-PD-L1 antibody comprises V_{H} and V_{L} regions that are each at least 99% identical to the sequences shown in SEQ ID NO:41 and SEQ ID NO:42, respectively. In an embodiment, an anti-PD-L1 antibody comprises V_{H} and V_{L} regions that are each at least 98% identical to the sequences shown in SEQ ID NO:41 and SEQ ID NO:42, respectively. In an embodiment, an anti-PD-L1 antibody comprises V_{H} and V_{L} regions that are each at least 97% identical to the sequences shown in SEQ ID NO:41 and SEQ ID NO:42, respectively. In an embodiment, an anti-PD-L1 antibody comprises V_{H} and V_{L} regions that are each at least 96% identical to the sequences shown in SEQ ID NO:41 and SEQ ID NO:42, respectively. In an embodiment, an anti-PD-L1 antibody comprises V_{H} and V_{L} regions that are each at least 95% identical to the sequences shown in SEQ ID NO:41 and SEQ ID NO:42, respectively. In an embodiment, an anti-PD-L1 antibody comprises V_{H} and V_{L} regions that are each at least 90% identical to the sequences shown in SEQ ID NO:41 and SEQ ID NO:42, respectively.

In an embodiment, the anti-PD-L1 antibody comprises heavy chain CDR1, CDR2 and CDR3 domains having the sequences set forth in SEQ ID NO:43, SEQ ID NO:44, and SEQ ID NO:45, respectively, and light chain CDR1, CDR2 and CDR3 domains having the sequences set forth in SEQ ID NO:43, SEQ ID NO:44, and SEQ ID NO:45, respectively.

In an embodiment, an anti-PD-L1 antibody comprises a heavy chain that comprises CDR1, CDR2 and CDR3 domains that are each at least 95% identical to the sequences shown in SEQ ID NO:43, SEQ ID NO:44, and SEQ ID NO:45, respectively. In an embodiment, an anti-PD-L1 antibody comprises a heavy chain that comprises CDR1, CDR2 and CDR3 domains that are each at least 90% identical to the sequences shown in SEQ ID NO:43, SEQ ID NO:44, and SEQ ID NO:45, respectively. In an embodiment, an anti-PD-L1 antibody comprises a heavy chain that comprises CDR1, CDR2 and CDR3 domains that are each at least 85% identical to the sequences shown in SEQ ID NO:43, SEQ ID NO:44, and SEQ ID NO:45, respectively. In an embodiment, an anti-PD-L1 antibody comprises a heavy chain that comprises CDR1, CDR2 and CDR3 domains that are each at least 80% identical to the sequences shown in SEQ ID NO:43, SEQ ID NO:44, and SEQ ID NO:45, respectively. In another embodiment, the antibody competes for binding with, and/or binds to the same epitope on PD-L1 as the aforementioned antibodies.

In an embodiment, an anti-PD-L1 antibody comprises a light chain that comprises CDR1, CDR2 and CDR3 domains that are each at least 95% identical to the sequences shown in SEQ ID NO:46, SEQ ID NO:47, and SEQ ID NO:48, respectively. In an embodiment, an anti-PD-L1 antibody comprises a light chain that comprises CDR1, CDR2 and CDR3 domains that are each at least 90% identical to the sequences shown in SEQ ID NO:46, SEQ ID NO:47, and SEQ ID NO:48, respectively. In an embodiment, an anti-PD-L1 antibody comprises a light chain that comprises CDR1, CDR2 and CDR3 domains that are each at least 85% identical to the sequences shown in SEQ ID NO:46, SEQ ID NO:47, and SEQ ID NO:48, respectively. In an embodiment, an anti-PD-L1 antibody comprises a light chain that comprises CDR1, CDR2 and CDR3 domains that are each at least 80% identical to the sequences shown in SEQ ID NO:46, SEQ ID NO:47, and SEQ ID NO:48, respectively. In another embodiment, the antibody competes for binding with, and/or binds to the same epitope on PD-L1 as the aforementioned antibodies.

In an embodiment, anti-PD-L antibodies and other PD-L1 inhibitors include those described in U.S. Pat. No. 8,779,108 and U.S. Patent Application Publication No. US 2013/0034559A, the disclosures of which are hereby incorporated by reference. In another embodiment, antibodies that compete with any of these antibodies for binding to PD-L1 are also included.

In an embodiment, the anti-PD-L1 antibody is atezolizumab, also known as MPDL3280A or RG7446 (commercially available from Genentech, Inc., a subsidiary of Roche), or antigen-binding fragments, conjugates, or variants thereof. In an embodiment, the anti-PD-L1 antibody is an antibody disclosed in U.S. Pat. No. 8,217,149, the disclosure of which is specifically incorporated by reference herein. In an embodiment, the anti-PD-L1 antibody is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The atezolizumab monoclonal antibody includes a heavy chain given by SEQ ID NO:49 and a light chain given by SEQ ID NO:50. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88′″, and 134′″-194′″; intra-heavy-light chain disulfide linkages (h 5-CL 126) at 221-214′ and 221″-214′″; intra-heavy-heavy chain disulfide linkages (h 11, h 14) at 227-227″ and 230-230″; and N-glycosylation sites (H CH₂ N84.4>A) at 298 and 298′.

In an embodiment, the anti-PD-L1 antibody is an immunoglobulin G1 kappa, anti-(human PD-L1) humanized monoclonal antibody. In an embodiment, the anti-PD-L1 antibody comprises the heavy and light chains of atezolizumab (MPDL3280A). In an embodiment, an anti-PD-L1 antibody comprises heavy and light chains having the sequences shown in SEQ ID NO:49 and SEQ ID NO:50, respectively, or antigen binding fragments, variants, or conjugates thereof. In an embodiment, an anti-PD-L1 antibody comprises heavy and light chains that are each at least 99% identical to the sequences shown in SEQ ID NO:49 and SEQ ID NO:50, respectively. In an embodiment, an anti-PD-L1 antibody comprises heavy and light chains that are each at least 98% identical to the sequences shown in SEQ ID NO:49 and SEQ ID NO:50, respectively. In an embodiment, an anti-PD-L1 antibody comprises heavy and light chains that are each at least 97% identical to the sequences shown in SEQ ID NO:49 and SEQ ID NO:50, respectively. In an embodiment, an anti-PD-L1 antibody comprises heavy and light chains that are each at least 96% identical to the sequences shown in SEQ ID NO:49 and SEQ ID NO:50, respectively. In an embodiment, an anti-PD-L1 antibody comprises heavy and light chains that are each at least 95% identical to the sequences shown in SEQ ID NO:49 and SEQ ID NO:50, respectively.

In an embodiment, the anti-PD-L1 antibody is an anti-PD-L1 biosimilar monoclonal antibody approved by drug regulatory authorities with reference to atezolizumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

In an embodiment, the anti-PD-L1 antibody comprises the heavy and light chain CDRs or VRs of atezolizumab (MPDL3280A). In an embodiment, the anti-PD-L1 antibody V_{H} region comprises the sequence shown in SEQ ID NO:51 (corresponding to SEQ ID NO:20 in U.S. Pat. No. 8,217,149), and the anti-PD-L1 antibody V_{L} region comprises the sequence shown in SEQ ID NO:52 (corresponding to SEQ ID NO:21 in U.S. Pat. No. 8,217,149). In an embodiment, an anti-PD-L1 antibody comprises V_{H} and V_{L} regions that are each at least 99% identical to the sequences shown in SEQ ID NO:51 and SEQ ID NO:52, respectively. In an embodiment, an anti-PD-L1 antibody comprises V_{H} and V_{L} regions that are each at least 98% identical to the sequences shown in SEQ ID NO:51 and SEQ ID NO:52, respectively. In an embodiment, an anti-PD-L1 antibody comprises V_{H} and V_{L} regions that are each at least 97% identical to the sequences shown in SEQ ID NO:51 and SEQ ID NO:52, respectively. In an embodiment, an anti-PD-L1 antibody comprises V_{H} and V_{L} regions that are each at least 96% identical to the sequences shown in SEQ ID NO:51 and SEQ ID NO:52, respectively. In an embodiment, an anti-PD-L1 antibody comprises V_{H} and V_{L} regions that are each at least 95% identical to the sequences shown in SEQ ID NO:51 and SEQ ID NO:52, respectively.

In an embodiment, the anti-PD-L1 antibody comprises a heavy chain variable region (V_{H}) polypeptide that comprises a CDR1, CDR2, and CDR3 sequence, wherein the CDR1 sequence is given by SEQ ID NO:53 (GFTFSX₁SWIH) (corresponding to SEQ ID NO:1 in U.S. Pat. No. 8,217,149), the CDR2 sequence is SEQ ID NO:54 (AWIX₂PYGGSX₃YYADSVKG) (corresponding to SEQ ID NO:2 in U.S. Pat. No. 8,217,149), and the CDR3 sequence is SEQ ID NO:55 (RHWPGGFDY) (corresponding to SEQ ID NO:3 in U.S. Pat. No. 8,217,149), further wherein X₁ is D or G, X₂ is S or L, and X₃ is T or S, and the anti-PD-L1 antibody also comprises a light chain variable region (V_{L}) polypeptide that comprises a CDR1, CDR2, and CDR3 sequence wherein the CDR1 sequence is given by SEQ ID NO:56 (RASQX₄X5X₆TX₇X₈A) (corresponding to SEQ ID NO:8 in U.S. Pat. No. 8,217,149), the CDR2 sequence is given by SEQ ID NO:57 (SASX₉LX₁₀S) (corresponding to SEQ ID NO:9 in U.S. Pat. No. 8,217,149), and the CDR3 sequence is SEQ ID NO:58 (QQX₁₁X₁₂X₁₃X₁₄PX₁₅T) (corresponding to SEQ ID NO:10 in U.S. Pat. No. 8,217,149), further wherein further wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; and X₁₅ is A, W, R, P or T.

In an embodiment, the anti-PD-L1 antibody is avelumab, also known as MSB0010718C (commercially available from Merck KGaA/EMD Serono), or antigen-binding fragments, conjugates, or variants thereof. In an embodiment, the anti-PD-L1 antibody is an antibody disclosed in U.S. Patent Application Publication No. US 2014/0341917 A1, the disclosure of which is specifically incorporated by reference herein. The avelumab monoclonal antibody includes a heavy chain given by SEQ ID NO:59 and a light chain given by SEQ ID NO:60. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22′″-90′″, and 138′″-197′″; intra-heavy-light chain disulfide linkages (h 5-CL 126) at 223-215′ and 223″-215′″; intra-heavy-heavy chain disulfide linkages (h 11, h 14) at 229-229″ and 232-232″; N-glycosylation sites (H CH₂ N84.4) at 300, 300″; fucosylated complex bi-antennary CHO-type glycans; and H CHS K2 C-terminal lysine clipping at 450 and 450′.

In an embodiment, the anti-PD-L1 antibody is an immunoglobulin G1 lambda-1, anti-(human PD-L1) human monoclonal antibody. In an embodiment, the anti-PD-L1 antibody comprises the heavy and light chains of avelumab (MSB0010718C). In an embodiment, an anti-PD-L1 antibody comprises heavy and light chains having the sequences shown in SEQ ID NO:59 and SEQ ID NO:60, respectively, or antigen binding fragments, variants, or conjugates thereof. In an embodiment, an anti-PD-L1 antibody comprises heavy and light chains that are each at least 99% identical to the sequences shown in SEQ ID NO:59 and SEQ ID NO:60, respectively. In an embodiment, an anti-PD-L1 antibody comprises heavy and light chains that are each at least 98% identical to the sequences shown in SEQ ID NO:59 and SEQ ID NO:60, respectively. In an embodiment, an anti-PD-L1 antibody comprises heavy and light chains that are each at least 97% identical to the sequences shown in SEQ ID NO:59 and SEQ ID NO:60, respectively. In an embodiment, an anti-PD-L1 antibody comprises heavy and light chains that are each at least 96% identical to the sequences shown in SEQ ID NO:59 and SEQ ID NO:60, respectively. In an embodiment, an anti-PD-L1 antibody comprises heavy and light chains that are each at least 95% identical to the sequences shown in SEQ ID NO:59 and SEQ ID NO:60, respectively.

In an embodiment, the anti-PD-L1 antibody is an anti-PD-L1 biosimilar monoclonal antibody approved by drug regulatory authorities with reference to avelumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

In an embodiment, the anti-PD-L1 antibody V_{H} region comprises the sequence given in SEQ ID NO:61 (corresponding to SEQ ID NO:24 in U.S. Patent Application Publication No. US 2014/0341917 A1), and the anti-PD-L1 antibody V_{L} region comprises the sequence given in SEQ ID NO:62 (corresponding to SEQ ID NO:25 in U.S. Patent Application Publication No. US 2014/0341917 A1). In an embodiment, an anti-PD-L1 antibody comprises V_{H} and V_{L} regions that are each at least 99% identical to the sequences shown in SEQ ID NO:61 and SEQ ID NO:62, respectively. In an embodiment, an anti-PD-L1 antibody comprises V_{H} and V_{L} regions that are each at least 98% identical to the sequences shown in SEQ ID NO:61 and SEQ ID NO:62, respectively. In an embodiment, an anti-PD-L1 antibody comprises V_{H} and V_{L} regions that are each at least 97% identical to the sequences shown in SEQ ID NO:61 and SEQ ID NO:62, respectively. In an embodiment, an anti-PD-L1 antibody comprises V_{H} and V_{L} regions that are each at least 96% identical to the sequences shown in SEQ ID NO:61 and SEQ ID NO:62, respectively. In an embodiment, an anti-PD-L1 antibody comprises V_{H} and V_{L} regions that are each at least 95% identical to the sequences shown in SEQ ID NO:61 and SEQ ID NO:62, respectively.

In an embodiment, the anti-PD-L1 antibody comprises a heavy chain variable region (V_{H}) polypeptide that comprises a CDR1, CDR2, and CDR3 sequence, wherein the CDR1 sequence is given by SEQ ID NO:63 (corresponding to SEQ ID NO:15 in U.S. Patent Application Publication No. US 2014/0341917 A1), the CDR2 sequence is given by SEQ ID NO:64 (corresponding to SEQ ID NO:16 in U.S. Patent Application Publication No. US 2014/0341917 A1), and the CDR3 sequence is given by SEQ ID NO:65 (corresponding to SEQ ID NO:17 in U.S. Patent Application Publication No. US 2014/0341917 A1), and the anti-PD-L1 antibody also comprises a light chain variable region (V_{L}) polypeptide that comprises a CDR1, CDR2, and CDR3 sequence wherein the CDR1 sequence is given by SEQ ID NO:66 (corresponding to SEQ ID NO:18 in U.S. Patent Application Publication No. US 2014/0341917 A1), the CDR2 sequence is given by SEQ ID NO:67 (corresponding to SEQ ID NO:19 in U.S. Patent Application Publication No. US 2014/0341917 A1), and the CDR3 sequence is given by SEQ ID NO:68 (corresponding to SEQ ID NO:20 in U.S. Patent Application Publication No. US 2014/0341917 A1).

In an embodiment, an anti-PD-L1 antibody comprises a heavy chain that comprises CDR1, CDR2 and CDR3 domains that are each at least 95% identical to the sequences shown in SEQ ID NO:63, SEQ ID NO:64, and SEQ ID NO:65, respectively. In an embodiment, an anti-PD-L1 antibody comprises a heavy chain that comprises CDR1, CDR2 and CDR3 domains that are each at least 90% identical to the sequences shown in SEQ ID NO:63, SEQ ID NO:64, and SEQ ID NO:65, respectively. In an embodiment, an anti-PD-L1 antibody comprises a heavy chain that comprises CDR1, CDR2 and CDR3 domains that are each at least 85% identical to the sequences shown in SEQ ID NO:63, SEQ ID NO:64, and SEQ ID NO:65, respectively. In an embodiment, an anti-PD-L1 antibody comprises a heavy chain that comprises CDR1, CDR2 and CDR3 domains that are each at least 80% identical to the sequences shown in SEQ ID NO:63, SEQ ID NO:64, and SEQ ID NO:65, respectively. In another embodiment, the antibody competes for binding with, and/or binds to the same epitope on PD-L1 as the aforementioned antibodies.

In an embodiment, an anti-PD-L1 antibody comprises a light chain that comprises CDR1, CDR2 and CDR3 domains that are each at least 95% identical to the sequences shown in SEQ ID NO:66, SEQ ID NO:67, and SEQ ID NO:68, respectively. In an embodiment, an anti-PD-L1 antibody comprises a light chain that comprises CDR1, CDR2 and CDR3 domains that are each at least 90% identical to the sequences shown in SEQ ID NO:66, SEQ ID NO:67, and SEQ ID NO:68, respectively. In an embodiment, an anti-PD-L1 antibody comprises a light chain that comprises CDR1, CDR2 and CDR3 domains that are each at least 85% identical to the sequences shown in SEQ ID NO:66, SEQ ID NO:67, and SEQ ID NO:68, respectively. In an embodiment, an anti-PD-L1 antibody comprises a light chain that comprises CDR1, CDR2 and CDR3 domains that are each at least 80% identical to the sequences shown in SEQ ID NO:66, SEQ ID NO:67, and SEQ ID NO:68, respectively. In another embodiment, the antibody competes for binding with, and/or binds to the same epitope on PD-L1 as the aforementioned antibodies.

In an embodiment, anti-PD-L1 antibodies and other PD-L1 inhibitors include those described in U.S. Patent Application Publication No. US 2014/0341917 A1, the disclosure of which is hereby incorporated by reference. In another embodiment, antibodies that compete with any of these antibodies for binding to PD-L1 are also included.

In an embodiment, the anti-PD-L1 antibody is MDX-1105, also known as BMS-935559, which is disclosed in U.S. Pat. No. 7,943,743 B2, the disclosures of which are specifically incorporated by reference herein. In an embodiment, the anti-PD-L1 antibody is selected from the anti-PD-L1 antibodies disclosed in U.S. Pat. No. 7,943,743 B2, which are specifically incorporated by reference herein.

In an embodiment, the anti-PD-L1 antibody is a commercially-available monoclonal antibody, such as INVIVOMAB anti-m-PD-L1 clone 10F.9G2 (Catalog # BE0101, Bio X Cell, Inc., West Lebanon, N.H., USA). In an embodiment, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

In an embodiment, the anti-PD-L2 antibody is a commercially-available monoclonal antibody, such as BIOLEGEND 24F. 10C12 Mouse IgG2a, κ isotype (catalog #329602 Biolegend, Inc., San Diego, Calif.), SIGMA anti-PD-L2 antibody (catalog # SAB3500395, Sigma-Aldrich Co., St. Louis, Mo.), or other commercially-available anti-PD-L2 antibodies known to one of ordinary skill in the art.

Monoclonal antibodies that inhibit PD-L1 and/or PD-L2 can be prepared by procedures known to those of ordinary knowledge and skill in the art, e.g., by injecting test subjects with PD-L1 or PD-L2 antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, myeloma cells, or other suitable cells that do not otherwise produce immunoglobulin protein, to obtain the synthesis of monoclonal antibodies in the recombinant host cells. The details of recombinant production of specific antibodies may be found in the references cited in the foregoing, the disclosures of which are incorporated by reference herein. Monoclonal antibodies that inhibit PD-1 can be prepared by standard molecular biology methods using the sequences provided herein by reverse translation and insertion into appropriate DNA or RNA vectors.

The anti-PD-L1 antibody sequences referenced in some of the foregoing embodiments are summarized in Table 3.

TABLE 3
Anti-PD-L1 antibody amino acid sequences.

| Identifier | Sequence (One-Letter Amino Acid Symbols) | |
|---|---|---|
| SEQ ID NO: 39 | EVQLVESGGG LVQPGGSLRL SCAASGFTFS RYWMSWVRQA PGKGLEWVAN IKQDGSEKYY | 60 |
| durvalumab | VDSVKGRFTI SRDNAENSLY LQMNSLEAED TAVYYCAREG GWFGELAFDY WGQGTLVTVS | 120 |
| (MEDI4736) | SASTKGPSVF ELAPSSKSTS GGTAALGCLV KDYFPEPVTV SWNSGALTSG VHTFEAVLQS | 180 |
| heavy chain | SGLYSLSSVV TVMSSSLGTQ TYICNVNHKP SNTKVDKRVE PKSCDKTHTC PPCPAPEFEG | 240 |
| | GPSVFLFPPK PIOTLMISRT PEVTCVVVDV SHEDPEVKFN WYVDGVEVHN AKTKPREEQY | 300 |
| | NSTYRVVSVL TVLHQDWLNG KEYKCKVSNK ALPASTEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 40 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIE DRFSGSGSGT | 120 |
| (MEDI4736) | DFTLTISRLE PEDFAVYYCQ QYGSLPWTFG QGTKVEIKRT VAAPSVFIFP PSDEQLKSGT | 180 |
| light chain | ASVVCLLNNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 41 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| variable | S | 121 |
| heavy chain | | |
| SEQ ID NO: 42 | EIVLTQSPGT LSLSPGERAT LSCRASQRVS SSYLAWYQQK PGQAPRLLIY DASSRATGIP | 60 |
| durvalumab | DRFSGSGSGT DFTLTISRLR PEDFAVYYCQ QYGSLPWTFG QGTKVEIK | 108 |
| variable | | |
| light chain | | |
| SEQ ID NO: 43 | RYWMS | 5 |
| durvalumab | | |
| heavy chain | | |
| CDR1 | | |
| SEQ ID NO: 44 | NIKQDGSEKY YVDSVKG | 17 |
| durvalumab | | |
| heavy chain | | |
| CDR2 | | |
| SEQ ID NO: 45 | EGGWFGELAF DY | 12 |
| durvalumab | | |
| heavy chain | | |
| CDR3 | | |
| SEQ ID NO: 46 | RASQRVSSSY LA | 12 |
| durvalumab | | |
| light chain | | |
| CDR1 | | |
| SEQ ID NO: 47 | DASSRAT | 7 |
| durvalumab | | |
| light chain | | |
| CDR2 | | |
| SEQ ID NO: 43 | QQYGSLPWT | 9 |
| durvalumab | | |
| light chain | | |
| CDR3 | | |
| SEQ ID NO: 49 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSSAS | 120 |
| (MPDL3280A) | TKGPSVFPLA PSSKSTSGGT AALGCLVKDY FPEPVTVSWN SGALTSGVHT FPAVLOSSGL | 180 |
| heavy chain | YSLSSVVTVP SSSLGTQTYI CNVNHKESNT KVDKKVEPKS CDKTHTCPPC PAPELLGGPS | 240 |
| | VELFPEKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPCK | 448 |
| SEQ ID NO: 50 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKRTV AAPSVFIFPP | 120 |
| (MPDL3280A) | SDEQLKSGTA SVVCLLNNFY PREAKVQWKV DNALQSGNSQ ESVTEQDSKD STYSLSSTLT | 180 |
| light chain | LSKADYEKHK VYACEVTHQG LSSPVTKSFN RGEC | 214 |
| SEQ ID NO: 51 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSA | 118 |
| variable | | |
| heavy chain | | |
| SEQ ID NO: 52 | DIOMTOSESS LSASVGDRVT ITCRASQDVS TAVAWYQQKP GKAPKLLIYS ASFLYSGVPS | 60 |
| atezoliznmab | RFSGSGSGTD FTLTISSLQP EDFATYYCQQ YLYHPATFGQ GTKVEIKR | 108 |
| variable | | |
| light chain | | |
| SEQ ID NO: 53 | GFTFSXSWIH | 10 |
| atezolizumab | | |
| heavy chain | | |
| CDR1 | | |
| SEQ ID NO: 54 | AWIXPYGGSX YYADSVKG | 18 |
| atezolizumab | | |
| heavy chain | | |
| CDR2 | | |
| SEQ ID NO: 55 | RHWPGGFDY | 9 |
| atezolizumab | | |
| heavy chain | | |
| CDR3 | | |
| SEQ ID NO: 56 | RASQXXXTXX A | 11 |
| atezolizumab | | |
| light chain | | |
| CDR1 | | |
| SEQ ID NO: 57 | SASXLXS | 7 |
| atezolizumab | | |
| light chain | | |
| CDR2 | | |
| SEQ ID NO: 58 | QQXXXXPXT | 9 |
| atezolizumab | | |
| light chain | | |
| CDR3 | | |
| SEQ ID NO: 59 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| (MSB0010718C) | ASTKGPSVFP LAPSSKSTSG GTAALGCLVK DYFPEPVTVS WNSGALTSGV HTFPAVLQSS | 180 |
| heavy chain | GLYSLSSVVT VPSSSLGTQT YICNVNHKPS NTKVDKKVEP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVESCSV MHEALHNHYT QKSLSLSPGK | 450 |
| SEQ ID NO: 60 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL GQPKANPTVT | 120 |
| (MSB0010718C) | LFPPSSEELQ ANKATLVCLI SDFYPGAVTV AWKADGSPVK AGVETTKPSK QSNNKYAASS | 180 |
| light chain | YLSLTPEQWK SHRSYSCQVT HEGSTVEKTV APTECS | 216 |
| SEQ ID NO: 61 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| variable | | |
| heavy chain | | |
| SEQ ID NO: 62 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL | 110 |
| variable | | |
| light chain | | |
| SEQ ID NO: 63 | SYIMM | 5 |
| avelumab | | |
| heavy chain | | |
| CDR1 | | |
| SEQ ID NO: 64 | SIYPSGGITF YADTVKG | 17 |
| avelumab | | |
| heavy chain | | |
| CDR2 | | |
| SEQ ID NO: 65 | IKLGTVTTVD Y | 11 |
| avelumab | | |
| heavy chain | | |
| CDR3 | | |
| SEQ ID NO: 66 | TGTSSDVGGY NYVS | 14 |
| avelumab | | |
| light chain | | |
| CDR1 | | |
| SEQ ID NO: 67 | DVSNRPS | 7 |
| avelumab | | |
| light chain | | |
| CDR2 | | |
| SEQ ID NO: 68 | SSYTSSSTRV | 10 |
| avelumab | | |
| light chain | | |
| CDR3 | | |

The preparation, properties, and uses of suitable PD-1 and PD-L1 inhibitors are described in, e.g., U.S. Pat. No. 8,008,449 or U.S. Patent Application Publication Nos. 2009/0217401 A1 or 2013/0133091 A1; U.S. Pat. No. 8,354,509 and U.S. Patent Application Publication Nos. US 2010/0266617 A1, US 2013/0108651 A1, and US 2013/0109843 A2; U.S. Pat. Nos. 8,287,856, 8,580,247, and 8,168,757 and U.S. Patent Application Publication Nos. US 2009/0028857 A1, US 2010/0285013 A1, US 2013/0022600 A1, and US 2011/0008369 A1; U.S. Pat. No. 8,779,108 or U.S. Patent Application Publication No. US 2013/0034559 A1; U.S. Pat. No. 8,217,149 and U.S. Patent Application Publication Nos. US 2010/0203056 A1, US 2013/0045200 A1, US 2013/0045201 A1, US 2013/0045202 A1, or US 2014/0065135 A1; and U.S. Patent Application Publication No. US 2014/0341917 A1, the disclosures of each of which are incorporated by reference herein.

In any of the foregoing embodiments, the PD-1 and/or PD-L1 inhibitors or combinations thereof may be administered before, concurrently, or after administration of the antifolate compounds and the BTK inhibitors.

In an embodiment, the PD-1 or PD-L1 inhibitor is an anti-PD-1 or anti-PD-L1 biosimilar monoclonal antibody approved by drug regulatory authorities with reference to inhibitor selected from the group consisting of nivolumab, pembrolizumab, pidilizumab, durvalumab, atezolizumab, or avclumab. In an embodiment, the biosimilar comprises an anti-PD-1 or anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab, pembrolizumab, pidilizumab, durvalumab, atezolizumab, or avelumab. In some embodiments, the one or more post-translational modifications are selected from one or more of: glycosylation, oxidation, deamidation, and truncation. In some embodiments, the biosimilar is an anti-PD1 or anti-PD-L1 antibody authorized or submitted for authorization, wherein the anti-PD1 or anti-PD-L1 antibody is provided in a formulation which differs from the formulations of a reference medicinal product or reference biological product, wherein the reference medicinal product or reference biological product is nivolumab, pembrolizumab, pidilizumab, durvalumab, atezolizumab, or avelumab. The anti-PD1 or anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab, pembrolizumab, pidilizumab, durvalumab, atezolizumab, or avelumab. In some embodiments, the biosimilar comprises one or more excipients selected from tris-hydrochloride, sodium chloride, mannitol, pentetic acid, polysorbate 80, sodium hydroxide, and hydrochloric acid.

In an embodiment, the invention provides a method of treating a cancer or an immune, autoimmune, or inflammatory disease in a human comprising the step of administering to said human a BTK inhibitor, or a pharmaceutically acceptable salt or ester, prodrug, cocrystal, solvate or hydrate thereof, and antifolate compounds, including an antibody, antibody fragment, derivative, conjugate, variant, radioisotope-labeled complex, and biosimilar thereof, and further comprising the step of administering an PD-1 or PD-L1 inhibitor, or an antigen-binding fragment, derivative, conjugate, variant, radioisotope-labeled complex, or biosimilar thereof. In an embodiment, the BTK inhibitor is a compound selected from the group consisting of Formula (2), Formula (10), and Formula (21); the antifolate compound is a compound selected from the group consisting of methotrexate, pemetrexed, raltitrexed and pharmaceutically acceptable salts, solvates, hydrates, cocrystals, prodrugs, and combinations thereof; and the PD-1 or PD-L1 inhibitor is selected from the group consisting of nivolumab, pembrolizumab, pidilizumab, durvalumab, atezolizumab, avelumab, and antigen-binding fragments, variants, conjugates, or biosimilars thereof.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Synergistic Combinations of BTK Inhibitors and Antifolates

The in vivo use of the BTK inhibitor of Formula (2) and methotrexate (“MTX”) was tested in a mouse model to determine the combination's effectiveness to inhibit inflammation, cartilage destruction, pannus formation and bone resorption associated with type II collagen arthritis. DBA/1lacJ Mice mice were anesthetized with Isoflurane and given intradermal injections of a total of 100 μL of 2 mg/mL of Type II collagen in 2.5 mg/mL Freund's complete adjuvant at the base of the tail on study days zero and 21. MTX treatments were initiated on day 18 and the BTK inhibitor of Formula (2) treatments were initiated on day 28 of the vehicle treated animals (arthritis score between 0.5 and 1) and continued for the remainder of the study. The BTK inhibitor of Formula (2) vehicle treatments were dosed 6 hours post MTX dosing.

Formula (2) and MTX were formulated with 0.5% w/v Methylcellulose; 4000 cps (DOW, Methocel A4M or Equivalent); 0.1% v/v Polysorbate 80 (Tween 80) and DI or RO water. Formula (2) was formulated at concentrations of 0.1 mg/mL and 0.5 mg/mL and MTX was formulated at 0.03 mg/mL, 0.05 mg/mL, 0.1 mg/ml and 0.5 mg/mL. MTX was administered PO gavage at doses of QD 0.3 mg/kg and QD 0.5 mg/kg in the mornings. Six hours later, Formula (2) was administered PO gavage at doses of QD 1 mg/kg and QD 5 mg/kg. All mice survived through study termination.

Daily clinical scores were based on daily paw scores, an area under the curve (AUC) calculation of the paw scoring over time, and the hind paw, ankle, and optionally knees histopathology, body weight measurements and body weight change from the start of dosing will be evaluated for signs of toxic effects of the test article(s) and/or vehicle(s). The daily clinical scores were determined for each paw on study days 18-43. The scoring was based on the following criteria: 0=normal; 1=one hind or fore paw joint affected or minimal diffuse erythema and swelling; 2=two hind or fore paw joints affected or mild diffuse erythema and swelling; 3=three hind or fore paw joints affected or moderate diffuse erythema and swelling; 4=marked diffuse erythema and swelling, or four digit joints affected; and 5=severe diffuse erythema and severe swelling entire paw, unable to flex digits.

Clinical scores were given for each of the paws (right front, left front, right rear, left rear) on Study Days 18-43. Clinical data for paw scores (means for animal) were then analyzed by determining the area under the dosing curve (AUC). AUC was calculated from MTX dosing initiation (Day 18) through study termination (Day 43) and from the BTK inhibitor of Formula (2) dosing initiation (Day 28) through study termination. For calculation of AUC, the daily mean scores for each mouse were entered into Microsoft Excel and the area between the treatment days and the final day was computed. Means for each group were determined. A one-way analysis of variance (1-way ANOVA) along with a Dunnett's or Sidak's post-hoc analysis or a Kruskal-Wallis test (non-parametric) along with a Dunn's posthoc analysis was used to the evaluate data collected in this study. A Student's two-tailed t-test was used to compare normal versus disease controls for model validation. Unless indicated, Bolder BioPATH, Inc. performs statistical analysis on raw (untransformed) data only. Statistical tests make certain assumptions regarding normality and homogeneity of variance, and further analysis may be required if testing resulted in violations of these assumptions. P values were rounded to three decimal places. Significance for all tests was set at p<0.050. Statistical analysis was performed using Prism 6.0d softwareGraphPad. Percent inhibition is calculated using the following formula:

% Change=B/A×100A=Mean Normal−Mean Disease Control where B=Mean Treated−Mean Disease Control

On study day 43, mice were euthanized and necropsy specimens were obtained. After terminal bleeds, animals were euthanized by cervical dislocation. Fore paws, hind paws, and knees were harvested and placed in 10% neutral buffered formalin (NBF) for microscopy. Spleens were harvested from a subset of mice and processed for splenocytes.

After 1-2 days in fixative and 4-5 days in 5% formic acid for decalcification, tissues were trimmed, and processed for paraffin embedding. Paws were embedded in paraffin in the frontal plane and knees were embedded with the patella facing down. Ankles, if left attached to the hind paw, were also embedded in the frontal plane but in some instances were detached and sectioned in the sagittal plane for special purposes. Left/right pairs were typically embedded in the same block. Sections were then cut and stained with toluidine blue.

When scoring paws or ankles from mice with lesions of type II collagen arthritis, severity of changes as well as number of individual joints affected was considered. When only one to three joints of the paws or ankles out of a possibility of numerous metacarpal/metatarsal/digit or tarsal/tibiotarsal joints was affected, an arbitrary assignment of a maximum score of 0.5, 1, 2 or 3 for parameters below was given depending on severity of changes. If more than three joints were involved, the criteria below were applied to the most severely affected/majority of joints. In the case of knees, severity of changes in medial and lateral, as well as femoropatellar spaces, were considered.

The following parameters were scored according to the indicated criteria: Inflammation Score, Pannus Score, Cartilage Damage Score, Bone Resorption Score, and Periosteal New Bone Formation Score and Measurements. Unless otherwise indicated, criteria apply to both paws and knees. Mean values for each parameter are determined separately for the paws, knees, and the entire animal (if applicable).

The Inflammation Score was determined based on the following criteria for Paw Score and Knee Score.

The Paw Score was classified based on the following criteria: 0=Normal; 0.5=Very minimal, affects only 1 joint or minimal multifocal periarticular infiltration of inflammatory cells; 1=Minimal infiltration of inflammatory cells in synovium and periarticular tissue of affected joints; 2=Mild infiltration of inflammatory cells. If referring to paws, generally restricted to affected joints (1-3 affected); 3=Moderate infiltration with moderate edema. If referring to paws, restricted to affected joints, generally 3-4 joints and the wrist or ankle; 4=Marked infiltration affecting most areas with marked edema, 1 or 2 unaffected joints may be present; 5=Severe diffuse infiltration with severe edema affecting all joints (to some extent) and periarticular tissues.

The Knee Score was classified based on the following criteria: 0=Normal; 0.5=Very minimal, affects only one area of the synovium or minimal multifocal periarticular infiltration of inflammatory cells; 1=Minimal infiltration of inflammatory cells in synovium and periarticular tissue of affected synovial areas; 2=Mild diffuse infiltration of inflammatory cells; 3=Moderate diffuse infiltration of inflammatory cells; 4=Marked diffuse infiltration of inflammatory cells; 5=Severe diffuse infiltration of inflammatory cells.

The inflammatory infiltrate in mice and rats with type II collagen arthritis consists of neutrophils and macrophages with smaller numbers of lymphocytes when the lesions are in the acute to subacute phase. Tissue edema and neutrophil exudates within the joint space are common in the acute to subacute phase. As the inflammation progresses to chronic, mononuclear inflammatory cells (monocytes, lymphocytes) predominate and fibroblast proliferation, often with deposition of metachromatic matrix, occurs in synovium and periarticular tissue. Exudate is less common in the joint space. Unless indicated in the comments area, the inflammation type is acute to subacute.

DBA mice have an increased incidence of dactylitis and onchyoperiostitis affecting the nail bed and distal phalynx as reported in Lories, et al., Ann. Rheum. Dis. 2004, 63, 595-598. These lesions are recorded but were not included in the inflammation score.

The Pannus Score was based on the following criteria: 0=Normal; 0.5=Very minimal, If paws, affects only one joint at marginal zone; 1=Minimal infiltration of pannus in cartilage and subchondral bone, marginal zones. If paws, affects two or more joints; 2=Mild infiltration with marginal zone destruction of hard tissue in affected joints; 3=Moderate infiltration with moderate hard tissue destruction in affected joints; 4=Marked infiltration with marked destruction of joint architecture, affecting most joints; 5=Severe infiltration associated with total or near total destruction of joint architecture, affects all joints.

The Cartilage Damage Score was based on the following criteria: 0=Normal; 0.5=Very minimal=Affects marginal zones only of one to several areas (knees) or joints (paws); 1=Minimal=Generally minimal to mild loss of toluidine blue staining (proteoglycan) with no obvious chondrocyte loss or collagen disruption in affected joints/areas; 2=Mild=Generally mild loss of toluidine blue staining (proteoglycan) with focal areas of chondrocyte loss and/or collagen disruption in some affected joints/areas. Paws may have one or two digit joints with near total to total loss of cartilage; 3=Moderate=Generally moderate loss of toluidine blue staining (proteoglycan) with multifocal chondrocyte loss and/or collagen disruption in affected joints/areas. Paws may have three or four joints with near total or total loss. In the knee, some matrix remains on any affected surface with areas of severe matrix loss; 4=Marked=Marked loss of toluidine blue staining (proteoglycan) with multifocal marked (depth to deep zone or tidemark) chondrocyte loss and/or collagen disruption in most joints with a few unaffected or mildly affected. In the knee, one surface with total to near total cartilage loss; 5=Severe=Severe diffuse loss of toluidine blue staining (proteoglycan) with severe (depth to tide mark) chondrocyte loss and/or collagen disruption in most or all joints. In the knee, two or more surfaces with total to near total cartilage loss.

The Bone Resorption Score was determined based on the following criteria for Paw Score and Knee score.

The Paw Score was based on the following criteria: 0=Normal; 0.5=Very Minimal=Affects only 1 joint or is restricted to cortical/subperiosteal areas; I=Minimal=Small/few areas of definite resorption, not readily apparent on low magnification, rare osteoclasts in affected joints, restricted to marginal zones; 2=Mild=More numerous/larger areas of resorption, osteoclasts more numerous in affected joints, mainly in marginal zones but some extension to load bearing areas, may have endosteal proliferation in areas of resorption; 3=Moderate=Obvious resorption of medullary trabecular and cortical bone without widespread full thickness defects in cortex, loss of medullary trabeculae, lesion apparent on low magnification, osteoclasts more numerous in affected joints, may have endosteal proliferation in areas of resorption; 4=Marked=Full thickness defects in cortical bone, often with distortion of profile of remaining cortical surface, marked loss of medullary bone, numerous osteoclasts, affects most joints, may have endosteal proliferation in areas of resorption; 5=Severe=Full thickness defects in cortical bone and destruction of joint architecture of all joints, may have endosteal proliferation in areas of resorption.

The Knee Score was based on the following criteria: 0=Normal; 0.5=Very Minimal=Minimal resorption affects only marginal zones; I=Minimal=Small areas of resorption, not readily apparent on low magnification, approximately 1-10% of total joint width of subchondral bone affected; 2=Mild=More numerous areas of resorption, definite loss of subchondral bone, approximately 11-25% of total joint width of subchondral bone affected; 3=Moderate=Obvious resorption of subchondral bone, approximately 26-50% of total joint width of subchondral bone affected; 4=Marked=Obvious resorption of subchondral bone, approximately 51-75% of total joint width of subchondral bone affected; 5=Severe=Distortion of entire joint due to destruction, approximately 76-100% of total joint width of subchondral bone affected.

Periosteal new bone formation score and measurements were also assessed. Studies that go beyond the acute inflammatory stage often show varying degrees of periosteal new bone formation. The width of the largest area of new bone formation in a non-tangential section (“Periosteal New Bone Formation Score”) is measured and used to determine a score based on the following criteria for Paw Score and Knee Score.

The Paw Score was based on the following criteria: 0=Normal, no periosteal proliferation; 0.5=Minimal focal or multifocal early proliferation, measures less than 40 μm width (<1 unit on 25×); 1=Minimal multifocal early proliferation, measures 40-80 μm width (1-2 units on 25×); 2=Mild multifocal to diffuse with widths that measure approximately 120-200 μm (3-5 units on 25×); 3=Moderate diffuse with widths that measure 240-280 μm (6-7 units on 25×); 4=Marked diffuse with widths that measure 320-400 μm (8-10 units on 25×); 5=Severe, diffuse with widths that measure greater than 400 μm (>10 units on 25×).

The Knee Score was based on the following criteria: 0=Normal, no periosteal proliferation; 0.5=Minimal focal or multifocal early proliferation, measures 40 μm width or less (1-2 units on 50×); 1=Minimal multifocal early proliferation, measures approximately 40-80 μm width (3-4 units on 50×); 2=Mild multifocal to diffuse with widths that measures approximately 100-140 μm (5-7 units on 50×); 3=Moderate diffuse with widths that measure approximately 160-220 μm (8-11 units on 50×); 4=Marked diffuse with widths that measure approximately 240-300 μm (12-15 units on 50×); 5=Severe, diffuse with widths that measure greater than 300 μm (>15 units on 50×).

A sum of the five histopathology scores was also calculated for each joint.

Live phase and necropsy parameters were determined for various clinical and histopathology data including (i) change in body weight during days 18-43; (ii) clinical arthritis score AUC during days 18-43; (iii) clinical arthritis score AUC during days 28-43; (iv) percent incidence; and (v) histopathology summed scores for all joints. The data is summarized in Table 4. A detailed discussion of each data set is given below.

TABLE 4
Summary of Clinical and Histopathology Data

| | | Change in | Clinical | Clinical | | |
|---|---|---|---|---|---|---|
| | | Body | Arthritis | Arthritis | | Histopathology |
| | | Weight (g) | Score AUC | Score AUC | Percent | Summed Scores |
| Group | Treatment | Day 18-43 | Day 18-43 | Day 28-43 | Incidence | (All Joints) |
| 1 | Normal + | †0.67 | †0.00 | †0.00 | 0% | †0.04 (0.04) |
| | Vehicle, PO, | (0.20) | (0.00) | (0.00) | | |
| | QD | | | | | |
| 2 | MTX Vehicle | −0.94 | 71.73 | 65.70 | 100% | 15.02 (1.32) |
| | PO, QD (d18-43) + | (0.26) | (4.38) | (3.73) | | |
| | BTK | | | | | |
| | inhibitor of | | | | | |
| | formula (2) | | | | | |
| | Vehicle PO, QD | | | | | |
| | (d28-43) | | | | | |
| 3 | MTX Vehicle | −0.19 | 61.41 | 54.81 | 100% | 11.03 (1.01) |
| | PO, QD (d18-43) + | (0.18) | (3.58) | (3.58) | | |
| | BTK | | | | | |
| | inhibitor of | | | | | |
| | formula (2) (1 mg/kg) | | | | | |
| | PO, QD | | | | | |
| | (d28-43) | | | | | |
| 4 | MTX Vehicle | *0.12 | *27.30 | *21.27 | 42% | *4.15 (1.01) |
| | PO, QD (d18-43) + | (0.28) | (6.04) | (5.39) | | |
| | BTK | | | | | |
| | inhibitor of | | | | | |
| | formula (2) (5 mg/kg) | | | | | |
| | PO, QD | | | | | |
| | (d28-43) | | | | | |
| 5 | MTX (0.3 mg/kg) | −1.16 | 51.72 | 48.82 | 100% | 10.24 (1.66) |
| | PO, QD | (0.48) | (5.38) | (4.96) | | |
| | (d18-43) + BTK | | | | | |
| | inhibitor of | | | | | |
| | formula (2) | | | | | |
| | Vehicle PO, QD | | | | | |
| | (d28-43) | | | | | |
| 6 | MTX (0.5 mg/kg), | −0.83 | 46.79 | 45.51 | 100% | 8.46 (1.36) |
| | PO, QD | (0.19) | (5.73) | (5.56) | | |
| | (d18-43) + BTK | | | | | |
| | inhibitor of | | | | | |
| | formula (2) | | | | | |
| | Vehicle PO, QD | | | | | |
| | (d28-43) | | | | | |
| 7 | MTX (0.3 mg/kg) | −0.17 | *‡33.82 | *30.84 | 83% | *5.37 (1.35) |
| | PO, QD | (0.21) | (6.59) | (5.88) | | |
| | (d18-43) + BTK | | | | | |
| | inhibitor of | | | | | |
| | formula (2) (1 mg/kg) | | | | | |
| | PO, QD | | | | | |
| | (d28-43) | | | | | |
| 8 | MTX (0.5 mg/kg) | −0.31 | *‡29.04 | *‡27.59 | 75% | *‡3.88 (1.16) |
| | PO, QD | (0.30) | (6.24) | (5.95) | | |
| | (d18-43) + BTK | | | | | |
| | inhibitor of | | | | | |
| | formula (2) (1 mg/kg) | | | | | |
| | PO, QD | | | | | |
| | (d28-43) | | | | | |
| 9 | MTX (0.3 mg/kg) | −0.39 | *§15.54 | *§12.61 | 42% | *§1.39 (0.40) |
| | PO, QD | (0.16) | (3.26) | (3.00) | | |
| | (d18-43) + BTK | | | | | |
| | inhibitor of | | | | | |
| | formula (2) (5 mg/kg) | | | | | |
| | PO, QD | | | | | |
| | (d28-43) | | | | | |
| 10 | MTX (0.5 mg/kg) | −0.21 | *§9.74 | *§8.16 | 17% | *§1.77 (0.68) |
| | PO, QD | (0.16) | (3.61) | (3.22) | | |
| | (d18-43) + BTK | | | | | |
| | inhibitor of | | | | | |
| | formula (2) (5 mg/kg) | | | | | |
| | PO, QD | | | | | |
| | (d28-43) | | | | | |

(SE) = Standard error displayed in parenthesis,
AUC = Area Under the Curve
*p < 0.05 ANOVA (with Dunnett's post-hoc test) or K-W test (with Dunn's post-hoc test) vs. MTX vehicle + BTK inhibitor of formula (2) vehicle.
†p < 0.05 Student's t-test vs. MTX Vehicle + BTK inhibitor of formula (2) vehicle.
‡p < 0.05 ANOVA (with Sidak's post-hoc test) or K-W test (with Dunn's post-hoc test) vs. MTX vehicle + BTK inhibitor of formula (2) (same dose).
§p < 0.05 ANOVA (with Sidak's post-hoc test) or K-W test (with Dunn's post-hoc test) vs. MTX (same dose) + BTK inhibitor of formula (2) vehicle

Vehicle control mice had body weight loss (measured as percent change from baseline) that peaked at −16.47% on Study Day 30. Disease-induced body weight loss was significantly inhibited as compared to vehicle controls on Day 36 in mice treated with 1 mg/kg the BTK inhibitor of Formula (2), on Days 32-38 and 43 in mice treated with 5 mg/kg the BTK inhibitor of Formula (2), on Days 28-40 in mice treated with 0.3 mg/kg MTX+1 mg/kg the BTK inhibitor of Formula (2), on Days 26-40 in mice treated with 0.5 mg/kg MTX+1 mg/kg the BTK inhibitor of Formula (2), on Days 28-42 in mice treated with 0.3 mg/kg MTX+5 mg/kg the BTK inhibitor of Formula (2), and on Days 30-42 in mice treated with 0.5 mg/kg MTX+5 mg/kg the BTK inhibitor of Formula (2). Body weight loss was significantly inhibited as compared to the BTK inhibitor of Formula (2) treatment alone on Days 26-32 in mice treated with MTX (0.3 or 0.5 mg/kg)+1 mg/kg the BTK inhibitor of Formula (2) and on Days 28-30 in mice treated with 0.3 mg/kg MTX+5 mg/kg the BTK inhibitor of Formula (2). Body weight loss was significantly inhibited as compared to MTX treatment alone on Days 32-42 in mice treated with 0.3 mg/kg MTX+1 mg/kg the BTK inhibitor of Formula (2), on Days 30-34 in mice treated with 0.5 mg/kg MTX+1 mg/kg the BTK inhibitor of Formula (2), on Days 30-42 in mice treated with 0.3 mg/kg MTX+5 mg/kg the BTK inhibitor of Formula (2), and on Days 32-38 in mice treated with 0.5 mg/kg MTX+5 mg/kg the BTK inhibitor of Formula (2).

At study termination, vehicle control mice had mean absolute body weight loss of −0.94 g. Absolute body weight loss was significantly (66%) inhibited in mice treated with 5 mg/kg BTK inhibitor of Formula (2) alone as compared to vehicle controls.

Daily clinical arthritis scores differed significantly from vehicle controls over time in mice treated with 5 mg/kg the BTK inhibitor of Formula (2) or 0.5 mg/kg MTX alone and in all combination therapy groups. Clinical arthritis scores were significantly reduced on Study Days 30-43 in mice treated with 5 mg/kg the BTK inhibitor of Formula (2), on Days 25-29 in mice treated with 0.5 mg/kg MTX, on Days 28-43 in mice treated with 0.3 mg/kg MTX+the BTK inhibitor of Formula (2) (1 or 5 mg/kg), and on Days 25-43 in mice treated with 0.5 mg/kg MTX+the BTK inhibitor of Formula (2) (1 or 5 mg/kg). In mice given combination therapy, clinical arthritis scores were significantly reduced on Days 25-27 in mice treated with 0.3 mg/kg MTX+1 mg/kg the BTK inhibitor of Formula (2) as compared to treatment with 1 mg/kg the BTK inhibitor of Formula (2) alone. Clinical arthritis scores were significantly reduced on Day 28 in mice treated with 0.3 mg/kg MTX+5 mg/kg the BTK inhibitor of Formula (2) as compared to treatment with 5 mg/kg the BTK inhibitor of Formula (2) alone and on Days 30-43 as compared to treatment with 0.3 mg/kg MTX alone. Clinical arthritis scores were significantly reduced on Days 24-43 in mice treated with 0.5 mg/kg MTX+1 mg/kg the BTK inhibitor of Formula (2) as compared to treatment with 1 mg/kg the BTK inhibitor of Formula (2) alone. Clinical arthritis scores were significantly reduced on Days 25-29 in mice treated with 0.5 mg/kg MTX+5 mg/kg the BTK inhibitor of Formula (2) as compared to treatment with 5 mg/kg the BTK inhibitor of Formula (2) alone and on Days 30-43 as compared to treatment with 0.5 mg/kg MTX alone.

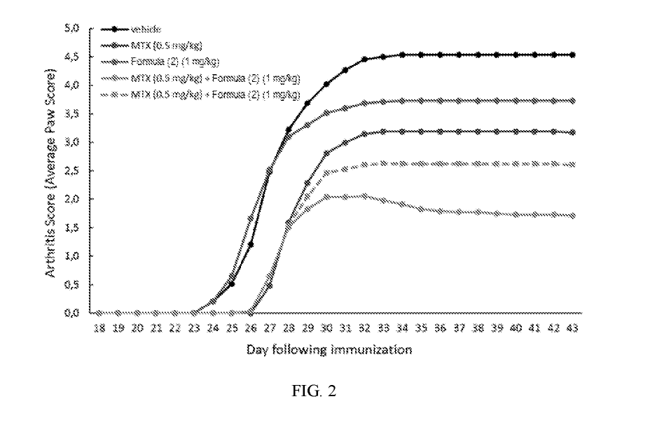

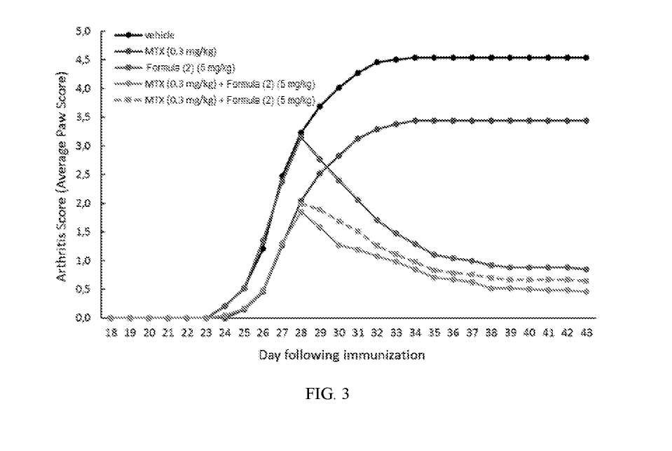

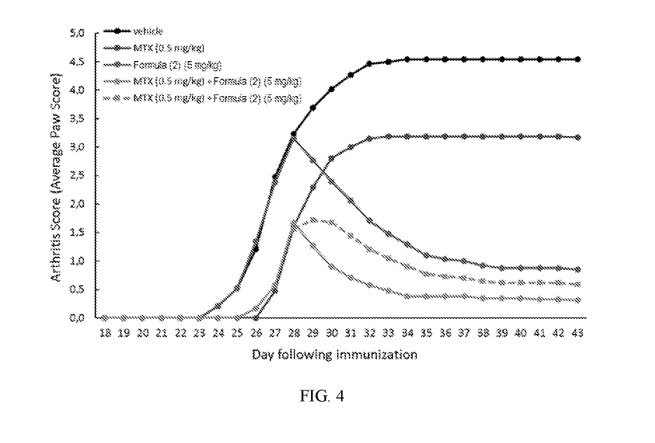

The daily arthritis score (average paw score) versus day following immunization are illustrated in FIG. 1, FIG. 2, FIG. 3, and FIG. 4.

The legends for each curve in FIG. 1 correspond to: black=vehicle; blue=MTX at 0.3 mg/kg; red=Formula (2) at 1 mg/kg; pink solid=MTX at 0.3 mg/kg and Formula (2) at 1 mg/kg; pink dashed=theoretical for MTX at 0.3 mg/kg and Formula (2) at 1 mg/kg based on data for MTX at 0.3 mg/kg and Formula (2) at 1 mg/kg.

The legends for each curve in FIG. 2 correspond to: black=vehicle; blue=MTX at 0.5 mg/kg; red=Formula (2) at 1 mg/kg; pink solid=MTX at 0.5 mg/kg and Formula (2) at 1 mg/kg; pink dashed=theoretical for MTX at 0.5 mg/kg and Formula (2) at 1 mg/kg based on data for MTX at 0.5 mg/kg and Formula (2) at 1 mg/kg.

The legends for each curve in FIG. 3 correspond to: black=vehicle; blue=MTX at 0.3 mg/kg; red=Formula (2) at 5 mg/kg; pink solid=MTX at 0.3 mg/kg and Formula (2) at 5 mg/kg; pink dashed=theoretical for MTX at 0.3 mg/kg and Formula (2) at 5 mg/kg based on data for MTX at 0.3 mg/kg and Formula (2) at 5 mg/kg.

The legends for each curve in FIG. 4 correspond to: black=vehicle; blue=MTX at 0.5 mg/kg; red-Formula (2) at 5 mg/kg; pink solid=MTX at 0.5 mg/kg and Formula (2) at 5 mg/kg; pink dashed=theoretical for MTX at 0.5 mg/kg and Formula (2) at 5 mg/kg based on data for MTX at 0.5 mg/kg and Formula (2) at 5 mg/kg.

The data of FIG. 1 to FIG. 4 illustrate that the combination of MTX and Formula (2) results in a reduction of disease incidence, evidenced by lower arthritis scores, compared to MTX and Formula (2) each alone. Furthermore, the data demonstrate a surprising synergistic effect between MTX and Formula (2). This is evidenced by the arthritis score for the combination, which was lower at each concentration combination when compared to the theoretical additive effect of the MTX and Formula (2) based on the data obtained for each active ingredient when separately administers.

Arthritis scores expressed as area under the curve (AUC) for Days 18-43 were significantly reduced as compared to vehicle controls in mice treated with 5 mg/kg the BTK inhibitor of Formula (2) (62% reduction), 0.3 mg/kg MTX+1 mg/kg the BTK inhibitor of Formula (2) (53%), 0.5 mg/kg MTX+1 mg/kg the BTK inhibitor of Formula (2) (60%), 0.3 mg/kg MTX+5 mg/kg the BTK inhibitor of Formula (2) (78%), or 0.5 mg/kg MTX+5 mg/kg the BTK inhibitor of Formula (2) (86%). Combination treatment with MTX+1 mg/kg the BTK inhibitor of Formula (2) significantly reduced AUC as compared to treatment with 1 mg/kg the BTK inhibitor of Formula (2) alone, and treatment with MTX+5 mg/kg the BTK inhibitor of Formula (2) significantly reduced AUC as compared to treatment with MTX (0.3 or 0.5 mg/kg) alone. Results of treatment on clinical scores AUC for Days 28-43 were mostly similar although reductions from treatment with 0.3 mg/kg MTX+1 mg/kg the BTK inhibitor of Formula (2) were not significant as compared to treatment with 1 mg/kg the BTK inhibitor of Formula (2) alone.

Disease incidence was reduced in mice treated with 5 mg/kg the BTK inhibitor of Formula (2) (42% incidence at termination), 0.3 mg/kg MTX+1 mg/kg the BTK inhibitor of Formula (2) (83%), 0.5 mg/kg MTX+1 mg/kg the BTK inhibitor of Formula (2) (75%), 0.3 mg/kg MTX+5 mg/kg the BTK inhibitor of Formula (2) (42%), or 0.5 mg/kg MTX+5 mg/kg the BTK inhibitor of Formula (2) (17%) as compared to vehicle controls, which had 100% incidence by Day 28.

The morphologic pathology results are as follows. Vehicle control and Isotype Control animals had histopathology changes, consistent with those seen in type II collagen-induced arthritis, in most joints, with scores ranging from minimal to severe. Microscopic alteration included infiltration of synovium and periarticular tissue with neutrophils and mononuclear inflammatory cells (inflammation), marginal zone pannus and bone resorption and cartilage damage (proteoglycan loss, chondrocyte death and collagen matrix destruction).

All six-joint mean histopathology parameters were significantly reduced in mice treated with 5 mg/kg the BTK inhibitor of Formula (2) (63-82% reductions), 0.3 mg/kg MTX+1 mg/kg the BTK inhibitor of Formula (2) (55-72%), 0.5 mg/kg MTX+1 mg/kg the BTK inhibitor of Formula (2) (66-81%), 0.3 mg/kg MTX+5 mg/kg the BTK inhibitor of Formula (2) (83-97%), or 0.5 mg/kg MTX+5 mg/kg the BTK inhibitor of Formula (2) (83-96%) as compared to vehicle controls. In mice given combination therapy, all six-joint mean histopathology parameters were significantly reduced in mice treated with 0.5 mg/kg MTX+1 mg/kg the BTK inhibitor of Formula (2) as compared to treatment with 1 mg/kg the BTK inhibitor of Formula (2) alone and in mice treated with 0.3 mg/kg MTX+5 mg/kg the BTK inhibitor of Formula (2) as compared to treatment with 0.3 mg/kg MTX alone. All six-joint mean parameters except cartilage damage scores were significantly reduced in mice treated with 0.5 mg/kg MTX+5 mg/kg the BTK inhibitor of Formula (2) as compared to treatment with 0.5 mg/kg MTX alone.

Summed six-joint mean histopathology scores were significantly reduced as compared to vehicle controls in mice treated with 5 mg/kg the BTK inhibitor of Formula (2) (73% reduction), 0.3 mg/kg MTX+1 mg/kg the BTK inhibitor of Formula (2) (64%), 0.5 mg/kg MTX+1 mg/kg the BTK inhibitor of Formula (2) (74%), 0.3 mg/kg MTX+5 mg/kg ACP-196 (91%), or 0.5 mg/kg MTX+5 mg/kg the BTK inhibitor of Formula (2) (88%). Combination treatment with 0.5 mg/kg MTX+1 mg/kg the BTK inhibitor of Formula (2) significantly reduced AUC as compared to treatment with 1 mg/kg the BTK inhibitor of Formula (2) alone, and treatment with MTX+5 mg/kg the BTK inhibitor of Formula (2) significantly reduced AUC as compared to treatment with MTX (0.3 or 0.5 mg/kg) alone.

All paw histopathology parameters were significantly reduced in mice treated with 5 mg/kg the BTK inhibitor of Formula (2) (59-82% reductions), 0.3 mg/kg MTX+1 mg/kg the BTK inhibitor of Formula (2) (53-72%), 0.5 mg/kg MTX+1 mg/kg the BTK inhibitor of Formula (2) (61-79%), 0.3 mg/kg MTX+5 mg/kg the BTK inhibitor of Formula (2) (86-97%), or 0.5 mg/kg MTX+5 mg/kg the BTK inhibitor of Formula (2) (83-97%) as compared to vehicle controls. In mice given combination therapy, all paw histopathology parameters were significantly reduced in mice treated with 0.3 mg/kg MTX+5 mg/kg the BTK inhibitor of Formula (2) as compared to treatment with 0.3 mg/kg MTX alone and in mice treated with 0.5 mg/kg MTX+5 mg/kg the BTK inhibitor of Formula (2) as compared to treatment with 0.5 mg/kg MTX alone. All paw parameters except periosteal bone formation scores were significantly reduced in mice treated with 0.5 mg/kg MTX+1 mg/kg the BTK inhibitor of Formula (2) as compared to treatment with 1 mg/kg the BTK inhibitor of Formula (2) alone.

Summed paw histopathology scores were significantly reduced as compared to vehicle controls in mice treated with 5 mg/kg the BTK inhibitor of Formula (2) (72% reduction), 0.3 mg/kg MTX+1 mg/kg the BTK inhibitor of Formula (2) (64%), 0.5 mg/kg MTX+1 mg/kg the BTK inhibitor of Formula (2) (72%), 0.3 mg/kg MTX+5 mg/kg the BTK inhibitor of Formula (2) (93%), or 0.5 mg/kg MTX+5 mg/kg the BTK inhibitor of Formula (2) (90%). Combination treatment with 0.5 mg/kg MTX+1 mg/kg the BTK inhibitor of Formula (2) significantly reduced AUC as compared to treatment with 1 mg/kg the BTK inhibitor of Formula (2) alone, and treatment with MTX+5 mg/kg the BTK inhibitor of Formula (2) significantly reduced AUC as compared to treatment with MTX (0.3 or 0.5 mg/kg) alone.

Mice treated with 5 mg/kg the BTK inhibitor of Formula (2) had significantly reduced knee inflammation (72% reduction), pannus formation (85%), and bone resorption (90%) as compared to vehicle controls. Mice treated with 0.3 mg/kg MTX+1 mg/kg the BTK inhibitor of Formula (2) had significantly reduced knee inflammation (60%) and cartilage damage (61%). Mice treated with 0.5 mg/kg MTX+1 mg/kg the BTK inhibitor of Formula (2) had significantly reduced knee inflammation (80%), pannus formation (92%), cartilage damage (83%), and bone resorption (92%). Mice treated with 0.3 mg/kg MTX+5 mg/kg ACP-196 had significantly reduced knee inflammation (77%), pannus formation (98%), cartilage damage (82%), and bone resorption (98%). Mice treated with 0.5 mg/kg MTX+5 mg/kg ACP-196 had significantly reduced knee inflammation (84%), pannus formation (85%), cartilage damage (81%), and bone resorption (88%). In mice given combination therapy, periosteal bone formation scores were significantly reduced in mice treated with 0.3 mg/kg MTX+1 mg/kg the BTK inhibitor of Formula (2) as compared to treatment with 1 mg/kg the BTK inhibitor of Formula (2) alone. All knee parameters were significantly reduced in mice treated with 0.5 mg/kg MTX+1 mg/kg the BTK inhibitor of Formula (2) as compared to treatment with 1 mg/kg the BTK inhibitor of Formula (2) alone. Mice treated with 0.3 mg/kg MTX+5 mg/kg the BTK inhibitor of Formula (2) had significantly reduced knee pannus formation, bone resorption, and periosteal bone formation as compared to treatment with 0.3 mg/kg MTX alone.

Summed knee histopathology scores were significantly reduced as compared to vehicle controls in mice treated with 5 mg/kg the BTK inhibitor of Formula (2) (74% reduction), 0.3 mg/kg MTX+1 mg/kg the BTK inhibitor of Formula (2) (64%), 0.5 mg/kg MTX+1 mg/kg the BTK inhibitor of Formula (2) (84%), 0.3 mg/kg MTX+5 mg/kg the BTK inhibitor of Formula (2) (84%), or 0.5 mg/kg MTX+5 mg/kg the BTK inhibitor of Formula (2) (84%). Combination treatment with 0.5 mg/kg MTX+1 mg/kg the BTK inhibitor of Formula (2) significantly reduced AUC as compared to treatment with 1 mg/kg the BTK inhibitor of Formula (2) alone.

Paw and six-joint mean periosteal bone widths were significantly reduced as compared to vehicle controls in mice treated 5 mg/kg the BTK inhibitor of Formula (2) (82-83% reductions), 0.3 mg/kg MTX+1 mg/kg the BTK inhibitor of Formula (2) (68-69%), 0.5 mg/kg MTX+1 mg/kg the BTK inhibitor of Formula (2) (80%), 0.3 mg/kg MTX+5 mg/kg the BTK inhibitor of Formula (2) (97%), or 0.5 mg/kg MTX+5 mg/kg the BTK inhibitor of Formula (2) (93-95%). In mice given combination therapy, knee periosteal bone widths were significantly reduced in mice treated with 0.3 mg/kg MTX+1 mg/kg the BTK inhibitor of Formula (2) as compared to treatment with 1 mg/kg the BTK inhibitor of Formula (2) alone. Paw, knee, and six-joint mean periosteal bone widths were significantly reduced in mice treated with 0.5 mg/kg MTX+1 mg/kg the BTK inhibitor of Formula (2) as compared to treatment with 1 mg/kg the BTK inhibitor of Formula (2) alone. Paw, knee, and six-joint mean periosteal bone widths were significantly reduced in mice treated with 0.3 mg/kg MTX+5 mg/kg the BTK inhibitor of Formula (2) as compared to treatment with 0.3 mg/kg MTX alone. Paw and six-joint mean periosteal bone widths were significantly reduced in mice treated with 0.5 mg/kg MTX+5 mg/kg the BTK inhibitor of Formula (2) as compared to treatment with 0.5 mg/kg MTX alone.

Following euthanasia, splenocytes were collected and analyzed for BTK occupancy. The results show that Btk occupancy with the BTK inhibitor of Formula (2) is the same in the presence or absence of MTX. This means that there is a synergistic effect between the combination of the BTK inhibitor of Formula (2) and MTX and the behavior of the drug combination is not due to MTX affecting exposure/clearance of the BTK inhibitor of Formula (2).

### Example 2—Evaluation of Synergy in Combinations of the BTK Inhibitor of Formula (2) and MTX

Combination experiments as described above were performed to determine the synergistic, additive, or antagonistic behavior of drug combinations of BTK Inhibitor of Formula (2) and MTX. Synergistic effects of combination therapy can be evaluated using the Bliss independence or fractional product method as reported in Yan et al., BMC Syst Biol., 2010, 4, 50. The Bliss independence model is defined by the equation Exy=Ex+Ey−(ExEy), where (Exy) is the additive effect of drugs x and y as predicted by their individual effects (Ex and Ey). The Bliss independence method assumes that the two inhibitors act via independent mechanisms. If the actual combined effect of the two inhibitors is equal to Exy, it is an additive effect case and there is no interaction between the two inhibitors. If the actual combined effect is lower than Exy, it is called antagonism. If the actual combined effect is higher than Exy, it is called synergism. Yan et al., BMC Syst Biol., 2010, 4, 50. Combination therapy at all doses resulted in synergistic effects on arthritis scores AUC (Days 28-43) and summed histopathology scores (all joints) as indicated by evaluation of theoretically predicted (expected) and experimentally observed inhibition of score increase.

Combination therapy at all doses resulted in synergistic effects on arthritis scores AUC (Days 28-43) and summed histopathology scores (all joints) as indicated by evaluation of expected inhibition (i.e., theoretically predicted) and experimentally observed inhibition of score increase, as shown in Table 5.

TABLE 5
Theoretical and Observed Synergistic Effects of Combination Therapy

| | | Clinical | | Histopathology | |
|---|---|---|---|---|---|
| | | Arthritis Score AUC | | Summer Scores | |
| | | Day 28-43 | | (All Joints) | |
| | | Expected | Observed | Expected | Observed |
| | | Inhibition | Inhibition | Inhibition | Inhibition |
| Group | Treatment | (%) | (%) | ( %) | (%) |
| 7 | MTX (0.3 mg/kg) | 39% | 53% | 50% | 64% |
| | PO, QD | | | | |
| | (d18-43) + BTK | | | | |
| | inhibitor of | | | | |
| | formula (2) | | | | |
| | (1 mg/kg) PO, QD | | | | |
| | (d28-43) | | | | |
| 8 | MTX (0.5 mg/kg) | 43% | 58% | 59% | 74% |
| | PO, QD | | | | |
| | (d18-43) + BTK | | | | |
| | inhibitor of | | | | |
| | Formula (2) | | | | |
| | (1 mg/kg) PO, | | | | |
| | QD (d28-43) | | | | |
| 9 | MTX (0.3 mg/kg) | 76% | 81% | 82% | 91% |
| | PO, QD | | | | |
| | (d18-43) + BTK | | | | |
| | inhibitor of | | | | |
| | formula (2) | | | | |
| | (5 mg/kg) PO, QD | | | | |
| | (d28-43) | | | | |
| 10 | MTX (0.5 mg/kg) | 78% | 88% | 85% | 88% |
| | PO, QD | | | | |
| | (d18-43) + BTK | | | | |
| | inhibitor of | | | | |
| | formula (2) | | | | |
| | (5 mg/kg) PO, QD | | | | |
| | (d28-43) | | | | |

### Example 3—a Phase 2A, 4-Week, Double-Blind, Proof-of-Concept Efficacy and Safety Study of the BTK Inhibitor of Formula (2) Versus Placebo in Subjects with Active Rheumatoid Arthritis on Background Methotrexate

The primary objective of the study will be to evaluate the efficacy of the BTK inhibitor of Formula (2) in subjects with active rheumatoid arthritis (“RA”) despite treatment with methotrexate (“MTX”). The secondary objectives will be to: (i) evaluate the safety and tolerability of Formula (2) when coadministered with methotrexate (“MTX”) in subjects with active RA; (ii) evaluate the PK and PD of Formula (2) with coadministration with MTX; and (iii) assess the effect of Formula (2) coadministration with MTX on various immune biomarkers.

The current treatment algorithm for RA includes first treatment with NSAIDS, followed by synthetic DMARDS, such as MTX, once NSAIDS become ineffective. Upon failure to respond to DMARDS and combinations thereof, patients are then treated with anti-tumor necrosis factor (anti-TNF) biologics. Ultimately when patients no longer respond to anti-TNF biologics, administration of rituximab (anti-CD20) or abatacept (CTLA4-Ig) is started. Currently, drug development of oral tyrosine kinase inhibitors for the treatment of RA is focusing on producing molecules with a better efficacy/safety profile than MTX and a similar efficacy/safety profile to biologics.

The efficacy of rituximab in treatment of patients with RA, relapsing remitting multiple sclerosis, systemic lupus erythematosus, or Sjögren's syndrome has validated B cells as an important target in autoimmune disorders. BTK is a Tec family non-receptor protein kinase, expressed in B cells, myeloid cells, osteoclasts, mast cells and platelets. The function of BTK in signaling pathways activated by the engagement of the BCR has been well established. Buggy, et al., Int. Rev. Immunol. 2012, 31, 119-132. The efficacy of BTK inhibition in other B cell driven diseases, such as B cell malignancies, is represented by the recent approval of ibrutinib (IMBRUVICA™), the first generation BTK inhibitor approved for the treatment of CLL and mantle cell lymphoma. However, the potential benefit of BTK inhibition for the treatment of autoimmune disorders is not limited to its effect on B cell activation. BTK is involved in several biologic processes, many of which affect disease progression in autoimmune disorders. BTK regulates FcγR signaling in myeloid cells and in mast cells it plays a key role in mast cell degranulation following FcεR1 activation. Jongstra-Bilen, et al., J Immunol. 2008, 181, 288-298; Ellmeier, et al., FEBS J. 2011, 278, 1990-2000. BTK regulates RANKL-induced osteoclast differentiation. Shinohara, et al., Cell 2008, 132, 794-806. BTK regulates TLR signaling and BTK inhibition is able to block B-cell activation when B-cells are stimulated via the BCR and TLR9. Kenny, et al., PLoS One, 2013, 8, e74103. BTK affects BCR-induced secretion of pro-inflammatory cytokines and chemokines by B-cells. de Rooij, et al., Blood, 2012, 119, 2590-2594; di Paolo, et al., Nat. Chem. Biol., 2011, 7, 41-50.

RA is a chronic autoimmune disease affecting 1 percent of the general population worldwide. It causes pain, stiffness, swelling, and limitation in the motion and function of multiple joints. If left inadequately treated, RA can produce destruction of one or more joints leading to deformity and permanent disability. As mentioned above, the treatment of RA has traditionally included NSAIDS, corticosteroids, and DMARDS. While these therapies provide some benefit, their efficacy has been limited. Newer, biologically based therapies include molecules that inhibit cytokine activity (TNF inhibitors, IL-1 Ra, or anti-IL-6 R mAb), block T cell-mediated co-stimulation (abatacept), or deplete B cells (rituximab). Newer generation kinase inhibitors (tofacitinib) have provided comparable benefits as the biologically based therapies. These therapies have been effective for moderate-to-severe RA and have slowed disease progression, as determined radiographically, particularly when combined with MTX. However, subjects using these therapies still routinely fail to achieve a response ≥50%. Additionally, these therapies can be associated with unfavorable side effects such as increased risk of serious infection.

A multicenter, randomized, double-blind, placebo-controlled, parallel-group clinical trial will be conducted. The on-treatment period will be four weeks with weekly visits to the clinic. There will be also a four-week safety follow-up period after the last dose of Formula (2) or placebo. Subjects meeting the eligibility criteria will be randomized in a 1:1 ratio. For four weeks, Treatment Group 1 will receive Formula (2) 15 mg once per day (QD) while on a stable dose of MTX between 7.5 and 25 mg/week. At the same time, Treatment Group 2 will receive placebo QD while on a stable dose of MTX between 7.5 and 25 mg/week. After the four weeks, enrollment will be paused while a data and safety monitoring board (DSMB) reviews unblinded pharmacokinetics and the safety results of the first 20 subjects treated. Following the review, 50 subjects will be randomized in a 1:1 ratio. Treatment Group 1 will receive Formula (2) 15 mg once per day (QD) while on a stable dose of MTX between 7.5 and 25 mg/week for four weeks. At the same time, Treatment Group 2 will receive placebo QD while on a stable dose of MTX between 7.5 and 25 mg/week for four weeks.

The inclusion criteria for patient eligibility are as follows. Subjects must meet the following criteria: (1) subjects must be able to read and understand the consent form, complete the study-related procedures, and communicate with the study staff; (2) subjects may be men or women and must be 18 to 75 years of age (inclusive); (3) subjects must have a diagnosis of RA according to the 2010 American College of Rheumatology/European League Against Rheumatism (ACR/EULAR) Classification Criteria for ≥three months before screening, and not before the age of 16 years; (4) subjects with a diagnosis of RA before 2011 must meet ACR 1987 criteria for diagnosis of RA; (5) subjects must have active RA at the time of randomization where active RA is defined as: ≥three swollen joints out of 28 joint count; ≥three tender joints out of 28 joint count; >upper limit of normal (ULN) for CRP; ≥one swollen joint must be in the proximal interphalangeal (PIP)/metacarpophalangeal (MCP)/wrist—where upper extremity with the greatest number of swollen joints in PIP/MCP/wrist will be defined as the index hand and be evaluated via MRI; (6) subjects must have started MTX treatment for ≥three months before randomization and must be on a stable MTX dose (7.5 to 25 mg/week) for ≥eight weeks before randomization and remain on a stable dose through the treatment period; (7) Sulfasalazine and hydroxychloroquine are allowed; however, subjects must be on stable dose for ≥eight weeks before randomization and remain on a stable dose through the treatment period; (8) subjects must be on oral folic or folinic acid supplementation ≥5 mg/week; (9) if using oral corticosteroids, subjects must be on a stable dose equivalent to ≤10 mg of prednisone/day for ≥4 weeks before randomization and remain on a stable dose through the treatment period; (10) if using non-steroidal anti-inflammatory drugs (NSAIDS), subjects must be on a stable dose for ≥2 weeks before randomization and remain on a stable dose through the treatment period; (11) subjects must have screening laboratory test result as follows: (i) Hemoglobin ≥8.5 g/dL (International System of Units [SI]: ≥85 g/L); (ii) White blood cells (WBC) ≥3.0×10³ cells/μL (SI: ≥3.0×10⁹ cells/L); (iii) Neutrophils ≥1.5×103 cells/μL; (SI: ≥1.5×109 cells/L); (iv) Platelets ≥100×10³ cells/μL (SI: ≥100×10⁹ cells/L); (v) Serum transaminase levels not exceeding 2.5×ULN; and (vi) Serum creatinine not exceeding 1.5 mg/dL\(SI: ≤25 μmol/L); (12) agreement to use acceptable forms of contraception during the study and for a minimum of 90 days after the last dose of MTX or 30 days after the last dose of Formula (2)/placebo, whichever is longer, if sexually active and able to bear or beget children. Examples of acceptable methods of contraception include condoms, implants, injectables, combined oral contraceptives, intrauterine devices, true sexual abstinence, or sterilized partner. Note that periodic abstinence (eg, calendar, ovulation, symptothermal, postovulation methods or withdrawal) are not acceptable methods of contraception; (13) women of child bearing potential who are sexually active with a male partner must agree to simultaneously use two forms of acceptable methods of contraception (eg, condom and with contraceptives) while on the study and for 30 days after the last dose of Formula (2)/placebo; (14) men must agree to refrain from sperm donation during the study and for 30 days after the last dose of Formula (2)/placebo; (15) are willing and able to adhere to the study visit schedule, and understand and comply with other protocol requirements.

The exclusion criteria for patient eligibility are: (1) prior malignancy, except for adequately treated basal cell or squamous cell skin cancer, in situ cervical cancer, or other cancer from which the subject has been disease free for ≥5 years; (2) evidence of tuberculosis (TB), as documented by a specific assay (purified protein derivative [PPD] or QuantiFERON®-TB Gold Test), medical history, or chest radiograph where exceptions include subjects who have documented treatment with isoniazid (INH) for ≥6 months, have completed treatment, and have no signs or symptoms of active TB; (3) body mass index (BMI) >35 kg/m2; (4) subjects unable to ambulate (eg, confined to a bed or wheelchair-bound) or are ACR functional class IV; (5) exposed to a live vaccine within 2 months of randomization; (6) life-threatening illness, medical condition or organ system dysfunction which, in the investigator's opinion, could compromise the subject's safety, interfere with the absorption or metabolism of Formula (2), or put the study outcomes at undue risk; (7) any condition that could affect Formula (2) absorption, including gastric restrictions and bariatric surgery, such as gastric bypass; (8) subjects who are pregnant, breast feeding, or planning a pregnancy (both men and women) within six months of randomization (9) subjects who have other inflammatory diseases that might confound the evaluations of benefit from Formula (2) therapy (including but not limited to systemic lupus erythematosus, inflammatory bowel disease, Felty's syndrome, Lyme disease, psoriasis, or multiple sclerosis) with secondary Sjögren's syndrome, asthma, or thyroid disease are acceptable; (10) subjects who have taken any investigational drug within the previous 30 days before randomization; (11) subjects with contraindications to whole-body MRI; (12) subjects with acute or chronic severe renal insufficiency (glomerular filtration rate [GFR]<30 mL/min/1.73 m²); (13) any prior BTK therapy; (14) prior nonresponse to a biologic agent or Janus kinase (JAK) inhibitor; (15) use of all other synthetic disease-modifying antirheumatic drugs (DMARDS) such as but not limited to leflunomide, azathioprine, cyclosporine, penicillamine or gold salts within eight weeks of randomization; (15) use of etanercept, anakinra, tofacitinib within four weeks of randomization; (16) use of abatacept, humira, infliximab, or tocilizumab within eight weeks of randomization; (17) subjects who have, or have had, a serious infection during the previous eight weeks before randomization (including, but not limited to, hepatitis, pneumonia, cellulitis, herpes zoster, or pyelonephritis), or have been hospitalized and/or received intravenous (IV) antibiotics for an infection; (18) known history of human immunodeficiency virus (HIV) or hepatitis B virus (HBV) or active infection with hepatitis C virus (HCV) (19) subjects with current signs or symptoms of clinically significant, progressive, or uncontrolled renal, hepatic, hematologic, gastrointestinal, endocrine, pulmonary, cardiac, neurologic, or psychiatric disease; (20) major surgery within 4 weeks before randomization or planned elective surgery during the study duration; (21) history of stroke, intracranial hemorrhage, or myocardial infarction within 6 months before randomization; (22) subjects requiring anticoagulation with warfarin or a vitamin K antagonist; (23) subjects who have, or have had, a substance abuse (drug, chemical, or alcohol) problem within the previous 2 years; (24) subjects who are unable to undergo multiple venipunctures because of poor tolerability or lack of easy venous access; and (25) concurrent participation in another therapeutic clinical trial.

The following efficacy parameters will be used to assess the participants. At the primary endpoint: disease activity score 28 C-reactive protein (“DAS28-CRP”) will be accessed at week four. Secondary Endpoints will be assessed as follows: (i) DAS28-CRP at weeks 1, 2, and 3; (ii) American College of Rheumatology (“ACR”) ACR20 at weeks 1, 2, 3, 4; (iii) ACR50 at weeks 1, 2, 3, 4; (iv) ACR70 at weeks 1, 2, 3, and 4; (v) individual ACR domains at weeks 1, 2, 3, and 4 such as: swollen joint count (“SJC”), tender joint count (“TJC”), health assessment questionnaire disability index (“HAQ-DI”), physician's global assessment, subject's global assessment, subject's assessment of pain, C-reactive protein (“CRP”), crythrocyte sedimentation rate (“ESR”), ACR-N at weeks 1, 2, 3, and 4, clinical disease activity index (“CDAI”) at weeks 1, 2, 3, and 4, SDAI at weeks 1, 2, 3, and 4.

Magnetic resonance imaging (MRI) of the index hand may also be evaluated using the rheumatoid arthritis MRI scoring system (“RAMRIS”) between baseline and week 4. A MRI will be performed on the most severely involved hand and wrist at baseline and week 4. Three dimensional GRE with and without gadolinium contrast and STIR images will be acquired. The MRI images will be centrally read by two experienced, independent radiologists who will be blinded to treatment assignment and the sequence of the images. Images will be scored using standard RAMRIS method.

Pharmacokinetic and pharmacodynamic parameters for Formula (2) and MTX and its metabolite will be measured in plasma and urine. Pharmacokinetic parameters will be measured in a subset of subjects (N=20) at select study centers. The plasma and urine PK of Formula (2), MTX, and 7-hydroxymethotrexate (a metabolite of MTX) will be characterized using non compartmental analysis. The following pharmacokinetic parameters will be calculated, whenever possible, from plasma and/or urine concentrations of analytes: (i) AUC0-last: Area under the plasma concentration-time curve calculated using linear trapezoidal summation from time zero to time last, where “last” is the time of the last measurable concentration; (ii) AUC0-24: Area under the plasma concentration-time curve from zero to 24 hours, calculated using linear trapezoidal summation; (iii) AUC0-inf: Area under the plasma concentration-time curve from zero to infinity, calculated using the formula: AUC0-inf=AUC0-last+Ct/λz, where λz is the apparent terminal elimination rate constant; (iv) Cmax: Maximum observed plasma concentration: (iv) Tmax: Time to maximum drug concentration (obtained without interpolation); and (v) t½: Terminal elimination half-life (whenever possible).

Pharmacodynamic parameters will be measured for the occupancy of BTK by Formula (2) in PBMCs with the aid of a biotin-tagged Formula (2) analogue probe. The effect of Formula (2) on biologic markers of B-cell function will also be evaluated, where the biomarker parameters include: (i) matrix metalloproteinase-3 (MMP3): (ii) interleukin-6 (IL-6); (iii) interleukin-8 (IL-8); (iv)C-terminal cross-linked telopeptide (CTX) and N-terminal cross-linked telopeptide (NTX); (v) rheumatoid factor (RF); (vi) osteocalcin; (vii) cartilage oligomeric matrix protein (COMP); and (viii) bone alkaline phosphatase (BAP).

1-4. (canceled)

5. A method of treating a hyperproliferative disorder, comprising co-administering, to a mammal in need thereof, therapeutically effective amounts of (1) an antifolate compound or a pharmaceutically acceptable salt thereof, and (2) a Bruton's tyrosine kinase (BTK) inhibitor or a pharmaceutically acceptable salt thereof.

6. The method of claim 5, wherein the antifolate compound is administered to the mammal before administration of the BTK inhibitor.

7. The method of claim 5, wherein the antifolate compound is administered to the mammal simultaneously with the administration of the BTK inhibitor.

8. The method of claim 5, wherein the antifolate compound is administered to the mammal after administration of the BTK inhibitor.

9. The method of claim 5, wherein the BTK inhibitor is selected from the group consisting of: 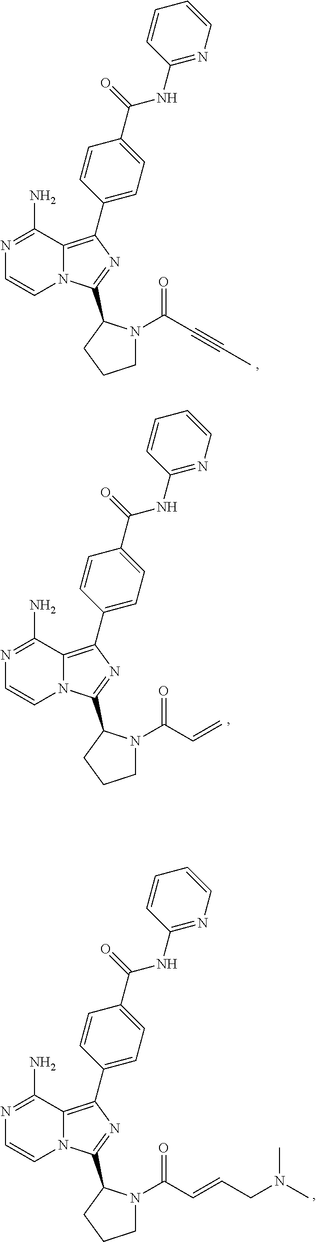 [SMILES: CC#CC(=O)N1CCC[C@H]1c1nc(-c2ccc(C(=O)Nc3ccccn3)cc2)c2c(N)nccn12 ; C=CC(=O)N1CCC[C@H]1c1nc(-c2ccc(C(=O)Nc3ccccn3)cc2)c2c(N)nccn12 ; CN(C)C/C=C/C(=O)N1CCC[C@H]1c1nc(-c2ccc(C(=O)Nc3ccccn3)cc2)c2c(N)nccn12] 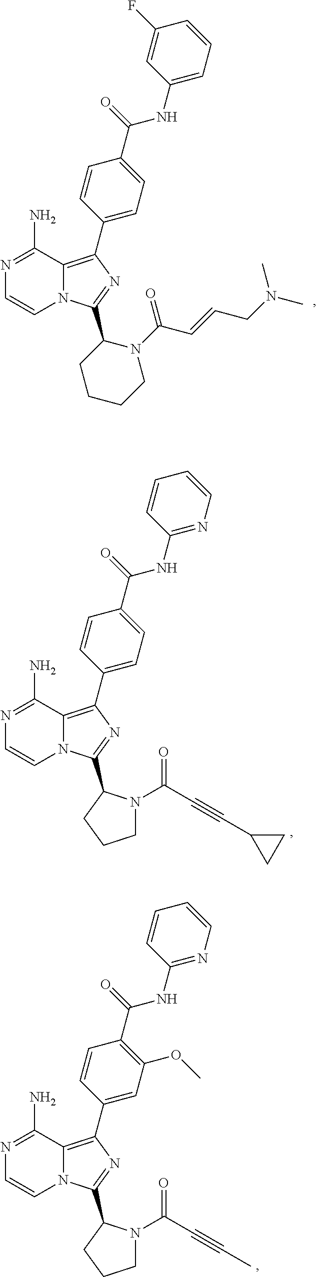 [SMILES: CN(C)C/C=C/C(=O)N1CCCC[C@H]1c1nc(-c2ccc(C(=O)Nc3cccc(F)c3)cc2)c2c(N)nccn12 ; Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12]
and pharmaceutically acceptable salts thereof.

10. The method of claim 5, wherein the BTK inhibitor is selected from the group consisting of: 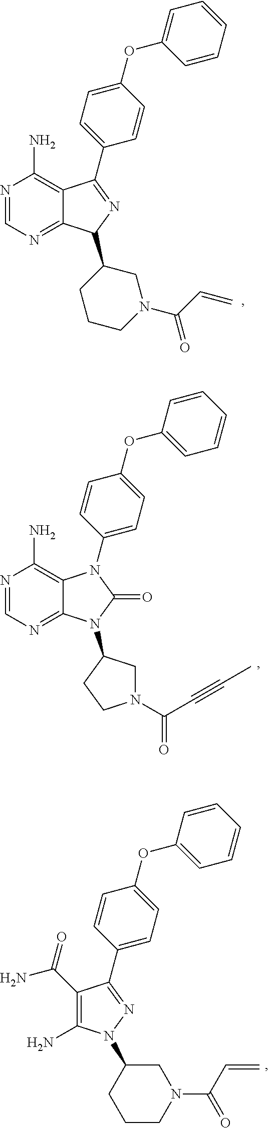 [SMILES: C=CC(=O)N1CCC[C@@H](C2N=C(c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]
and pharmaceutically acceptable salts thereof.

11. The method of claim 5, wherein the antifolate compound is selected from the group consisting of methotrexate, pemetrexed, raltitrexed, and pharmaceutically acceptable salts and combinations thereof.

12. The method of claim 5, further comprising the step of administering a therapeutically effective amount of an anti-CD20 antibody.

13. The method of claim 12, wherein the anti-CD20 antibody is selected from the group consisting of rituximab, obinutuzumab, ofatumumab, veltuzumab, tositumomab, ibritumomab, and fragments, derivatives, conjugates, variants, radioisotope-labeled complexes, biosimilars, and combinations thereof.

14. The method of claim 5, further comprising the step of administering a therapeutically effective amount of a chemotherapeutic regimen selected from the group consisting of (1) fludarabine, cyclophosphamide, and rituximab (FCR); and (2) rituximab, cyclophosphamide, doxorubicin, vincristine, and prednisone (R-CHOP).

15. The method of claim 5, further comprising the step of administering a therapeutically effective amount of a PD-1 or PD-L1 inhibitor selected from the group consisting of nivolumab, pembrolizumab, pidilizumab, durvalumab, atezolizumab, avelumab, and antigen-binding fragments, variants, conjugates, or biosimilars thereof.

16. The method of claim 5, wherein the hyperproliferative disorder is a cancer.

17. The method of claim 16, wherein the cancer is a B cell hematological malignancy.

18. The method of claim 17, wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, and myelofibrosis.

19. The method of claim 16, wherein the cancer is a solid tumor cancer.

20. The method of claim 19, wherein the solid tumor cancer is selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer.

21. The method of claim 5, wherein the hyperproliferative disorder is an inflammatory, immune, or autoimmune disorder.

22. The method of claim 21, wherein the hyperproliferative disorder is selected from the group consisting of tumor angiogenesis, chronic inflammatory disease, rheumatoid arthritis, atherosclerosis, inflammatory bowel disease, skin diseases such as psoriasis, eczema, and scleroderma, Type 1 diabetes, Type 2 diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidradenitis suppurativa, Sjögren's syndrome, psoriatic arthritis, juvenile rheumatoid arthritis, ankylosing spondylitis, Crohn's disease, lupus, lupus nephritis, human leukocyte antigen (HLA) associated diseases, autoantibodies, immunotherapy, Addison's disease, autoimmune polyendocrine syndrome type 1 (APS-1), autoimmune polyendocrine syndrome type 2 (APS-2), Grave's disease, Hashimoto's thyroiditis, polyendocrine autoimmunity, iatrogenic autoimmunity, idiopathic hypoparathyroidism, and vitiligo.

23. A method of treating a cancer in a human comprising the step of co-administering (1) a therapeutically effective amount of an antifolate compound or a pharmaceutically acceptable salt thereof, and (2) a therapeutically effective amount of a Bruton's tyrosine kinase (BTK) inhibitor or a pharmaceutically acceptable salt thereof, wherein the therapeutically effective amount is effective to inhibit signaling between a tumor cell of the cancer and at least one tumor microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts.

24. The method of claim 23, wherein the cancer is a solid tumor cancer selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer.

25. The method of claim 23, wherein the therapeutically effective amount is further effective to increase immune system recognition and rejection of the solid tumor by the human.

26-83. (canceled)


SEQUENCE LISTING (68 sequences)

SEQ ID NO: 1 - protein, 451 aa, Artificial Sequence
  rituximab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  rituximab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial Sequence
  obinutuzumab heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 219 aa, Artificial Sequence
  obinutuzumab light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 122 aa, Artificial Sequence
  ofatumumab variable heavy chain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 6 - protein, 107 aa, Artificial Sequence
  ofatumumab variable light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 7 - protein, 222 aa, Artificial Sequence
  ofatumumab Fab fragment heavy chain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 8 - protein, 211 aa, Artificial Sequence
  ofatumumab Fab fragment light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 9 - protein, 451 aa, Artificial Sequence
  veltuzumab heavy chain
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  veltuzumab light chain
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 447 aa, Artificial Sequence
  tositumomab heavy chain
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 210 aa, Artificial Sequence
  tositumomab light chain
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 13 - protein, 443 aa, Artificial Sequence
  ibritumomab heavy chain
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 14 - protein, 209 aa, Artificial Sequence
  ibritumomab light chain
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 15 - protein, 440 aa, Artificial Sequence
  nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 214 aa, Artificial Sequence
  nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 113 aa, Artificial Sequence [sequence omitted]

SEQ ID NO: 18 - protein, 107 aa, Artificial Sequence [sequence omitted]

SEQ ID NO: 19 - protein, 5 aa, Artificial Sequence
  nivolumab heavy chain CDR1
NSGMH

SEQ ID NO: 20 - protein, 17 aa, Artificial Sequence
  nivolumab heavy chain CDR2
VIWYDGSKRYYADSVKG

SEQ ID NO: 21 - protein, 4 aa, Artificial Sequence
  nivolumab heavy chain CDR3
NDDY

SEQ ID NO: 22 - protein, 11 aa, Artificial Sequence
  nivolumab light chain CDR1
RASQSVSSYLA

SEQ ID NO: 23 - protein, 7 aa, Artificial Sequence
  nivolumab light chain CDR2
DASNRAT

SEQ ID NO: 24 - protein, 9 aa, Artificial Sequence
  nivolumab light chain CDR3
QQSSNWPRT

SEQ ID NO: 25 - protein, 447 aa, Artificial Sequence
  pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 218 aa, Artificial Sequence
  pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 120 aa, Artificial Sequence [sequence omitted]

SEQ ID NO: 28 - protein, 111 aa, Artificial Sequence [sequence omitted]

SEQ ID NO: 29 - protein, 5 aa, Artificial Sequence
  pembrolizumab heavy chain CDR1
NYYMY

SEQ ID NO: 30 - protein, 16 aa, Artificial Sequence
  pembrolizumab heavy chain CDR2
GINPSNGGTNFNEKFK

SEQ ID NO: 31 - protein, 11 aa, Artificial Sequence
  pembrolizumab heavy chain CDR3
RDYRFDMGFDY

SEQ ID NO: 32 - protein, 15 aa, Artificial Sequence
  pembrolizumab light chain CDR1
RASKGVSTSGYSYLH

SEQ ID NO: 33 - protein, 7 aa, Artificial Sequence
  pembrolizumab light chain CDR2
LASYLES

SEQ ID NO: 34 - protein, 9 aa, Artificial Sequence
  pembrolizumab light chain CDR3
QHSRDLPLT

SEQ ID NO: 35 - protein, 447 aa, Artificial Sequence
  pidilizumab heavy chain
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 213 aa, Artificial Sequence
  pidilizumab light chain
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 117 aa, Artificial Sequence
  pidilizumab variable heavy chain
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 38 - protein, 106 aa, Artificial Sequence
  pidilizumab variable light chain
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 39 - protein, 451 aa, Artificial Sequence
  durvalumab (MEDI4736) heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 265 aa, Artificial Sequence
  durvalumab (MEDI4736) light chain
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 121 aa, Artificial Sequence
  durvalumab (MEDI4736)  variable heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 42 - protein, 108 aa, Artificial Sequence
  durvalumab (MEDI4736) variable light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 43 - protein, 5 aa, Artificial Sequence
  durvalumab (MEDI4736) heavy chain CDR1
RYWMS

SEQ ID NO: 44 - protein, 17 aa, Artificial Sequence
  durvalumab (MEDI4736) heavy chain CDR2
NIKQDGSEKYYVDSVKG

SEQ ID NO: 45 - protein, 12 aa, Artificial Sequence
  durvalumab (MEDI4736) heavy chain CDR3
EGGWFGELAFDY

SEQ ID NO: 46 - protein, 12 aa, Artificial Sequence
  durvalumab (MEDI4736) light chain CDR1
RASQRVSSSYLA

SEQ ID NO: 47 - protein, 7 aa, Artificial Sequence
  durvalumab (MEDI4736) light chain CDR2
DASSRAT

SEQ ID NO: 48 - protein, 9 aa, Artificial Sequence
  durvalumab (MEDI4736) light chain CDR3
QQYGSLPWT

SEQ ID NO: 49 - protein, 448 aa, Artificial sequence
  atezolizumab (MPDL3280A) heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, Artificial sequence
  atezolizumab (MPDL3280A) light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 118 aa, Artificial sequence
  atezolizumab (MPDL3280A) variable heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 52 - protein, 108 aa, Artificial sequence
  atezolizumab (MPDL3280A) variable light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 53 - protein, 10 aa, Artificial sequence
  atezolizumab (MPDL3280A) heavy chain CDR1
  variant (6)..(6): residue is D or G
GFTFSXSWIH

SEQ ID NO: 54 - protein, 18 aa, Artificial sequence
  atezolizumab (MPDL3280A) heavy chain CDR2
  variant (4)..(4): residue is S or L
  variant (10)..(10): residue is T or S
AWIXPYGGSXYYADSVKG

SEQ ID NO: 55 - protein, 9 aa, Artificial sequence
  atezolizumab (MPDL3280A) heavy chain CDR3
RHWPGGFDY

SEQ ID NO: 56 - protein, 11 aa, Artificial sequence
  atezolizumab (MPDL3280A) light chain CDR1
  variant (5)..(5): residue is D or V
  variant (6)..(6): residue is V or I
  variant (7)..(7): residue is S or N
  variant (9)..(9): residue is A or F
  variant (10)..(10): residue is V or L
RASQXXXTXXA

SEQ ID NO: 57 - protein, 7 aa, Artificial sequence
  atezolizumab (MPDL3280A) light chain CDR2
  variant (4)..(4): residue is F or T
  variant (6)..(6): residue is Y or A
SASXLXS

SEQ ID NO: 58 - protein, 9 aa, Artificial sequence
  atezolizumab (MPDL3280A) light chain CDR3
  variant (3)..(3): residue is Y, G, F or S
  variant (4)..(4): residue is L, Y, F, or W
  variant (5)..(5): residue is Y, N, A, T, G, F or I
  variant (6)..(6): residue is H, V, P, T or I
  variant (8)..(8): residue is A, W, R, P or T
QQXXXXPXT

SEQ ID NO: 59 - protein, 450 aa, Artificial Sequence
  avelumab (MSB0010718C) heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 216 aa, Artificial Sequence
  avelumab (MSB0010718C) light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 61 - protein, 120 aa, Artificial Sequence
  avelumab (MSB0010718C) variable heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 62 - protein, 110 aa, Artificial Sequence
  avelumab (MSB0010718C) variable light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 63 - protein, 5 aa, Artificial Sequence
  avelumab (MSB0010718C) heavy chain CDR1
SYIMM

SEQ ID NO: 64 - protein, 17 aa, Artificial Sequence
  avelumab (MSB0010718C) heavy chain CDR2
SIYPSGGITFYADTVKG

SEQ ID NO: 65 - protein, 11 aa, Artificial Sequence
  avelumab (MSB0010718C) heavy chain CDR3
IKLGTVTTVDY

SEQ ID NO: 66 - protein, 14 aa, Artificial Sequence
  avelumab (MSB0010718C) light chain CDR1
TGTSSDVGGYNYVS

SEQ ID NO: 67 - protein, 7 aa, Artificial Sequence
  avelumab (MSB0010718C) light chain CDR2
DVSNRPS

SEQ ID NO: 68 - protein, 10 aa, Artificial Sequence
  avelumab (MSB0010718C) light chain CDR3
SSYTSSSTRV